(19) United States
(12) Patent Application Publication
(10) Pub. No.: US 2016/0213807 A1
(43) Pub. Date: Jul. 28, 2016

(54) SOLID DRESSING FOR TREATING WOUNDED TISSUE

(71) Applicant: STB, Ltd., Bellevue, WA (US)

(72) Inventors: Martin MacPhee, Darnestown, MD (US); Dawson Beall, Germantown, MD (US)

(21) Appl. No.: 15/089,498

(22) Filed: Apr. 2, 2016

Related U.S. Application Data
(63) Continuation of application No. 14/599,516, filed on Jan. 18, 2015.
Application No. 14/599,516 is a continuation of application No. 13/843,604, filed on Mar. 15, 2013, now abandoned.
Application No. 13/843,604 is a continuation-in-part of application No. 11/882,872, filed on Aug. 6, 2007, now Pat. No. 8,445,009.
(60) Provisional application No. 60/835,423, filed on Aug. 4, 2006.

(51) Int. Cl. A61L 15/44; A61L 15/38; A61L 15/32; A61L 15/64

(52) U.S. Cl. CPC A61L 15/44; A61L 15/64; A61L 15/38; A61L 15/32; A61L 2300/418; A61L 2400/04

(57) ABSTRACT

Disclosed are solid dressings for treated wounded tissue in mammalian patients, such as a human, comprising a haemostatic layer consisting essentially of a fibrinogen component and a fibrinogen activator, wherein the haemostatic layer(s) is cast or formed from a single aqueous solution containing the fibrinogen component and the fibrinogen activator. Also disclosed are methods for treating wounded tissue using these dressings and frozen compositions useful for preparing the haemostatic layer(s) of these dressings.

### CROSS REFERENCE TO RELATED APPLICATIONS

The present application is a continuation-in-part of application Ser. No. 11/882,872, filed Aug. 6, 2007, which is incorporated herein by reference in its entirety.

### FIELD OF THE INVENTION

The present invention relates to a process for producing a solid dressing for treating wounded tissue in a mammalian patient, such as a human, and to the dressings and intermediates produced thereby.

### BACKGROUND OF THE INVENTION

The materials and methods available to stop bleeding in pre-hospital care (gauze dressings, direct pressure, and tourniquets) have, unfortunately, not changed significantly in the past 2000 years. See L. Zimmerman et al., Great Ideas in the History of Surgery (San Francisco, Calif.: Norman Publishing; 1993), 31. Even in trained hands they are not uniformly effective, and the occurrence of excessive bleeding or fatal hemorrhage from an accessible site is not uncommon. See J. M. Rocko et al., J. Trauma 22:635 (1982).

Mortality data from Vietnam indicates that 10% of combat deaths were due to uncontrolled extremity hemorrhage. See SAS/STAT Users Guide, 4th ed. (Cary, N.C.: SAS Institute Inc; 1990). Up to one third of the deaths from ex-sanguination during the Vietnam War could have been prevented by the use of effective field hemorrhage control methods. See SAS/STAT Users Guide, 4th ed. (Cary, N.C.: SAS Institute Inc.; 1990).

Although civilian trauma mortality statistics do not provide exact numbers for pre-hospital deaths from extremity hemorrhage, case and anecdotal reports indicate similar occurrences. See J. M. Rocko et al. These data suggest that a substantial increase in survival can be affected by the pre-hospital use of a simple and effective method of hemorrhage control.

Among the alternatives to gauze bandages, include fibrin based bandages. The idea behind the bandage was to create a wound material that would not have to be removed from the wound site once bleeding was controlled. For example, U.S. Pat. No. 2,492,458 and U.S. Pat. No. 4,442,655.

Conventionally, gauze and these fibrin based products used to control bleeding have depended upon the patient's body to form a blood clot that halts the bleeding. The conventional outline of this overall process is shown in FIG. 1. Key components in the latter stages of clot formation include fibrinogen, thombin and Factor XIII which interact to transform fibrinogen into insoluble, cross-linked fibrin that is covalently bonded to injured tissue, sealing it and achieving hemostasis. This process is outlined in FIG. 6. During this process, the soluble monomer fibrinogen (itself consisting of three protein chains, Aα, Bβ and γ) is first acted upon by thrombin, a proteinase that cleaves off a small portion of each of the Aα and Bβ chains, releasing the fibrinopeptides A and B. This allows the monomers to associate into dimers and increasingly larger multimers via interactions involving the newly exposed regions of the α and β chains. As these polymers grow in length, they are referred to as protofibrils, and then fibrils and fibers. Their solubility under physiological conditions decreases as the length of the polymers increase. If the reaction is allowed to continue long enough, eventually these fibers may become insoluble on their own.

However there is another polymerization reaction also occurring when Factor XIII is present. Factor XIII is also subject to cleavage by thrombin, resulting in Factor XIIIa Factor XIIIa is a transaminase that covalently cross-links the γ chains of fibrinogen. In native fibrinogen these are not spatially available for crosslinking by Factor XIIIa, however following the action of thrombin upon fibrinogen, and the self-assembly of fibrinogen into polymers of various lengths, the necessary amino acids are aligned in sufficient proximity for Factor XIIIa to cross link the fibrinogen polymers into an insoluble three dimensional network. If proteins from injured tissue are very close to the outer edge of the growing fibrin mass they can also be covalently crosslinked to the fibrin by Factor XIIIa. If this occurs fast enough, and the fibrin mass is dense enough and strong enough, the injured tissue may be sealed off, halting fluid loss. If the injured tissue was bleeding, than this sealing can result in hemostasis.

Once too many of the cross linkable residues have been internally crosslinked the surface of the fibrin mass can no longer be effectively attached to injured tissue. At this point is ceases to be an active hemostatic material, and is simply a fairly biocompatible material to press onto a wound. Interestingly, this attribute was harnessed by Behring, Ingraham, and Bailey et al. in the 1940's when they developed products from various forms of fully polymerized, non-crosslinkable fibrin which was no longer cross-linkable to tissue in situ, which therefore saw use as a hemostatic material for treating very mild oozing and for providing pressure to tamponade injured tissue while awaiting the patient's own clotting system to stop the bleeding by serving as materials to be stuffed into injured tissue cavities and similar uses. They manufactured these by mixing fibrinogen and thrombin under conditions and for sufficient times to cause them to be fully polymerized. While there resulting compositions were useful, they were not highly hemostatic and instead served primarily as space fillers.

A similar approach was taken many years later by Stroetmann, U.S. Pat. No. 4,442,655 who reacted fibrinogen and thrombin to form a mixture that could then be frozen and lyophylized. According to Stroetmann, the resulting material may consist of between 10 to 95% of “fibrin”. However the term “fibrin” is not clearly defined in this Patent, and as shown in FIG. 6 this means it is unclear which of the many stages of conversion of fibrinogen to fibrin is being referred to in Stroetmann. This uncertainty may lead to confusion regarding the compositions described in this Invention and those described by Stroetmann. It is important therefore to clearly understand the distinction between the use of the term fibrin in this application and that implied in Stroetmann.

Since Stroetmann provides no definition of “fibrin” the most that can be done is to examine the methods used to measure “fibrin” in Stroetmann, and compare the resulting material to that defined in this application. Stroetmann provides only an incomplete description of how they measure the amount of “fibrin” in their compositions, however it is clear that the material they are referring to as “fibrin” is soluble in physiological conditions of 0.9% NaCl as this is the solvent used to extract the “fibrin” in their composition for analysis (See Stroetmann, Example 1, Col 14, L30-39).

In contrast our definition of fibrin requires that the material is insoluble under physiological conditions. Thus to extract the fibrin from the compositions described herein physiological conditions are ineffective, and instead 5M Urea is used. It should be noted that this is the current, conventional use of the term “fibrin,” when not modified in some way.

Due to the vagueness of the Stroetmann Patent the best that can be inferred is that they were measuring some soluble protein that they distinguish from fibrinogen. This precludes the possibility that they were referring to fibrin as it is defined today, and indeed their definition is inconsistent, or at least incomplete, with the definition in use at the time their Patent was written. In summary, while it is not possible to know exactly what is meant by the term “fibrin” in Stroetmann, it is certain that it is not the conventional meaning, nor the modern meaning, and finally, not the meaning as defined in this application.

There are now in use a number of newer haemostatic agents that have been developed to overcome the deficiencies of traditional gauze bandages and fibrin fleece bandages. These haemostatic agents include the following:
Microporous polysaccharide particles (TraumaDEX®, Medafor Inc., Minneapolis, Minn.);
Zeolite (QuikClot®, Z-Medica Corp, Wallington, Conn.);
Acetylated poly-N-acetyl glucosamine (Rapid Deployment Hemostat™ (RDH), Marine Polymer Technologies, Danvers, Mass.);
Chitosan (HemCon® bandage, HemCon Medical Technologies inc., Portland Oreg.);
Liquid Fibrin Sealants (Tisseel V H, Baxter, Deerfield, Ill.)
Human fibrinogen and thrombin on equine collagen (TachoComb-S, Hafslund Nycomed Pharma, Linz, Austria);
Microdispersed oxidized cellulose (m•Doc™, Alltracel Group, Dublin, Ireland);
Propyl gallate (Hemostatin™, Analytical Control Systems Inc., Fishers, Ind.);
Epsilon aminocaproic acid and thrombin (Hemarrest™ patch, Clarion Pharmaceuticals, Inc);
Purified bovine corium collagen (Avitene® sheets (non-woven web or Avitene Microfibrillar Collagen Hemostat (MCH), Davol, Inc., Cranston, R.I.);
Controlled oxidation of regenerated cellulose (Surgicel®, Ethicon Inc., Somerville, N.J.);
Aluminum sulfate with an ethyl cellulose coating (Sorbastace Microcaps, Hemostace, LLC, New Orleans, La.);
Microporous hydrogel-forming polyacrylamide (BioHemostat, Hemodyne, Inc., Richmond Va.); and
Recombinant activated factor VII (NovoSeven®, NovoNordisk Inc., Princeton, N.J.).
   These agents have met with varying degrees of success when used in animal models of traumatic injuries and/or in the field.

One such agent is a starch-based haemostatic agent sold under the trade name TraumaDEX™. This product comprises microporous polysaccharide particles that are poured directly into or onto a wound. The particles appear to exert their haemostatic effect by absorbing water from the blood and plasma in the wound, resulting in the accumulation and concentration of clotting factors and platelets. In two studies of a lethal groin wound model, however, this agent showed no meaningful benefit over standard gauze dressings. See McManus et al., Business Briefing: Emergency Medical Review 2005, pp. 76-79 (presently available on-line at www.touchbriefings.com/pdf/1334/Wedmore.pdf).

Another particle-based agent is QuickClot™ powder, a zeolite granular haemostatic agent that is poured directly into or onto a wound. The zeolite particles also appear to exert their haemostatic effect through fluid absorption, which cause the accumulation and concentration of clotting factors and platelets. Although this agent has been used successfully in some animal studies, there remains concern about the exothermic process of fluid absorption by the particles. Some studies have shown this reaction to produce temperatures in excess of 143° C. in vitro and in excess of 50° C. in vivo, which is severe enough to cause third-degree burns. See McManus et al., Business Briefing: Emergency Medical Review 2005, at 77. The exothermic reaction of QuikClot™ has also been observed to result in gross and histological tissue changes of unknown clinical significance. Acheson et al., J. Trauma 59:865-874 (2005).

Unlike these particle-based agents, the Rapid Deployment Hemostat™ appears to exert its haemostatic effect through red blood cell aggregation, platelet activation, clotting cascade activation and local vasoconstriction. The Rapid Deployment Hemostat™ is an algae-derived dressing composed of poly-N-acetyl-glucosamine. While the original dressing design was effective in reducing minor bleeding, it was necessary to add gauze backing in order to reduce blood loss in swine models of aortic and liver injury. See McManus et al., Business Briefing: Emergency Medical Review 2005, at 78.

Another poly-N-acetyl-glucosamine-derived dressing is the HemCon™ Chitosan Bandage, which is a freeze-dried chitosan dressing purportedly designed to optimize the mucoadhesive surface density and structural integrity of the chitosan at the site of the wound. The HemCon™ Chitosan Bandage apparently exerts its haemostatic effects primarily through adhesion to the wound, although there is evidence suggesting it may also enhance platelet function and incorporate red blood cells into the clot it forms on the wound. This bandage has shown improved hemostasis and reduced blood loss in several animal models of arterial hemorrhage, but a marked variability was observed between bandages, including the failure of some due to inadequate adherence to the wound. See McManus et al., Business Briefing: Emergency Medical Review 2005, at 79.

Liquid fibrin sealants, such as Tisseel V H, have been used for years as an operating room adjunct for hemorrhage control. See J. L. Garza et al., J. Trauma 30:512-513 (1990); H. B. Kram et al., J. Trauma 30:97-101(1990); M. G. Ochsner et al., J. Trauma 30:884-887 (1990); T. L. Matthew et al., Ann. Thorac. Surg. 50:40-44 (1990); H. Jakob et al., J. Vasc. Surg., 1:171-180 (1984). The first mention of tissue glue used for hemostasis dates back to 1909. See Current Trends in Surgical Tissue Adhesives: Proceedings of the First International Symposium on Surgical Adhesives, M. J. MacPhee et al., eds. (Lancaster, Pa.: Technomic Publishing Co; 1995). Liquid fibrin sealants are typically composed of fibrinogen and thrombin, but may also contain Factor XIII/XIIIa, either as a by-product of fibrinogen purification or as an added ingredient (in certain applications, it is therefore not necessary that Factor XIII/Factor XIIIa be present in the fibrin sealant because there is sufficient Factor XIII/XIIIa, or other transaminase, endogenously present to induce fibrin formation). As liquids, however, these fibrin sealants have not proved useful for treating traumatic injuries in the field.

Dry fibrinogen-thrombin dressings having a collagen support (e.g. TachoComb™, TachoComb™ H and TachoSil available from Hafslund Nycomed Pharma, Linz, Austria) are also available for operating room use in many European countries, and a similar product is now available in the U.S. See U. Schiele et al., Clin. Materials 9:169-177 (1992). While these fibrinogen-thrombin dressings do not require the pre-mixing needed by liquid fibrin sealants, their utility, particularly for field applications, is limited by a requirement for storage at 4° C. and the necessity for pre-wetting with saline solution prior to application to the wound. These dressings are also not effective against high pressure, high volume bleeding. See Sondeen et al., J. Trauma 54:280-285 (2003).

Dry fibrinogen/thrombin dressings for treating wounded tissue that do not require specialized handling or storage at 4° C. were developed by from the American Red Cross (ARC). The first, as disclosed in U.S. Pat. Nos. 7,189,410 and 7,196,054 consist of a backing material and a mixture of powdered fibrinogen and thrombin. These compositions, while useful, suffer from difficulties attributable to the powdered physical state of their active ingredients, fibrinogen and thrombin. When powders composed of different materials are mixed there always remain distances between the particles of different material that are large compared to the size of the molecules of which the particles are formulated. In order to make a single, seamless, homogenous layer of highly hemostatic fibrin, this requires that the molecules be rapidly released from the particles by dissolving in a liquid (solvent), then the liquids must mix perfectly in situ and only then react, and that this process take place evenly on both a micro and macro scale through the entire space separating all the particles. This is inherently impossible for several reasons and the result is that the fibrin formed in these dressings is inherently non-homogenous, with stronger and weaker regions with correspondingly more or fewer attachment sites for injured tissue.

Among the reasons this cannot be achieved when mixing powdered forms of fibrinogen and thrombin is that the particles consist primarily of proteins. When attempting to hydrate these particles, they form hydrogel layers on the surfaces of the individual particles which slow the dissolution and/or diffusion of additional protein molecules from the dry particle in the center, thru the hydrogel outer layer, and into the medium between the particles.

This is more of an issue with fibrinogen, due to its high molecular weight and its greater inherent tendency to form hydrogels. Thrombin formulations may experience this problem, although to a lesser degree. As a result, upon initial hydration, the concentration of fibrinogen in solution that is available to interact with thrombin, other fibrinogen cleavage forms and polymers, and Factor XIIIa, will be quite low, with the bulk of the fibrinogen tied up within the particles and hydrogel layer around the particle. The concentration within the liquid that is available to react to form fibrin will be quite low, rising only when more fibrinogen dissolves from the outer layer of the particle, travels thru the hydrogel layer without being trapped, and is released into the surrounding medium. This process may take place with varying rates from different particles and in different locations in or on the surface of the dressing, and will be more variable when the particle size and geometry are more varied, and when the application of the solvent to the mixture is also variable with respect to volume, pressure and composition. Finally, the physical act of application of such a dressing to a wound will impart forces that serve to randomly transport materials in solution. The result of all these is both a random and a varying gradient transport of the reactants within the resulting solution.

This produces variability in the concentration of fibrinogen in various parts of the area treated by the dressing. Since fibrinogen concentration is a determinate of the strength of adherence and the resulting hemostatic efficacy, any product that experiences a varying concentration of fibrinogen in solution across the area it is applied to will have a decreased reliability and a decreased maximum hemostatic efficacy since a failure in one area that allows bleeding is seen as a product failure.

The inherent hemostatic and adherence performance variability of non-homogenous fibrin sealant dressings is sensitive not only to the fibrinogen concentration in the fibrinogen/thrombin solution that results from hydration of the dressing, but also to the ratio of Thrombin to Fibrinogen in solution (the “T:F ratio”). Varying T:F ratios produce different fibrin structures from solutions containing the same concentrations of fibrinogen with different characteristics. So again, dressings formed from powders made of individual particles of fibrinogen and separate particles of thrombin will, upon application to a wound (or the application of a solvent to the dressing) have inherently varying levels of fibrinogen, and varying T:F ratios, both of which will contribute to variability in the structure and efficacy of the dressing over the area it is applied to.

As mentioned previously, this results in the fibrin forming as a non-homogenous mass, with stronger and weaker regions with differing internal tensile strengths, elastic and adhesive properties, combined with significantly variable concentrations of the number of potential sites for attachment of the fibrin to injured tissue per unit of area over the area being treated. The resulting mass is therefore less hemostatically potent, as any hemostatic or sealing wound treatment is only as effective as its weakest point where blood or other fluids may leak out. For these reasons, particle-based fibrinogen/thrombin dressings are inherently limited in their efficacy. And structured fibrinogen/thrombin dressings may also experience performance limits associated with poor mixing and inconsistent/gradiant fibrinogen concentrations and/or T:F ratios (J Biomed Mater Res B Appl Biomater. 2004 Jul. 15; 70(1):114-21. Structural design of the dry fibrin sealant dressing and its impact on the hemostatic efficacy of the product. Pusateri A E, Kheirabadi B S, Delgado A V, Doyle J W, Kanellos J, Uscilowicz J M, Martinez R S, Holcomb J B, Modrow H E.).

Additional limits imposed by the powdered product format relate to the mass of powdered material that can be delivered to the wound site, the requirements for one or more methods to maintain the powdered material adhering to the backing, and the ability to deliver the material to wounds that are non-planar due to difficulties in delivering the resulting fibrinogen/thrombin material to all faces of the wound without interference from the backing.

To avoid premature reaction, previous attempts to manufacture fibrinogen/thrombin solid dressings have emphasized the separation of the fibrinogen and thrombin components as much as possible in order to prevent them from forming too much fibrin prior to use of the dressing. For example, the fibrinogen-thrombin dressings having a collagen support (e.g. TachoComb™, TachoComb™ H and TachoSil) available from Hafslund Nycomed Pharma are prepared by suspending particles of fibrinogen and thrombin in a non-aqueous liquid and then spraying the suspension onto the collagen base. The use of a non-aqueous environment, as opposed to an aqueous one, is intended to prevent excessive interaction between the fibrinogen and thrombin.

There remains therefore a need for a fibrinogen/thrombin dressing in a format that does not suffer from the inherent limitations and problems of powder-based dressings. Many of the barriers to creating such a product would be solved if it were possible to create a mixture of the active ingredients at the molecular level, without them reacting during manufacturing and storage, while retaining their ability to react completely and promptly upon application to wounded tissue. Both Larson and Stroetmann failed to address these needs, as they both stressed the requirement that the active ingredients must react during manufacture in order to form “fibrin” that was subsequently preserved in its post-reaction form for use primarily as a space filling material. Stroetmann further avows that their material may serve as a “wound toilet material”, a term without contemporaneous or contemporary meaning in the U.S., but which may refer to the British term for cleaning a wound, not for hemostasis, sealing or space filling.

As the final object is a dressing that delivers fibrinogen and thrombin to a wound where it may rapidly hydrate the problem may at first seem simple, however it is greatly complicated by two main factors. The first is that the reactants, fibrinogen, thrombin and possibly Factor XIII, are all highly reactive when in solution. This precludes the development of some form of single liquid formation applied to a backing, and in practice it severely limits the ability to manufacture dressings with fibrinogen/thrombin (+Factor XIII) premixed at a molecular level.

One attempt to deal with this limitation was to disperse fibrinogen and thrombin into non-aqueous liquids, and then spray the resulting suspensions onto a backing as separate layers. The resulting product required a prolonged hydration time, and was poorly adherent and only weakly hemostatic, due to the effects of the non-aqueous liquids upon the proteins, which tended to denature and to form a sticky, slow to hydrate mass, along with the problems with mass transport and mixing of the layered components.

Another approach is disclosed in U.S. Pat. No. 6,762,336, this particular dressing is composed of a backing material and a plurality of layers, the outer two of which contain fibrinogen (but no thrombin) while the inner layer contains thrombin and calcium chloride (but no fibrinogen). In this case, the proteins were not suspended in non-aqueous liquids, but rather were kept in separate liquid solutions. In order to prevent reaction to form fibrin during manufacture, each layer had to be deposited individually, and the entire product frozen, producing a layered structure. However, this still left the ingredients unmixed, and upon hydration the same problems of arising from inadequate uniform mixing arose, leading to a variable structure in fibrin produced when used to treat wounds, and attendant limitations on hemostatic efficacy. Still, this dressing was an improvement that showed success in several animal models of hemorrhage, but there were also physical drawbacks that resulted from the layered structure. The bandage was fragile, inflexible, and had a tendency to break apart when handled. See McManus et al., Business Briefing: Emergency Medical Review 2005, at 78.; Kheirabadi et al., J. Trauma 59:25-35 (2005).

Other fibrinogen/thrombin-based dressings have also been proposed that seek to prevent the reaction of fibrinogen and thrombin. For example, use of non-aqueous mixtures or matrices: U.S. Pat. No. 4,683,142 discloses a resorptive sheet material for closing and healing wounds which consists of a glycoprotein matrix, such as collagen, containing coagulation proteins, such as fibrinogen and thrombin, and U.S. Pat. No. 6,056,970 discloses dressings composed of a bioabsorbable polymer, such as hyaluronic acid or carboxymethylcellulose, and a haemostatic composition composed of powdered thrombin and/or powdered fibrinogen. Use of separate layers: U.S. Pat. No. 5,702,715 discloses a reinforced biological sealant composed of separate layers of fibrinogen and thrombin, at least one of which also contains a reinforcement filler such as PEG, PVP, BSA, mannitol, FICOLL, dextran, myo-inositol or sodium chlorate, and U.S. Patent Application Publication No. US 2006/0155234 A1 discloses a dressing composed of a backing material and a plurality of fibrinogen layers which have discrete areas of thrombin between them. And, use of particles adhered to a bandage as in U.S. Pat. No. 7,189,410 discloses a bandage composed of a backing material having thereon: (i) particles of fibrinogen; (ii) particles of thrombin; and (iii) calcium chloride.

Furthermore, the dressing must be homogenous, as all areas of the dressing must function equally well in order to assure its successful use. The dressing must also hydrate rapidly and without significant or special efforts. Relatively flat dressings are generally preferred, with curling or irregular, non-planar structures to be avoided if possible (these ten to interfere with effective application and, in some instances, may result in poor performance). Flexibility is another characteristic that is greatly preferred, both to improve performance and to increase the number of wound geometries and locations that can be treated effectively. Although known fibrinogen/thrombin solid dressings may be flexible when hydrated, they do not possess sufficient moisture content prior to hydration to be flexible. See, e.g., Sondeen et al., J. Trauma 54:280-285 (2003)); Holcomb et al,: J. Trauma, 55 518-526; McManus & Wedmore, Emergency Medicine Review, pp76-79, 2005.

The amount of fibrin present in the dressing prior to application must be kept as low as possible for several reasons. First, the presence of insoluble fibrin during manufacture normally results in poor quality dressings, which can exhibit decreased integrity, lack of homogeneity and difficult/slow hydration. These consequences can usually be detected visually by one of skill in the art. They may also show a decreased capability to adhere to injured tissue and stop bleeding, presumably due to the necessary cross linking sites on the γ chains that are required to cross link to exposed sites on injured tissue being instead cross linked internally to other sites within or on the dressing, rendering them unavailable for attachment to the injured tissue.

For example, the presence of pre-formed fibrin in a fibrinogen/thrombin-based solid dressing can be detected visually by deviations from a homogenous surface appearance. In particular, a rough or lumpy appearance frequently signals that there are significant masses of fibrin that have formed during manufacture and will likely impede future performance. Solid, smooth & glossy “sheets” on the surface of solid dressings are also signs of fibrin that will tend to slow (or even block) hydration during use. Excessive curling-up of a solid dressing is another sign that a significant amount of fibrin has formed during manufacture. Upon addition of water or an aqueous solution, dressings with excessive fibrin content are slow to hydrate and often require forceful application of the liquid, sometimes with mechanical penetration of the surface, in order to initiate hydration. Moreover, once hydrated, dressings with a significant amount of pre-formed fibrin usually have a mottled and distinctly non-homogenous appearance.

The amount of pre-formed fibrin can also be assessed by various biochemical assays, such as the method described in U.S. Patent Application Publication No. US 2006/0155234 A1. According to this assay, the conversion of the fibrinogen γ chains to cross-linked γ-γ dimers is used as an indication of the presence of fibrin (the proportion of γ chain that is converted to γ-γ dimer being a measure of the amount of fibrin produced).

Other assays could assess changes in the other component chains of fibrinogen, such as the conversion of the Aα chain into free α chain and fibrinopeptide A or the conversion of the Bβ chain into free β chain and fibrinopeptide B. These changes can be monitored by gel electrophoresis in a similar manner to the γ to γ-γ conversion described in U.S. Patent Application Publication No. US 2006/0155234 A1. Interestingly, in U.S. Patent Application Publication No. US 2006/0155234 A1, up to 10% γ-γ dimerization was reported This observation may account for the delamination and/or cracking observed in some of these dressings.

Accordingly, there remains a need in the art for a solid dressing that can be used to treat wounded tissue, particularly wounded tissue resulting from traumatic injury in the field.

### SUMMARY OF THE INVENTION

It is therefore an object of the present invention to provide a solid dressing that can treat wounded mammalian tissue, particularly wounded tissue resulting from a traumatic injury. It is further an object of the present invention to provide a method of treating wounded mammalian tissue, particularly human tissue. Other objects, features and advantages of the present invention will be set forth in the detailed description of preferred embodiments that follows, and will in part be apparent from that description and/or may be learned by practice of the present invention. These objects and advantages will be realized and attained by the compositions and methods described in this specification and particularly pointed out in the claims that follow.

In accordance with these and other objects, a first embodiment of the present invention is directed to a solid dressing comprising a haemostatic layer wherein said haemostatic layer comprises dried fibrinogen and thrombin, said dried fibrinogen and thrombin being dried from a single aqueous mixture of fibrinogen and thrombin.

Another embodiment is a haemostatic dressing comprising a haemostatic layer consisting essentially of a substantially unreacted solid mixture of fibrinogen and thrombin molecules dried from a single aqueous medium.

Another embodiment is a solid dressing comprising a haemostatic layer comprising substantially unreacted fibrinogen and thrombin, wherein said fibrinogen and said thrombin are dried from a single aqueous solution forming said solid haemostatic layer; and wherein said fibrinogen and thrombin are substantially unreacted until said solid dressing comes into contact with an aqueous fluid.

Another embodiment is a solid dressing comprising a haemostatic layer comprising a substantially unreacted mixture of a fibrinogen component and a fibrinogen activator molecules; wherein said fibrinogen component and fibrinogen activator molecules are dried together from a single aqueous medium forming said solid haemostatic layer.

Another embodiment is directed to a method of treating wounded tissue using a solid dressing comprising at least one haemostatic layer consisting essentially of a fibrinogen component and a fibrinogen activator, wherein the haemostatic layer(s) is cast or formed from a single aqueous solution containing the fibrinogen component and the fibrinogen activator.

Another embodiment is directed to a method of treating wounded tissue using a solid dressing comprising at least one haemostatic layer consisting essentially of a fibrinogen component and a fibrinogen activator, wherein the haemostatic layer(s) is cast or formed as a single piece.

Another embodiment is directed to a composition consisting essentially of a mixture of a fibrinogen component, a fibrinogen activator and water, wherein the composition is frozen and is stable at reduced temperature for at least 24 hours.

Another embodiment is directed to a process for producing a solid dressing comprising a haemostatic layer comprising a substantially unreacted mixture of fibrinogen and thrombin molecules; wherein said process comprises creating an aqueous mixture of fibrinogen and thrombin at a temperature sufficiently low to permit it to be introduced into a mold which is then cooled sufficiently to freeze the mixture within the mold molecules until the mixture freezes without substantial reaction(s) occurring between the fibrinogen and thrombin.

Another embodiment is directed to a process for producing a solid dressing comprising a haemostatic layer comprising a substantially unreacted mixture of fibrinogen and thrombin molecules; wherein said process comprises creating an aqueous mixture of fibrinogen and thrombin at a temperature sufficiently low to permit it to be introduced into a mold which is then cooled sufficiently to freeze the mixture within the mold molecules until the mixture freezes without substantial fibrin formation within the mixture.

It is to be understood that the foregoing general description and the following detailed description of preferred embodiments are exemplary and explanatory only and are intended to provide further explanation, but not limitation, of the invention as claimed herein.

### BRIEF DESCRIPTION OF DRAWINGS

FIG. 1 is an overview of the human clotting cascade as provided by ERL's website (www.enzymeresearch.co.uk/coag.htm).

FIG. 2 is a diagram of the set-up for the ex vivo porcine arteriotomoy assay described herein.

FIGS. 3A-3C are graphs showing the results achieved in Example 1.

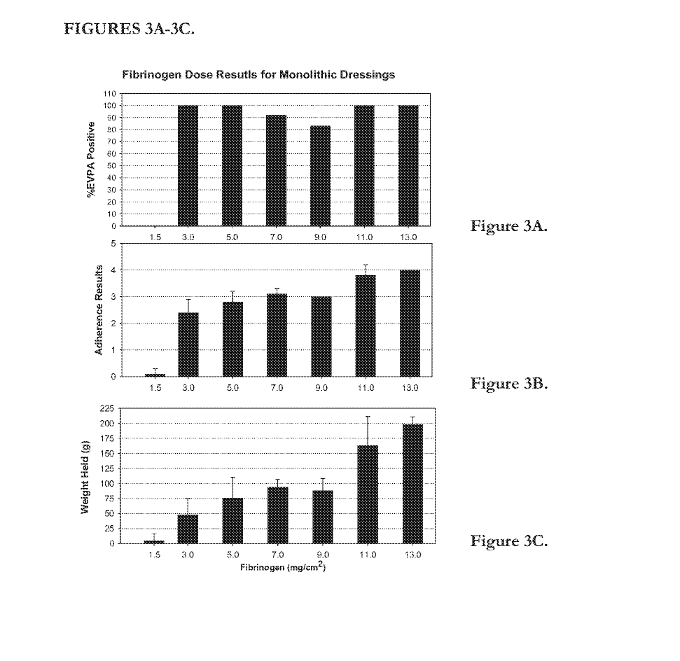

FIG. 4A and FIG. 4B are graphs depicting the results of the EVPA and Adherence Assays for the dressings made in Examples 6-12.

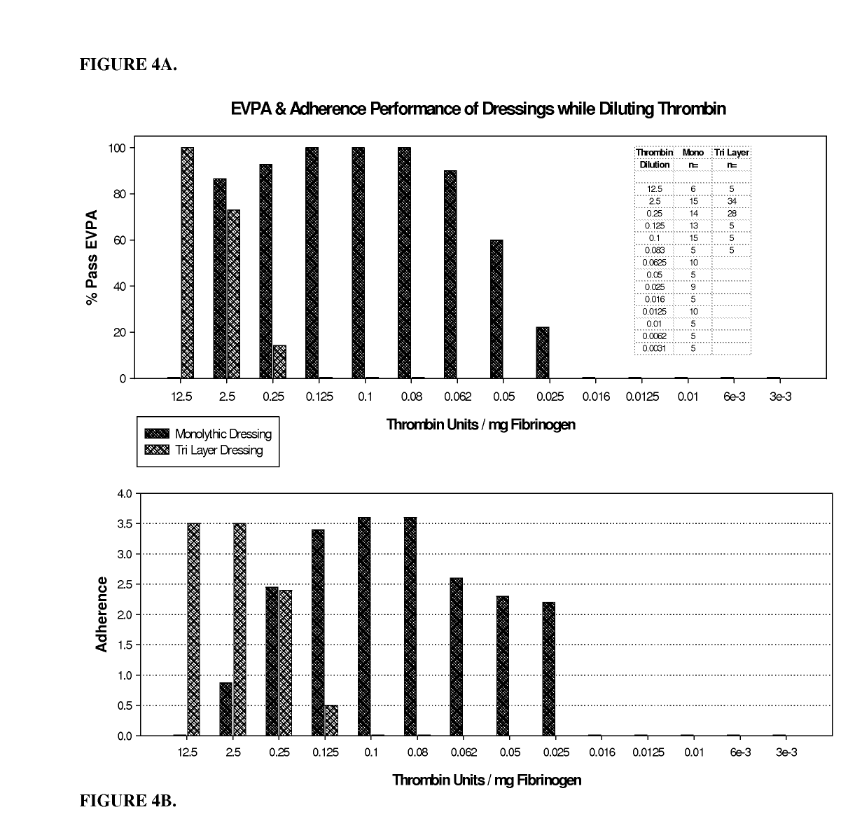

FIGS. 5A and 5B are graphs showing the performance characteristics of frozen compositions stored at −80° C. as shown in Example 13.

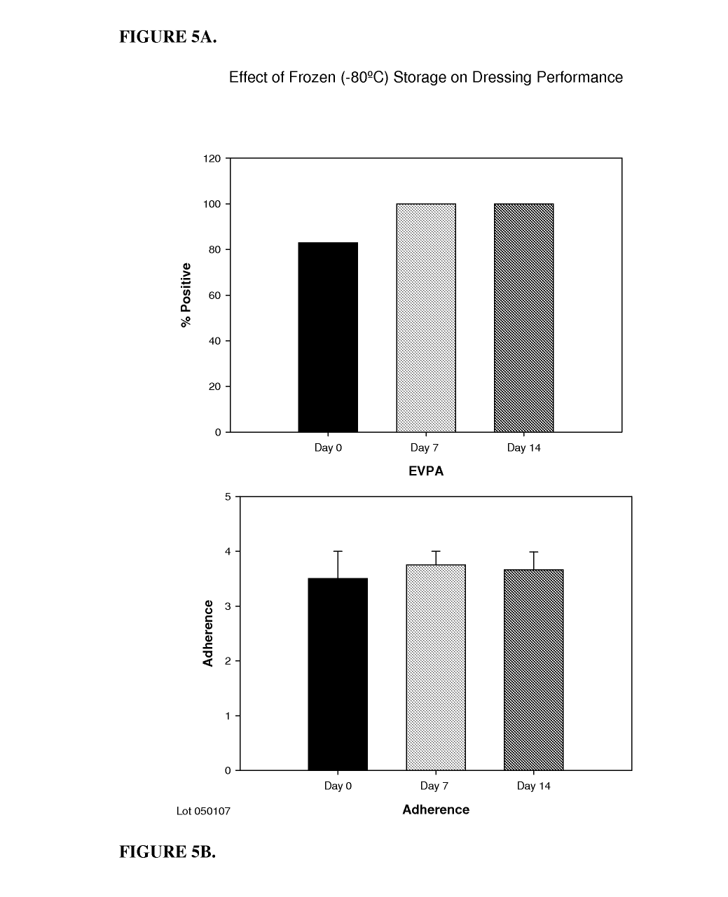

FIG. 6 is a diagram showing Structural Changes During Conversion of Fibrinogen to Fibrin.

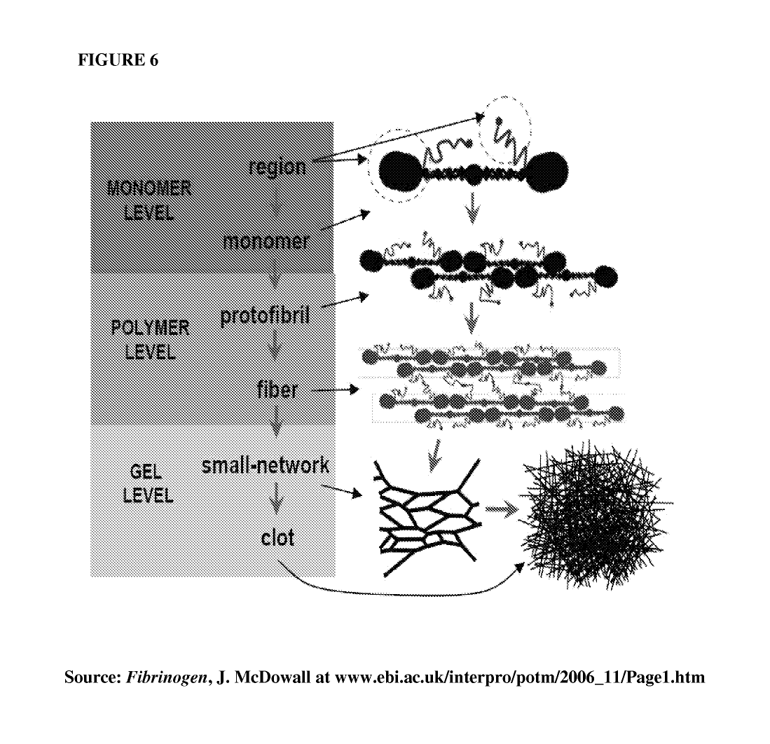

FIG. 7 is a NativePAGE™ Gel of Supernatants from 5 Minute Incubation Conditions.

FIG. 8 is a NativePAGE™ Gel of Supernatants from 30 Minute Incubation Conditions.

FIG. 9 is a NativePAGE™ Gel of Controls Prepared 0.9% NaCl Solution.

FIG. 10 is a NativePAGE™ Gel of Controls Prepared in ODS.

FIG. 11 is a NativePAGE™ Gel of Controls Prepared in ODS and Heated.

FIG. 12 is a Non-Reduced 4% Tris-Glycine Gel of Controls Prepared with Heating.

FIG. 13 is a Non-Reduced 4% Tris-Glycine Gel of Controls Prepared in ODS with Heating.

FIG. 14 is a Non-Reduced Gels of Supernatants from 5 Minute Incubation Conditions.

FIG. 15 is a Non-Reduced Gels of Supernatants from 30 Minute Incubation Conditions.

FIG. 16 is a SDS-PAGE Gel of Unclotted Dressing Samples.

FIG. 17 SDS-PAGE Gel of Clotted Dressing Samples.

### DETAILED DESCRIPTION OF PREFERRED EMBODIMENTS

Unless defined otherwise, all technical and scientific terms used herein have the same meaning as is commonly understood by one of skill in the art to which this invention belongs. All patents and publications mentioned herein are incorporated by reference.

As used herein, use of a singular article such as “a,” “an,” and “the” is not intended to excluded pluralities of the article's object unless the context clearly and unambiguously dictates otherwise.

“About” as used herein means within 10% of a stated number.

“Substantially” as used herein means something done with the intent of the action being complete but allowing for 5% variance. For example substantially unreacted, means intended to have no reaction, but allowing up to 5% reaction to have occurred.

“Patient” as used herein refers to human or animal individuals in need of medical care and/or treatment.

As used herein, “fibrin” refers to fibrin polymers, predominantly cross-linked via their gamma chains, that are substantially insoluble under physiological conditions.

As used herein, “Stroetmann fibrin” refers to a product of reacting fibrinogen and thrombin as described in U.S. Pat. No. 4,442,655 that is soluble under physiological conditions.

“Wound” as used herein refers to any damage to any tissue of a patient which results in the loss of blood from the circulatory system and/or any other fluid from the patient's body. The tissue may be an internal tissue, such as an organ or blood vessel, or an external tissue, such as the skin. The loss of blood may be internal, such as from a ruptured organ, or external, such as from a laceration. A wound may be in a soft tissue, such as an organ, or in hard tissue, such as bone. The damage may have been caused by any agent or source, including traumatic injury, infection or surgical intervention.

“Resorbable material” as used herein refers to a material that is broken down spontaneously and/or by the mammalian body into components which are consumed or eliminated in such a manner as not to interfere significantly with wound healing and/or tissue regeneration, and without causing any significant metabolic disturbance.

“γ-γ dimer” as used herein, means covalently cross-linked fibrinogen γ chains. Since the resulting structure has a higher apparent molecular weight then single γ chains, and can be separated from the α and β chains by molecular weight, the relative amount of γ-γ versus free γ chains in a sample can be determined. Further, since the formation of γ-γ dimers from γ chains occurs late in the transformation of fibrinogen to insoluble fibrin, it can be used to quantify the amount of fibrin in a sample.

“Stability” as used herein refers to the retention of those characteristics of a material that determine activity and/or function.

“Suitable” as used herein is intended to mean that a material does not adversely affect the stability of the dressings or any component thereof.

“Binding agent” as used herein refers to a compound or mixture of compounds that improves the adherence and/or cohesion of the components of the haemostatic layer(s) of the dressings.

“Solubilizing agent” as used herein refers to a compound or mixture of compounds that improves the dissolution of a protein or proteins in aqueous solvent.

“Filler” as used herein refers to a compound or mixture of compounds that provide bulk and/or porosity to the haemostatic layer(s) of a dressing.

“Release agent” as used herein refers to a compound or mixture of compounds that facilitates removal of a dressing from a manufacturing mold.

“Foaming agent” as used herein refers to a compound or mixture of compounds that produces gas when hydrated under suitable conditions.

“Solid” as used herein is intended to mean that the dressing will not substantially change in shape or form when placed on a rigid surface, wound-facing side down, and then left to stand at room temperature for 24 hours.

“Frozen” as used herein is intended to mean that the composition will not substantially change in shape or form when placed on a rigid surface, wound-facing side down, and then left to stand at −40° C. for 24 hours, but will substantially change in shape or form when placed on a rigid surface, wound-facing side down, and then left at room temperature for 24 hours. Thus, in the context of the present invention, a “solid” dressing is not “frozen” and a “frozen” composition is not “solid”.

As used herein, “consisting essentially of” is intended to mean that the fibrinogen component and the fibrinogen activator are the only necessary and essential ingredients of the haemostatic layer(s) of the solid dressing when it is used as intended to treat wounded tissue. Accordingly, the haemostatic layer may contain other ingredients in addition to the fibrinogen component and the fibrinogen activator as desired for a particular application, but these other ingredients are not required for the solid dressing to function as intended under normal conditions, i.e. these other ingredients are not necessary for the fibrinogen component and fibrinogen activator to react and form enough fibrin to reduce the flow of blood and/or fluid from normal wounded tissue when that dressing is applied to that tissue under the intended conditions of use. If, however, the conditions of use in a particular situation are not normal, for example the patient is a hemophiliac suffering from Factor XIII deficiency, then the appropriate additional components, such as Factor XIII/XIIIa or some other transaminase, may be added to the haemostatic layer(s) without deviating from the spirit of the present invention. Similarly, the solid dressing of the present invention may contain one or more of these haemostatic layers as well as one or more other layers, such as one or more support layers (e.g. a backing material or an internal support material) and release layers.

As used herein, “moisture content” refers to the amount freely-available water in the dressing. “Freely-available” means the water is not bound to or complexed with one or more of the non-liquid components of the dressing. The moisture content referenced herein refers to levels determined by procedures substantially similar to the FDA-approved, modified Karl Fischer method (Meyer and Boyd, Analytical Chem., 31:215-219, 1959; May et al., J. Biol. Standardization, 10:249-259, 1982; Centers for Biologics Evaluation and Research, FDA, Docket No. 89D-0140, 83-93; 1990) or by near infrared spectroscopy. Suitable moisture content(s) for a particular solid dressing may be determined empirically by one skilled in the art depending upon the intended application(s) thereof.

According to one embodiment of the invention, a haemostatic layer is one that comprises both a fibrinogen component and a fibrinogen activator. For example, one embodiment of the invention utilizes fibrinogen and thrombin. In forming a haemostatic layer, a mold is placed in a −80° C. freezer for about 60 minutes to cool the mold. The mold is then placed on a copper plate, which is situated in dry ice, to maintain the cold temperature. Fibrinogen and thrombin are separately formulated into aqueous solution, each having a pH of about 7.4±0.1 and then each solution is cooled to about 4° C.±4° C. The fibrinogen and thrombin solutions are then simultaneously dispensed into the mold and allowed to freeze. The then frozen mold and fibrinogen and thrombin are then placed into a −80° C. freezer for about two hours. Subsequently, the frozen mold and now mixture of fibrinogen and thrombin are placed into a pre-cooled lypohilizer and dried.

In other embodiments, the cooled fibrinogen and cooled thrombin solutions may be combined together, and then added to the pre-cooled mold before frozen. In each embodiment, the mixture of the fibrinogen and thrombin a substantially homogeneous aqueous mixture of the components, allowing for molecules of fibrinogen and molecules of thrombin to be together, substantially unreacted in an aqueous medium.

A mixture of a fibrinogen component and a fibrinogen activator and water may be frozen and stored at a reduced temperature for at least 24 hours. Typically, a reduced temperatures is about −80° C., however temperatures from about −196° C. to about 0° C. may be sufficient. Such compositions are particularly useful for preparing the haemostatic layer(s) of the inventive solid dressings.

According to certain embodiments of the present invention, the formation of the haemostatic layer of the solid dressing is formed or cast as a single piece, wherein the layer comprises the fibrinogen component, fibrinogen activator, that are in solution with an aqueous medium before being dried to form the solid dressing. According to certain other embodiments of the present invention, the haemostatic layer is made or formed into or from a single source, e.g. an aqueous solution containing a mixture of the fibrinogen and the fibrinogen activator is pre-mixed before freezing. According to these embodiments, the haemostatic layer may be formed by introducing a liquid aqueous mixture of the fibrinogen component and the fibrinogen activator into a suitable vessel, such as a mold or the like, and then reducing the temperature to form a substantially homogeneous frozen aqueous mixture of the fibrinogen component and the fibrinogen activator. With each of these embodiments of the present invention, the haemostatic layer is substantially homogeneous throughout.

According to certain preferred embodiments, the haemostatic layer(s) of the solid dressing may also contain a binding agent to facilitate or improve the adherence of the layer(s) to one another and/or to any support layer(s). Illustrative examples of suitable binding agents include, but are not limited to, sucrose, mannitol, sorbitol, gelatin, hyaluron and its derivatives, such as hyaluronic acid, maltose, povidone, starch, chitosan and its derivatives, and cellulose derivatives, such as carboxymethylcellulose, as well as mixtures of two or more thereof.

The haemostatic layer(s) of the solid dressing may also optionally contain one or more suitable fillers, such as sucrose, lactose, maltose, silk, fibrin, collagen, albumin, polysorbate (Tween™), chitin, chitosan and its derivatives (e.g. NOCC-chitosan), alginic acid and salts thereof, cellulose and derivatives thereof, proteoglycans, hyaluron and its derivatives, such as hyaluronic acid, glycolic acid polymers, lactic acid polymers, glycolic acid/lactic acid co-polymers, and mixtures of two or more thereof.

The haemostatic layer of the solid dressing may also optionally contain one or more suitable solubilizing agents, such as sucrose, lactose, maltose, dextrose, mannose, trehalose, mannitol, sorbitol, albumin, hyaluron and its derivatives, such as hyaluronic acid, sorbate, polysorbate (Tween™), sorbitan (SPAN™) and mixtures of two or more thereof.

The haemostatic layer of the solid dressing may also optionally contain one or more suitable foaming agents, such as a mixture of a physiologically acceptable acid (e.g. citric acid or acetic acid) and a physiologically suitable base (e.g. sodium bicarbonate or calcium carbonate). Other suitable foaming agents include, but are not limited to, dry particles containing pressurized gas, such as sugar particles containing carbon dioxide (see, e.g., U.S. Pat. No. 3,012,893) or other physiologically acceptable gases (e.g. Nitrogen or Argon), and pharmacologically acceptable peroxides. Such a foaming agent may be introduced into the aqueous mixture of the fibrinogen component and the fibrinogen activator, or may be introduced into an aqueous solution of the fibrinogen component and/or an aqueous solution of the fibrinogen activator prior to mixing.

The haemostatic layer(s) of the solid dressing may also optionally contain a suitable source of calcium ions, such as calcium chloride, and/or a fibrin cross-linker, such as a transaminase (e.g. Factor XIII/XIIIa) or glutaraldehyde.

The haemostatic layer of the solid dressing is preferably prepared by mixing aqueous solutions of the fibrinogen component and the fibrinogen activator under conditions which minimize the activation of the fibrinogen component by the fibrinogen activator. The mixture of aqueous solutions is then subjected to a process such as lyophilization or free-drying to reduce the moisture content to the desired level, i.e. to a level where the dressing is solid and therefore will not substantially change in shape or form upon standing, wound-facing surface down, at room temperature for 24 hours. Similar processes that achieve the same result, such as drying, spray-drying, vacuum drying and vitrification, may also be employed.

The process of mixing the fibrinogen and thrombin components together in solution provides a significant benefit for creation of a substantially homogeneous haemostatic bandage. Molecules in solution are much closer in proximity as compared to particles of powdered fibrinogen and powdered thrombin. Furthermore, particles of fibrinogen and to a lesser extent thrombin, form hydrogels, which further prevent the reaction between the fibrinogen and thrombin. These factors also contribute to non-homogenous mixing of the fibrinogen free (in solution) fibrinogen and thrombin molecules resulting in inconsistent and degraded performance. In contrast, utilizing the present invention, when in solution, the fibrinogen and thrombin can react homogeneously to create strong, homogeneous clot. When utilized on a wound surface, this provides for a significant improvement over prior bandages in clotting an open wound.

Another embodiment of the Invention would comprise adding one or more of the reactive components (i.e. fibrinogen, thrombin, factor XIII etc.) to an aqueous medium in an amount that exceeded their solubility in that medium, and which was cooled to a sufficiently low temperature to prevent them from reacting prior to freezing. Under some embodiments of this invention, the quantity of one or more reactants may not be above their ultimate solubility in the aqueous medium, however the quantity would take longer to dissolve than required to freeze the resulting aqueous mixture. In still other embodiments of this invention, the quantity of one or more reactants may not be above their ultimate solubility in the aqueous medium at a higher temperature, such as room or body temperatures (approximately 20 and 37° C. respectively), however the quantity would exceed the solubility limit in the chilled aqueous mixture.

According to certain preferred embodiments of the present invention, the solid dressing is manufactured using a mold. According to these embodiments, the solid dressings may also optionally further comprise a release layer in addition to the haemostatic layer(s) and support layer(s). As used herein, a “release layer” refers to a layer containing one or more agents (“release agents”) which promote or facilitate removal of the solid dressing from a mold in which it has been manufactured. A preferred such agent is sucrose, but other suitable release agents include gelatin, polysorbate, sorbitan, lactose, maltose, trehalose, sorbate, glucose, hyaluron and its derivatives, such as hyaluronic acid, mannitol, sorbitol and glucose. Such a release layer is preferably placed or formed in the mold prior to introducing the liquid aqueous mixture or the solutions of fibrinogen component and fibrinogen activator.

Alternatively, such one or more release agents may be contained in the haemostatic layer. According to these embodiments, the release agent may introduced into the liquid aqueous mixture prior to or during introduction of the liquid aqueous mixture into the mold. The release agent may also be introduced into the solution of fibrinogen component and/or the solution of fibrinogen activator prior to or during formation of the liquid aqueous mixture.

The aqueous mixture of the fibrinogen component and the fibrinogen activator may be performed in any suitable vessel. According to certain preferred embodiments, the vessel used for mixing is the mold in which the liquid aqueous mixture is to be subsequently frozen. According to such embodiments, separate liquid aqueous solutions of the fibrinogen component and the fibrinogen activator are simultaneously introduced into the mold, thereby causing the two solutions to mix. Alternatively, a single liquid aqueous solution of the fibrinogen component and the fibrinogen activator may be prepared in a vessel and then subsequently introduced into the mold.

The size and geometry of a given mold may be determined empirically by one skilled in the art depending upon the desired size and shape of the solid dressing being produced. Suitable materials for a mold include, but are not limited to, polymers such as polyvinyl chloride (PVC), Glycol-modified polyethlylenetetrapthalate (PETG) and polyethylene. Other suitable materials include metals, such as stainless steel, paper, cardboard and waterproofed paper or cardboard. The mold may also be fabricated from a rapidly dissolving material that is solid at the temperatures at which the mold is kept and/or from a material that is capable of being lyophilized into a solid state.

According to the methods of the present invention, formation of a liquid aqueous mixture of the fibrinogen component and the fibrinogen activator is performed at a temperature that is sufficiently low to inhibit the activation of the fibrinogen component by the fibrinogen activator.

For a properly functioning fibrinogen/thrombin-based solid dressing, hydration should normally be completed within a few seconds and require nothing more than applying water (or some aqueous solution) onto the dressing. This solution could be blood or another bodily fluid from an injury site that the dressing is applied to, or it may be from some external source, such as a saline or other physiologically acceptable aqueous liquid applied to the dressing while it is on the wound to be treated. Longer hydration times, i.e. generally greater than 5 seconds, may impede the dressing's performance as portions of the dressing may be lost or shed into the fluids which will continue to freely flow prior to formation of sufficient cross-linked fibrin. Given the potentially fatal consequences of continued bleeding, any delay in dressing hydration during use is highly undesirable.

Activation of the fibrinogen component by the fibrinogen activator may be determined by any suitable method known and available to those skilled in the art. For example, the formation of fibrin from activation of the fibrinogen component can be detected visually by noting deviations from a homogenous surface appearance in the resulting solid dressing. Solid, smooth and glossy “sheets” on the surface of solid dressings are also signs of fibrin, as is excessive curling of the edges. Moreover, once hydrated, dressings with a significant amount of fibrin usually have a mottled and distinctly non-homogenous appearance.

Preferably, activation of the fibrinogen component may be assessed by various biochemical assays, such as the method described in U.S. Patent Application Publication No. US 2006/0155234 A1. According to this assay, the conversion of the fibrinogen γ chains to cross-linked γ-γ dimers may be used as an indication of the activation of the fibrinogen component by the fibrinogen activator (the proportion of γ chain that is converted to γ-γ dimer being related to the amount of fibrinogen activated). However, some less pure fibrinogen products contain some measurable amounts of γ-γ dimer. The presence of these γ-γ dimers in the material used to fabricate the haemostatic layer does not necessarily indicate the conversion of the fibrinogen component by the fibrinogen activator in the creation of the haemostatic layer.

Other biochemical assays could assess changes in the other component chains of fibrinogen, such as the conversion of the Aα chain into free α chain and fibrinopeptide A or the conversion of the Bβ chain into free β chain and fibrinopeptide B. These changes can be monitored by gel electrophoresis in a similar manner to the γ to γ-γ conversion described in U.S. Patent Application Publication No. US 2006/0155234 A1.

Preferred liquid aqueous mixtures prepared using the inventive processes generally contain no detectable to γ-γ dimer or having about less than about 1% γ-γ dimer, although it may be acceptable in certain circumstances for the dressing to contain from about 1% to about 9% γ-γ dimer or less than about 1%, 2%, 3%, 4%, 5%, 6%, 7%, 8%, or 9% γ-γ dimer. In certain circumstances, the presence of more than 9% γ-γ dimer is also acceptable.

The presence of fibrin, as measured by the amount of γ-γ dimer in it prior to application to wounded tissue, limits the ability of the haemostatic layer to adhere to an open wound. Fibrin, particularly insoluble fibrin, being the reaction product produced by fibrinogen plus thrombin and Factor XIII, does not allow for significant γ chain formation with the wound surface itself, and thus too much fibrin limits the ability of a bandage to adhere to the wound to stop bleeding. A measure of the change in the percentage of γ-γ dimer is an additional way to characterize the amount of reaction, by comparing the % γ-γ dimer in the fibrinogen and then thereafter, once mixed with thrombin in either the liquid, frozen solid, or lyophilized solid bandage. Having less than 10% increase in % γ-γ dimer is important to ensure sufficient unreacted fibrinogen and thrombin to support a strong clot with the body.

Accordingly, a liquid, or frozen liquid mixture having from about 1% to about 38% γ-γ dimer is acceptable, or less than 10% γ-γ dimer is acceptable. Similarly, a solid lyophilized product of the invention described herein having about 1% to about 38% or about 9% to about 38%, or about 9% to about 29%, or any amount in between, is acceptable in certain circumstances, preferably less than 10% γ-γ dimer, less than about 5% γ-γ dimer, or less than about 3% γ-γ dimer.

Similarly, other methods of characterizing the bandage count the amounts of free α chain and fibrinopeptide A and free β chain a fibrinopeptide B. Accordingly, preferred liquid aqueous mixtures may contain up to 60% free α or β chains and yet still perform acceptably. Particularly preferred aqueous mixtures will contain less than 60% free α or β chain, more preferably less than 50%, and even more preferably less than 40%, less than 30%, less than 20% or less than 10% free α or β chain. According to certain preferred embodiments, these values do not significantly change over time when the liquid aqueous mixture is maintained at a suitable temperature, preferably at or below 4° C.±2° C.

The temperature for preparing the liquid aqueous mixtures is sufficient to inhibit the activation of the fibrinogen component by the fibrinogen activator. Preferably, this temperature is at or below about 2° C. to about 8° C., or more preferably at about 4° C.±2° C. Any suitable method may be used to achieve the desired temperature for preparing the liquid aqueous mixture.

According to certain preferred embodiments, the vessel or mold is cooled to a desired temperature by placing it in an environment having a temperature at or below the desired temperature. Preferably, the vessel or mold is cooled to a temperature substantially below the desired temperature for preparing the liquid aqueous mixture. According to a particularly preferred embodiment, the vessel or mold is cooled by placing it in an environment having a temperature of about −80° C. for a sufficient time. This allows the vessel to be significantly below the temperature of any liquid being added to the vessel or mold and facilitates a rapid freezing of the liquid fibrinogen component and fibrinogen activator mixture.

According to other embodiments, the aqueous solution of the fibrinogen component and the aqueous solution of the fibrinogen activator are cooled to a desired temperature before preparing the liquid aqueous mixture. Such cooling may be achieved by any suitable method, such as placing vessels containing the aqueous solutions on ice.

Once the liquid aqueous mixture has been prepared, it may be stored at a suitable temperature or it may be used directly to prepare the frozen aqueous mixture of the present invention. Preferably, the liquid aqueous mixture is used directly to prepare the frozen aqueous mixture.

According to certain preferred embodiments of the present invention, the liquid aqueous mixture is subsequently cooled to a temperature where it becomes a frozen aqueous mixture of the fibrinogen component and the fibrinogen activator. Such a frozen aqueous mixture may be used directly, or it may be stored at a suitable temperature.

The liquid aqueous mixture may be cooled to the requisite temperature using any of the methods and techniques known and available to those skilled in the art. For example, the liquid aqueous mixture may be introduced into a second vessel or mold which has been cooled to a temperature sufficient to cause the liquid aqueous mixture to freeze. Alternatively, the liquid aqueous mixture in the vessel or mold may be placed in an environment having a temperature sufficient to cause the liquid aqueous mixture to freeze. Such an environment could include, for example, a freezer set to a predetermined temperature, such as −5° C., −10° C., −15° C., −20° C., −25° C., −30° C., −40° C., −50° C. or −80° C., or temperatures in between. The mold may be placed such that one or more surfaces thereof is in intimate contact with a surface that has been and/or is cooled to a desired temperature. Alternatively, the mold or vessel containing the liquid aqueous solution can be placed directly on dry ice (−78° C.) or into a suitable cooling bath, such as dry ice/acetone, dry ice/liquid nitrogen or liquid nitrogen alone. The mold or vessel may also be placed in a stream of nitrogen gas produced by the evaporation of liquid nitrogen or other suitable cryogenic gas coolant. In this case, it may be desirable for the gaseous stream to contact a single side of the mold to be cooled. In a more preferred embodiment, such a stream could be directed to two or more sides of the object to be cooled.

Preferred frozen aqueous mixtures prepared using the inventive processes generally contain no detectable to γ-γ dimer, although it may be acceptable in certain circumstances for the mixture to contain about 1% to about 9% γ-γ dimer or even about 38% γ-γ dimer. Similarly, preferred frozen aqueous mixtures may contain up to about 57% free α chain and yet still perform acceptably. Particularly preferred frozen aqueous mixtures will contain less than 57% free α chain, more preferably less than 46%, and even more preferably less than 46%, less than 31%, less than 20%, less than 16% or less than 10% free α chain. According to certain preferred embodiments, these values do not significantly change over time.

The frozen aqueous mixture may be stored or used directly, either to prepare solid dressings or to treat wounded tissue. Certain embodiments of the present invention are directed to these frozen aqueous mixtures, and to their use either for the preparation of solid dressings or for the treatment of wounded tissue.

A step in the formation of the solid haemostatic bandage in some embodiments is the drying process. Through the drying process, water is removed from the frozen aqueous solution to form a dried haemostatic layer. Subsequent to the removal of this water, in certain embodiments of the present invention, some moisture present is still advantageous as higher moisture contents are associated with more flexible solid dressings. Thus, in solid dressings intended for extremity wounds, it may be preferred to have a moisture content of at least 6% and even more preferably in the range of 6% to 44%.

Similarly, in other embodiments of the present invention, lower moisture contents are associated with more rigid solid dressings. Thus, in solid dressings intended for flat wounds, such as wounds to the abdomen or chest, it may be preferred to have a moisture content of less than 6% and even more preferably in the range of 1% to 6%. Thus, typically bandages have from about 1% to about 44% moisture content.

Accordingly, illustrative examples of suitable moisture contents for solid dressings include, but are not limited to, the following (each value being ±0.9%): less than 53%; less than 44%; less than 28%; less than 24%; less than 16%; less than 12%; less than 6%; less than 5%; less than 4%; less than 3%; less than 2.5%; less than 2%; less than 1.4%; between 0 and 12%, non-inclusive; between 0 and 6%; between 0 and 4%; between 0 and 3%; between 0 and 2%; between 0 and 1%; between 1 and 16%; between 1 and 11%; between 1 and 8%; between 1 and 6%; between 1 and 4%; between 1 and 3%; between 1 and 2%; and between 2 and 4%.

The fibrinogen component in the haemostatic layer(s) of the solid dressings may be any suitable fibrinogen known and available to those skilled in the art. The fibrinogen component may also be a functional derivative or metabolite of a fibrinogen, such the fibrinogen α, β and/or γ chains, soluble fibrin I or fibrin II, or a mixture of two or more thereof. A specific fibrinogen (or functional derivative or metabolite) for a particular application may be selected empirically by one skilled in the art. As used herein, the term “fibrinogen” is intended to include mixtures of fibrinogen and small mounts of Factor XIII/Factor XIIIa, or some other such transaminase. Such small amounts are generally recognized by those skilled in the art as usually being found in mammalian fibrinogen after it has been purified according to the methods and techniques presently known and available in the art, and typically range from 0.1 to 20 Units/mL.

Preferably, the fibrinogen employed as the fibrinogen component of the solid dressing is a purified fibrinogen suitable for introduction into a mammal. Typically, such fibrinogen is a part of a mixture of human plasma proteins which include Factor XIII/XIIIa and have been purified to an appropriate level and virally inactivated. A preferred aqueous solution of fibrinogen for preparation of a solid dressing contains around 37.5 mg/mL fibrinogen at a pH of about 7.4±0.1. However, suitable fibrinogen may be at a pH of about 6.5 to about 7.8. Suitable fibrinogen for use as the fibrinogen component has been described in the art, e.g. U.S. Pat. No. 5,716,645, and similar materials are commercially available, e.g. from sources such as Sigma-Aldrich, Enzyme Research Laboratories, Haematologic Technologies and Aniara.

Preferably, the fibrinogen component is present in an amount of from about 1.5 to about 13.0 mg (±0.9 mg) of fibrinogen per square centimeter of solid dressing, and more preferably from about 3.0 to about 13.0 mg/cm². Greater or lesser amounts, however, may be employed depending upon the particular application intended for the solid dressing. For example, according to certain embodiments where increased adherence is desired, the fibrinogen component is present in an amount of from about 11.0 to about 13.0 mg (±0.9 mg) of fibrinogen per square centimeter of solid dressing. Likewise, according to certain embodiments which are intended for treating low pressure-containing vessels, lower levels of the fibrinogen component may be employed.

The fibrinogen activator employed in the haemostatic layer(s) of the solid dressing may be any of the substances or mixtures of substances known by those skilled in the art to convert fibrinogen into fibrin. Illustrative examples of suitable fibrinogen activators include, but are not limited to, the following: thrombins, such as human thrombin or bovine thrombin, and prothrombins, such as human prothrombin or prothrombin complex concentrate (a mixture of Factors II, VII, IX and X); snake venoms, such as batroxobin, reptilase (a mixture of batroxobin and Factor XIIIa), bothrombin, calobin, fibrozyme, and enzymes isolated from the venom of Bothrops jararacussu; and mixtures of any two or more of these. See, e.g., Dascombe et al., Thromb. Haemost. 78:947-51 (1997); Hahn et al., J. Biochem. (Tokyo) 119:835-43 (1996); Fortova et al., J. Chromatogr. S. Biomed. Appl. 694:49-53 (1997); and Andriao-Escarso et al., Toxicon. 35: 1043-52 (1997).

Preferably, the fibrinogen activator is a thrombin. More preferably, the fibrinogen activator is a mammalian thrombin, although bird and/or fish thrombin may also be employed in appropriate circumstances. While any suitable mammalian thrombin may be used in the solid dressing, the thrombin employed in the haemostatic layer is preferably a lyophilized mixture of human plasma proteins which has been sufficiently purified and virally inactivated for the intended use of the solid dressing. Suitable thrombin is available commercially from sources such as Sigma-Aldrich, Enzyme Research Laboratories, Haematologic Technologies and Biomol International. A particularly preferred aqueous solution of thrombin for preparing a solid dressing contains thrombin at a potency of between 10 and 2000±50 International Units/mL, and more preferred at a potency of 25±2.5 International Units/mL. Other constituents may include albumin (generally about 0.1 mg/mL) and glycine (generally about 100 mM±0.1 mM). The pH of this particularly preferred aqueous solution of thrombin is generally in the range of 6.5-7.8, and preferably 7.4±0.1.

In addition to the haemostatic layer(s), the solid dressing may optionally further comprise one or more support layers. As used herein, a “support layer” refers to a material that sustains or improves the structural integrity of the solid dressing and/or the fibrin clot formed when such a dressing is applied to wounded tissue.

According to certain preferred embodiments of the present invention the support layer comprises a backing material on the side of the dressing opposite the side to be applied to wounded tissue. Such a backing material may be affixed with a physiologically-acceptable adhesive or may be self-adhering (e.g. by having a sufficient surface static charge). The backing material may comprise one or more resorbable materials or one or more non-resorbable materials or mixtures thereof. Preferably, the backing material is a single resorbable material.

Any suitable resorbable material known and available to those skilled in the art may be employed in the present invention. For example, the resorbable material may be a proteinaceous substance, such as silk, fibrin, keratin, collagen and/or gelatin. Alternatively, the resorbable material may be a carbohydrate substance, such as alginates, chitin, cellulose, proteoglycans (e.g. poly-N-acetyl glucosamine), glycolic acid polymers, lactic acid polymers, or glycolic acid/lactic acid co-polymers. The resorbable material may also comprise a mixture of proteinaceous substances or a mixture of carbohydrate substances or a mixture of both proteinaceous substances and carbohydrate substances. Specific resorbable material(s) may be selected empirically by those skilled in the art depending upon the intended use of the solid dressing.

According to certain preferred embodiments of the present invention, the resorbable material is a carbohydrate substance. Illustrative examples of particularly preferred resorbable materials include, but are not limited to, the materials sold under the trade names VICRYL™ (a glycolic acid/lactic acid copolymer) and DEXON™ (a glycolic acid polymer).

Any suitable non-resorbable material known and available to those skilled in the art may be employed as the backing material. Illustrative examples of suitable non-resorbable materials include, but are not limited to, plastics, silicone polymers, paper and paper products, latex, gauze and the like.

According to other preferred embodiments, the support layer comprises an internal support material. Such an internal support material is preferably fully contained within a haemostatic layer of the solid dressing, although it may be placed between two adjacent haemostatic layers in certain embodiments. As with the backing material, the internal support material may be a resorbable material or a non-resorbable material, or a mixture thereof, such as a mixture of two or more resorbable materials or a mixture of two or more non-resorbable materials or a mixture of resorbable material(s) and non-resorbable material(s).

According to still other preferred embodiments, the support layer may comprise a front support material on the wound-facing side of the dressing, i.e. the side to be applied to wounded tissue. As with the backing material and the internal support material, the front support material may be a resorbable material or a non-resorbable material, or a mixture thereof, such as a mixture of two or more resorbable materials or a mixture of two or more non-resorbable materials or a mixture of resorbable material(s) and non-resorbable material(s).

A solid dressing may comprise one haemostatic layer or multiple haemostatic layers. Each haemostatic layer comprises both a fibrinogen component and a fibrinogen activator. For example, each layer would comprise both fibrinogen and thrombin. According to still other preferred embodiments, the solid dressing comprises both a backing material and an internal support material in addition to the haemostatic layer(s), i.e. the solid dressing comprises two support layers in addition to the haemostatic layer(s). According to still other preferred embodiments, the solid dressing comprises both a front support material and an internal support material in addition to the haemostatic layer(s). According to still other preferred embodiments, the solid dressing comprises a backing material, a front support material and an internal support material in addition to the haemostatic layer(s).

According to certain embodiments of the present invention, particularly where the solid dressing is manufactured using a mold, the solid dressings may also optionally further comprise a release layer in addition to the haemostatic layer(s) and support layer(s). As used herein, a “release layer” refers to a layer containing one or more agents (“release agents”) which promote or facilitate removal of the solid dressing from a mold in which it has been manufactured. A preferred such agent is sucrose, but other suitable release agents include gelatin, hyaluron and its derivatives, including hyaluronic acid, mannitol, sorbitol and glucose. Alternatively, such one or more release agents may be contained in the haemostatic layer.

The various layers of the inventive dressings may be affixed to one another by any suitable means known and available to those skilled in the art. For example, a physiologically-acceptable adhesive may be applied to a backing material (when present), and the haemostatic layer(s) subsequently affixed thereto.

In certain embodiments of the present invention, the physiologically-acceptable adhesive has a shear strength and/or structure such that the backing material can be separated from the fibrin clot formed by the haemostatic layer after application of the dressing to wounded tissue. In other embodiments, the physiologically-acceptable adhesive has a shear strength and/or structure such that the backing material cannot be separated from the fibrin clot after application of the bandage to wounded tissue.

Suitable fibrinogens and suitable fibrinogen activators for the haemostatic layer(s) of the solid dressing may be obtained from any appropriate source known and available to those skilled in the art, including, but not limited to, the following: from commercial vendors, such as Sigma-Aldrich and Enzyme Research Laboratories; by extraction and purification from human or mammalian plasma by any of the methods known and available to those skilled in the art; from supernatants or pastes derived from plasma or recombinant tissue culture, viruses, yeast, bacteria, or the like that contain a gene that expresses a human or mammalian plasma protein which has been introduced according to standard recombinant DNA techniques; and/or from the fluids (e.g. blood, milk, lymph, urine or the like) of transgenic mammals (e.g. goats, sheep, cows) that contain a gene which has been introduced according to standard transgenic techniques and that expresses the desired fibrinogen and/or desired fibrinogen activator.

According to certain preferred embodiments of the present invention, the fibrinogen is a mammalian fibrinogen such as bovine fibrinogen, porcine fibrinogen, ovine fibrinogen, equine fibrinogen, caprine fibrinogen, feline fibrinogen, canine fibrinogen, murine fibrinogen or human fibrinogen. According to other embodiments, the fibrinogen is bird fibrinogen or fish fibrinogen. According to any of these embodiments, the fibrinogen may be recombinantly produced fibrinogen or transgenic fibrinogen.

According to certain preferred embodiments of the present invention, the fibrinogen activator is a mammalian thrombin, such as bovine thrombin, porcine thrombin, ovine thrombin, equine thrombin, caprine thrombin, feline thrombin, canine thrombin, murine thrombin and human thrombin. According to other embodiments, the thrombin is bird thrombin or fish thrombin. According to any of these embodiments, the thrombin may be recombinantly produced thrombin or transgenic thrombin.

As a general proposition, the purity of the fibrinogen and/or the fibrinogen activator for use in the solid dressing will be a purity known to one of ordinary skill in the relevant art to lead to the optimal efficacy and stability of the protein(s). Preferably, the fibrinogen and/or the fibrinogen activator has been subjected to multiple purification steps, such as precipitation, concentration, diafiltration and affinity chromatography (preferably immunoaffinity chromatography), to remove substances which cause fragmentation, activation and/or degradation of the fibrinogen and/or the fibrinogen activator during manufacture, storage and/or use of the solid dressing. Illustrative examples of such substances that are preferably removed by purification include: protein contaminants, such as inter-alpha trypsin inhibitor and pre-alpha trypsin inhibitor; non-protein contaminants, such as lipids; and mixtures of protein and non-protein contaminants, such as lipoproteins. However, the presence, for example of γ-γ dimer impurities in the fibrinogen does not prevent the formation of the haemostatic bandage.

The amount of the fibrinogen activator employed in the solid dressing is preferably selected to optimize both the efficacy and stability thereof. As such, a suitable concentration for a particular application of the solid dressing may be determined empirically by one skilled in the relevant art. According to certain preferred embodiments of the present invention, when the fibrinogen activator is human thrombin, the amount of human thrombin employed is between 2.50 Units/mg of fibrinogen component and 0.025 Units/mg of the fibrinogen (all values being ±0.009). Other preferred embodiments are directed to similar solid dressings wherein the amount of thrombin is between 0.250 Units/mg of fibrinogen and 0.062 Units/mg of fibrinogen and solid dressings wherein the amount of thrombin is between 0.125 Units/mg of fibrinogen and 0.080 Units/mg of fibrinogen.

The use of low amounts of thrombin is possible only because of the unique manner in which the fibrinogen and thrombin have been discovered to be combined by the Applicant. Because of the physical proximity of the fibrinogen and thrombin molecules, substantially less thrombin is necessary for substantially complete reaction of the haemostatic layer upon contact with an aqueous solution. This proximity cannot be achieved by simply taking powdered fibrinogen and powdered thrombin and mechanically combining them, as the space between the particles is large as compared to the size of the molecules. Furthermore, the formation of hydrogels limits the amounts of fibrinogen and/or thrombin available to react. Accordingly, such a mechanical mixture results in poorly combined powders that are adhered to some matrix. Accordingly, such a powdered combination requires significantly more thrombin than is used in any of the haemostatic bandages described herein.

During use of the solid dressing, the fibrinogen and the fibrinogen activator are preferably activated at the time the dressing is applied to the wounded tissue by the endogenous fluids of the patient escaping from the hemorrhaging wound. Alternatively, in situations where fluid loss from the wounded tissue is insufficient to provide adequate hydration of the protein layers, the fibrinogen component and/or the thrombin may be activated by a suitable, physiologically-acceptable liquid, optionally containing any necessary co-factors and/or enzymes, prior to or during application of the dressing to the wounded tissue.

In some embodiments of the present invention, the haemostatic layer(s) may also contain one or more supplements, such as growth factors, drugs, polyclonal and monoclonal antibodies and other compounds. Illustrative examples of such supplements include, but are not limited to, the following: fibrinolysis inhibitors, such as aprotonin, tranexamic acid and epsilon-amino-caproic acid; antibiotics, such as tetracycline and ciprofloxacin, amoxicillin, and metronidazole; anticoagulants, such as activated protein C, heparin, prostacyclins, prostaglandins (particularly (PGI₂), leukotrienes, antithrombin III, ADPase, and plasminogen activator; steroids, such as dexamethasone, inhibitors of prostacyclin, prostaglandins, leukotrienes and/or kinins to inhibit inflammation; cardiovascular drugs, such as calcium channel blockers, vasodilators and vasoconstrictors; chemoattractants; local anesthetics such as bupivacaine; and antiproliferative/antitumor drugs such as 5-fluorouracil (5-FU), taxol and/or taxotere; antivirals, such as gangcyclovir, zidovudine, amantidine, vidarabine, ribaravin, trifluridine, acyclovir, dideoxyuridine and antibodies to viral components or gene products; cytokines, such as alpha- or beta- or gamma-Interferon, alpha- or beta-tumor necrosis factor, and interleukins; colony stimulating factors; erythropoietin; antifungals, such as diflucan, ketaconizole and nystatin; antiparasitic gents, such as pentamidine; anti-inflammatory agents, such as alpha-1-anti-trypsin and alpha-1-antichymotrypsin; anesthetics, such as bupivacaine; analgesics; antiseptics; hormones; vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or antisense DNA and/or RNA); and gene therapy reagents. In other embodiments of the present invention, the backing layer and/or the internal support layer, if present, may contain one or more supplements. According to certain preferred embodiments of the present invention, the therapeutic supplement is present in an amount greater than its solubility limit in fibrin.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### EXAMPLES

The ability of the dressings to seal an injured blood vessel was determined by an ex vivo porcine arteriotomy (EVPA) performance test, which was first described in U.S. Pat. No. 6,762,336. The EVPA performance test evaluates the ability of a dressing to stop fluid flow through a hole in a porcine artery. While the procedure described in U.S. Pat. No. 6,762,336 has been shown to be useful for evaluating haemostatic dressings, it failed to replicate faithfully the requirements for success in vivo. More specifically, the procedure disclosed in U.S. Pat. No. 6,762,336 required testing at 37° C., whereas, in the real world, wounds are typically cooler than that. This decreased temperature can significantly reduce the rate of fibrin formation and its haemostatic efficacy in trauma victims. See, e.g., Acheson et al., J. Trauma 59:865-874 (2005). The test in U.S. Pat. No. 6,762,336 also failed to require a high degree of adherence of the dressing to the injured tissue. A failure mode in which fibrin forms but the dressing fails to attach tightly to the tissue would, therefore, not be detected by this test. Additionally, the pressure utilized in the procedure (200 mHg) may be exceeded during therapy for some trauma patients. The overall result of this is that numerous animal tests, typically involving small animals (such as rats and rabbits), must be conducted to accurately predict dressing performance in large animal, realistic trauma studies and in the clinical environment.

In order to minimize the amount of time and the number of animal studies required to develop the present invention, an improved ex vivo testing procedure was developed. To accomplish this, the basic conditions under which the dressing test was conducted were changed, and the severity of the test parameters was increased to include testing at lower temperatures (i.e. 29-33° C. vs. 37° C., representing the real physiologic challenge at realistic wound temperatures (Acheson et al., J. Trauma 59:865-874 (2005)), higher pressures (i.e. 250 mmHg vs. 200 mmHg), a longer test period (3 minutes vs. 2 minutes) and larger sized arterial injuries (U.S. Pat. No. 6,762,336 used an 18 gauge needle puncture, whereas the revised procedure used puncture holes ranging from 2.8 mm to 4 mm×6 mm).

In addition, a new test was derived to directly measure adherence of the dressing to the injured tissue. Both these tests showed greatly improved stringency and are thus capable of surpassing the previous ex vivo test and replacing many in vivo tests for efficacy.

The following is a list of acronyms used in the Examples below:

CFB: Complete Fibrinogen Buffer (100 mM Sodium Chloride, 1.1 mM Calcium Chloride, 10 mM Tris, 10 mM Sodium Citrate, 1.5% Sucrose, Human Serum Albumin (80 mg/g of total protein) and Tween™ 80 (animal source) 15 mg/g total protein)
CTB: Complete Thrombin Buffer (150 mM Sodium Chloride, 40 mM Calcium Chloride, 10 mM Tris and 100 mM L-Lysine with the addition of HSA at 100 ug/ml)

### ERL: Enzyme Research Laboratories

### EVPA: Ex Vivo Porcine Arteriotomy

FD: Inventive haemostatic dressing

### HSA: Human Serum Albumin

HD: A “sandwich” fibrin sealant haemostatic dressing as disclosed in U.S. Pat. No. 6,762,336
IFB: Incomplete Fibrinogen Buffer.; CFB without HSA and Tween

### PETG: Glycol-modified Polyethlylenetetrapthalate

### PPG: Polypropylene

PVC: Poly vinyl chloride
TRIS: trishydroxymethylaminomethane (2-amino-2-hydroxymethyl-1,3-propanediol)

### Example 1

Backing material (DEXON™) was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes. Fibrinogen (Enzyme Research Laboratories™) was formulated in CFB. The final pH of the fibrinogen was 7.4±0.1. The fibrinogen concentrations were adjusted to 37.5, 31.7, 25.9, 20.16, 14.4, 8.64, and 4.3 mg/ml. When 2 ml of fibrinogen was delivered into the molds, this would result in a fibrinogen dose of 13, 11, 9, 7, 5, 3 or 1.5 mg/cm². Once prepared the fibrinogen was placed on ice until use. Thrombin was formulated in CTB. The final pH of the thrombin was 7.4±0.1. The concentrations of thrombin were adjusted so that when mixed with the fibrinogen solutions as described below, the combination would produce a solution that contained 0.1 units/mg of Fibrinogen. Once prepared the thrombin was placed on ice until use. The temperature of the fibrinogen and thrombin prior to dispensing was 4° C.±2° C. Molds were removed from the −80° C. freezer and placed on a copper plate that was placed on top of dry ice. A repeat pipettor was filled with fibrinogen and second repeat pipettor was filled with thrombin. Two ml of fibrinogen and 300 micro liters of thrombin were dispensed simultaneously into each mold. Once the molds were filled they were allowed to freeze and then returned to the −80° C. freezer for at least two hours. The frozen dressings were then placed into a pre-cooled Genesis™ lyophilizer (Virtis, Gardiner, N.Y.). The chamber was sealed and the temperature equilibrated. The chamber was then evacuated and the dressings lyophilized via a primary and secondary drying cycle.

The dressings were removed from the lyophilizer, sealed in foil pouches and stored at room temperature until testing. Subsequently, the dressings were evaluated in the EVPA, Adherence and Weight Assays.

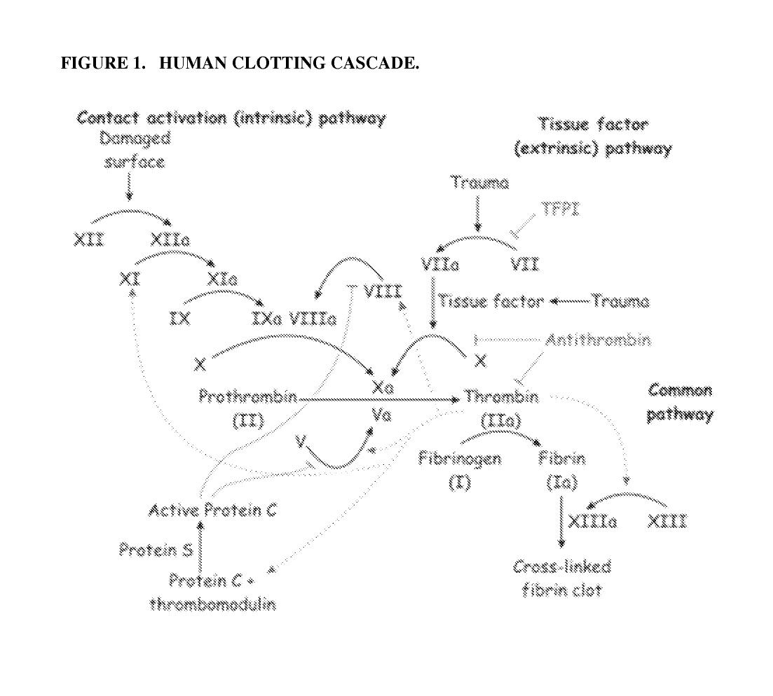

The results are given in the following Table and depicted graphically in FIGS. 1A-1C.

| | | EVPA | | | Weight | Weight |
|---|---|---|---|---|---|---|
| | | Pass/ | Peel Test | Adherence | Held | Held |
| Group | | Total | Adherence | Std Dev | (mean) (g) | Std Dev |
| 13 | mg/cm² | 6/6 | 4.0 | 0.0 | 198.0 | 12.6 |
| 11 | mg/cm² | 6/6 | 3.8 | 0.4 | 163 | 48.5 |
| 9 | mg/cm² | 5/6 | 3.0 | 0.0 | 88 | 20.0 |
| 7 | mg/cm² | 6/6 | 3.2 | 0.4 | 93 | 17.6 |
| 7 | mg/cm² | 5/6 | 3.0 | 0.0 | 94.7 | 8.2 |
| 5 | mg/cm² | 5/5 | 2.8 | 0.4 | 76 | 34.2 |
| 3 | mg/cm² | 5/5 | 2.4 | 0.5 | 48 | 27.4 |
| 1.5 | mg/cm² | 0/6 | 0.1 | 0.2 | 4.7 | 11.4 |

### Example 2

Monolithic dressings were manufactured as follows: backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes.

For all dressings, ERL fibrinogen lot 3114 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. Once prepared the thrombin was placed on ice until use. The temperature of the fibrinogen and thrombin prior to dispensing was 4° C.±2° C. Molds were removed from the −80° C. freezer and placed on a copper plate that was placed on top of dry ice. A repeat pipettor was filled with fibrinogen and second repeat pipettor was filled with thrombin. Simultaneously 2 ml of fibrinogen and 300 micro liters of thrombin were dispensed into each mold. Once the molds were filled they were returned to the −80° C. freezer for at least two hours before being placed into the freeze dryer. Dressings were then lyophilized as described above. Once complete the dressings were stored in low moisture transmission foil bags containing 5 grams of desiccant.

Tri-layer dressings were manufactured as described previously¹, using the same materials as described above. Subsequently, the dressings were placed under conditions of 100% relative humidity at 37° C. for various times in order to increase their relative moisture content to desired levels. The dressings were evaluated visually and for their handling and other physical characteristics. Following this evaluation, a sample of each of the dressings was tested to determine their moisture content. The remaining dressings were performance tested in the EVPA, Adherence and Weight Held assays.

Results

The results of the assays are given in the Tables below:

TABLE 1
Performance Data of Inventive Solid Dressings

| Exposure Time | | | Peel Test | Weight |
|---|---|---|---|---|
| to 100% | | EVPA # | Adherence | Held (g) |
| Humidity @ 37° C. | % | Pass/ | (±Std. | (mean ± Std. |
| (minutes) | Moisture | Total | Dev.) | Dev.) |
| 0 | 2.5 | 2/2 | 4.0 ± 0 | 148 ± 28.3 |
| 1 | 5.8 | 2/2 | 3.5 ± 0.71 | 123 ± 7.1 |
| 15 | 16 | 2/2 | 2.5 ± .71 | 108 ± 14.1 |
| 45 | 24 | 2/2 | 4.0 ± 0 | 168 ± 0 |
| 60 | 28 | 2/2 | 4.0 ± 0 | 273 ± 7.1 |
| 225 | 44 | 2/2 | 2 ± 0 | 58 ± 14.1 |
| 1200 | 52 | ND | ND | ND |

TABLE 2
Performance Data for Tri-layer Dressings

| Exposure Time | | | | |
|---|---|---|---|---|
| to 100% | | EVPA # | | Weight |
| Humidity @ 37° C. | % | Pass/ | Peel Test | Held (g) |
| (minutes) | Moisture | Total | Adherence | (mean) |
| 0 | 3 | 1/1 | 2.0 | 78 |
| 15 | 22 | 1/1 | 2.0 | 78 |
| 60 | 33.7 | 0/1 | 0.5 | 28 |

TABLE 3
Integrity and Handling Characteristics of Inventive Solid Dressings

| Exposure | | | | | | | |
|---|---|---|---|---|---|---|---|
| Time | | | | | During Hydration | | |
| to 100% | | | | | | Force | |
| Humidity | Prior To Hydration | | | | | Required | After |
| @ 37° C. | Surface | | | | Speed of | for | Hydration |
| (minutes) | Appearance | Curling | Integrity | Flexible | Hydration | Hydration | Appearance |
| 0 | Normal | No | Excellent | No | Normal | No | Normal |
| | (Smooth, | | (No cracks | | | | |
| | No “skin”) | | or flaking | | | | |
| | | | off) | | | | |
| 1 | Normal | ″ | Excellent | Yes | ″ | ″ | ″ |
| | (Smooth, | | (No cracks | | | | |
| | No “skin”) | | or flaking | | | | |
| | | | off) | | | | |
| 15 | Normal | ″ | Excellent | ″ | ″ | ″ | ″ |
| | (Smooth, | | (No cracks | | | | |
| | No “skin”) | | or flaking | | | | |
| | | | off) | | | | |
| 45 | Normal | ″ | Excellent | ″ | ″ | ″ | ″ |
| | (Smooth, | | (No cracks | | | | |
| | No “skin”) | | or flaking | | | | |
| | | | off) | | | | |
| 60 | Normal | Slight | Excellent | ″ | ″ | ″ | ″ |
| | (Smooth, | | (No cracks | | | | |
| | No “skin”) | | or flaking | | | | |
| | | | off) | | | | |
| 225 | Normal | Yes | Excellent | ″ | ″ | ″ | ″ |
| | (Smooth, | | (No cracks | | | | |
| | No “skin”) | | or flaking | | | | |
| | | | off) | | | | |
| 1200 | Normal | Curling | Excellent | ″ | n/d | n/d | Mottled & |
| | (Smooth, | up on | (No cracks | | | | lumpy |
| | No “skin”) | itself | or flaking | | | | |
| | | | off) | | | | |

TABLE 4
Integrity and Handling Characteristics of Trilayer Dressings

| Exposure | | | | | | | |
|---|---|---|---|---|---|---|---|
| Time | | | | | During Hydration | | |
| to 100% | | | | | | Force | |
| Humidity | Prior To Hydration | | | | | Required | After |
| @ 37° C. | Surface | | | | Speed of | for | Hydration |
| (minutes) | Appearance | Curling | Integrity | Flexibility | Hydration | Hydration | Appearance |
| 0 | Normal | No | Good; some | No | Normal | No | Normal |
| | | | delamination | | | | |
| 15 | Irregular | No | Good; some | Yes | Slow | No | Mottled |
| | | | delamination | | | | |
| 60 | Skinned | Yes | Good; some | Yes | Very Slow | Yes | Very |
| | | | delamination | | | | Mottled |
| | | | | | | | and lumpy |

Conclusions:

The monolithic dressings were fully functional at very high levels of residual moisture. As much as 28% moisture was found to retain complete functionality. When the moisture levels rose to 44%, the dressings were still functional, however some of their activity was reduced Higher levels of moisture may also retain some function. The original dressings, at 2.5% moisture content, were not flexible, but had all the other desired properties including appearance, a flat surface, integrity, rapid and uncomplicated hydration and a smooth appearance post hydration. Once the moisture content was increased to 5.8%, the monolithic dressings became flexible, while retaining their functionality and desirable characteristics. They retained their flexibility, without curling or losing their integrity or appearing to form excessive amounts of fibrin prior to hydration.

This contrasted with the tri-layer dressings, which began to lose their desirable characteristics upon the addition of moisture, and lost them entirely by the time moisture had increased to 33%. At no time did these dressings become flexible.

### Example 3

For dressings utilizing a backing, the backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes. For dressings without backing material, PETG 2.4×2.4 cm molds were placed in a −80° C. freezer for at least 60 minutes.

For all dressings, ERL fibrinogen lot 3114 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. Once prepared the thrombin was placed on ice until use. The temperature of the fibrinogen and thrombin prior to dispensing was 4° C.±2° C. Molds were removed from the −80° C. freezer and placed on a copper plate that was placed on top of dry ice. A repeat pipettor was filled with fibrinogen and second repeat pipettor was filled with thrombin. Simultaneously 2 ml of fibrinogen and 300 micro liters of thrombin were dispensed into each mold. Once the molds were filled they were returned to the −80° C. freezer for at least two hours before being placed into the freeze dryer. Dressings were then lyophilized as described below.

Both groups were performance tested in the EVPA assay. In addition, the group which had a backing was also tested in the Adherence and Weight Held assays.

Results:

| | EVPA # | | | Weight | Weight |
|---|---|---|---|---|---|
| | Pass/ | Peel Test | Adherence | Held | Held |
| Group | Total | Adherence | Std Dev | (mean) (g) | Std Dev |
| Backing | 6/6 | 3.7 | 0.5 | 153 | 37.3 |
| No Backing | 9/12 | | | | |

Conclusions:

Dressings formulated with backing material performed well, with all dressings passing the EVPA test, and high values for adherence and weight held. Dressings without backing material were not quite as effective in the EVPA assay, however, surprisingly 75% of them passed the EVPA test. Without the backing the other tests could not be performed. The ability of the dressings made without a backing to succeed in the EVPA assay indicates that these dressings would be effective in treating arterial injuries and even more effective in treating venous and small vessel injuries.

### Example 4

For all dressings, ERL fibrinogen lot 3130 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. For the group with shredded VICRYL™ mesh dispersed within, this support material was cut into approximately 1 mm×1 mm pieces and dispersed within the thrombin solution prior to filling the molds. Once prepared the thrombin was placed on ice until use. The temperature of the fibrinogen and thrombin prior to dispensing was 4° C.±2° C. Cylindrical molds made of 10 or 3 mL polypropylene syringes (Becton Dickinson) with the luer-lock end removed were used. The plungers were withdrawn to the 6 mL and 2 mL mark respectively. For dressings utilizing a backing, the support material was cut and placed into each mold and pushed down until it was adjacent to the plunger. Once prepared the molds were placed upright and surrounded by dry ice, leaving the opening exposed at the top. 1 ml of fibrinogen and 0.15 mL of thrombin (with or without backing material dispersed within) were dispensed into the 10 mL molds and 1 ml of fibrinogen and 0.15 mL of thrombin (with or without support material dispersed within) were dispensed into the 3 mL molds, which were allowed to freeze for 5 minutes. The molds were then placed into the −80° C. freezer for at least two hours before being placed into the freeze dryer and lyophilized as described above.

Upon removal from the lyophilizer, both groups were performance tested in a modified EVPA assay. Briefly, a plastic foam form was slipped over the artery. This covering had a hole in it that corresponded to the hole in the artery and the surrounding tissue. Warm saline was added to the surface of the dressing and the mold was immediately passed down thru the hole in the foam to the artery surface. The plunger was then depressed and held by hand for 3 minutes, after which the mold was withdrawn as the plunger was depressed further. At this point the artery was pressurized and the assay continued as before.

Results

| | | EVPA Result | Maximum | |
|---|---|---|---|---|
| Support Material | Mold Size | (@250 mmHg) | Pressure | |
| None | 10 ml | Pass | >250 | mmHg |
| Dexon Mesh Backing | 10 ml | Pass | ″ | |
| ″ | 3 ml | Pass | ″ | |
| Shredded Dexon | 10 ml | Pass | ″ | |
| Mesh (Dispersed) | | | | |
| ″ | 3 ml | Fail | 150 | mm Hg |

Conclusions:

Dressings that included no backing or a DEXON™ mesh backing performed well, with all passing the EVPA test at 250 mmHg. When the support material was dispersed throughout the composition, the dressings also performed well, with the large size (10 mL mold) dressings holding the full 250 mmHg of pressure, while the smaller held up to 150 mmHg of pressure. This indicates that the use of a support material may be optional, and it's location may be on the ‘back’ of the dressing, or dispersed thou the composition, as desired.

### Example 5

Dressings made with a support material on the “back” (i.e. the non wound-facing side) of the dressing were manufactured by first cutting the mesh support material and placing it into each PETG 10×10 cm mold. Twenty-five microliters of 2% sucrose was pipetted on top of each of the four corners of the backing material. Once completed the molds were placed in a −80° C. freezer for at least 60 minutes.

For dressings made with a support material on the “front” (i.e. the wound-facing side) of the dressing, these were manufactured without any support material in the mold. The support mesh was placed atop the dressing immediately after dispensing of the fibrinogen and thrombin into the mold (see below), and lightly pressing it into the surface prior to its freezing. In all other ways the manufacture of the dressings was similar as described below.

For all dressings, ERL fibrinogen lot 3114 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. Once prepared the thrombin was placed on ice until use. The thrombin was adjusted to deliver 0.1 units/mg of Fibrinogen or 25 Units/ml thrombin. Once prepared the thrombin was placed on ice until use. The temperature of the fibrinogen and thrombin prior to dispensing was 4° C.±2° C. The mold was removed from the −80° C. freezer and placed on an aluminum plate that was placed on top of dry ice. The aluminum plate had a 0.25 inch hole drilled in the center and a fitting attached so that a piece of tubing could be attached to a vacuum source. The mold was centered over the hole in the aluminum plate and vacuum was turned on. The vacuum served two purposes it prevented the mold from moving and it held it flat against the aluminum plate. Thirty-five milliliters of fibrinogen and 5.25 milliliters of Thrombin were placed in 50 ml test tube, inverted three times and poured into the mold. Once the molds were filled and the support material applied as described above, they were returned to the −80° C. freezer for at least two hours before being placed into the freeze dryer. Dressings were then lyophilized as described previously.

Both groups were performance tested in the EVPA assay. In addition, the group which had a backing was also tested in the Adherence and Weight Held assays.

Results:

| Support Material | EVPA # | | | Weight | Weight |
|---|---|---|---|---|---|
| (Mesh) | Pass/ | Adherence | Adherence | Held | Held |
| Orientation | Total | Test Score | Std Dev | (mean) (g) | Std Dev |
| Back (away from | 6/6 | 3.5 | 0.5 | 136 | 49 |
| injury site) | | | | | |
| Front | 6/6 | 3.8 | 0.4 | 163 | 32 |
| (immediately | | | | | |
| adjacent to | | | | | |
| injury site) | | | | | |

Conclusions:

Dressings formulated with backing material in either orientation well, with all dressings passing the EVPA test, and high values for adherence and weight held. This indicates that the location of a support material may be on the ‘back’ of the dressing, or the ‘front’, of the composition as desired.

### Example 6

Backing material (DEXON™) was placed into 2.4×2.4 cm PETG molds. Twenty-five microliters of 2% sucrose was pipetted on top of each of the four corners of the backing material. Once completed the molds were placed in a −80° C. freezer for at least 60 minutes.

Fibrinogen (Enzyme Research Laboratories™ (ERL) lot 3114) was formulated in CFB. The fibrinogen concentration was adjusted to 37.5 mg/ml using CFB. The final pH of the fibrinogen was 7.4±0.1. Once prepared the fibrinogen was placed on ice until use.

Thrombin was formulated in CTB. The final pH of the thrombin was 7.4±0.1. The thrombin concentrations were adjusted with CFB to produce 12.5 units/mg of Fibrinogen (upon mixing), which corresponded to 3120 Units/ml thrombin prior to mixing. Once prepared the thrombin was placed on ice until use.

The temperature of the fibrinogen and thrombin prior to dispensing was 4° C.±2° C. Molds were removed from the −80° C. freezer and placed on a copper plate that was precooled on top of dry ice. A repeat pipettor was filled with fibrinogen and second repeat pipettor was filled with thrombin. Two ml of fibrinogen and 300 micro liters of thrombin were dispensed simultaneously into each mold. Once the molds were filled they were returned to the −80° C. freezer for at least two hours before being placed into the freeze dryer. They were then lyophilized as described below, and performance tested using the EVPA and Adherence Assays as described below.

### Example 7

Backing material was placed into each 1.5×1.5 cm PVC molds. Fifteen microliters of 2% sucrose was pipetted on top of each of the four corners of the backing material. A second piece of PETG plastic was fitted on top of the 1.5×1.5 molds and held in place. This formed a closed mold. The molds were then placed in a −80° C. freezer for at least 60 minutes. Fibrinogen (ERL lot 3100) was formulated in CFB. The fibrinogen concentration was adjusted to 37.5 mg/ml using CFB. The final pH of the fibrinogen was 7.4±0.1. Once prepared the fibrinogen was placed on ice until use. Thrombin was formulated in CTB. The final pH of the thrombin was 7.4±0.1. The thrombin concentrations were adjusted using CTB to deliver the following amounts 2.5, 0.25, 0.1, 0.05, 0.025, 0.016, 0.0125 and 0.01 units/mg of Fibrinogen (upon mixing), which corresponded to 624, 62.4, 25, 12.5, 6.24, 3.99, 3.12, and 2.5 Units/ml thrombin prior to mixing. Once prepared the thrombin was placed on ice until use. The temperature of the fibrinogen and thrombin prior to dispensing was 4° C.±2° C. Molds were then removed from the −80° C. freezer and placed on an aluminum plate that was pre-cooled on top of dry ice. Three holes were punched at the top of the mold using an 18 gauge needle. One hole was used for injecting fibrinogen, the second for injecting thrombin, and the third hole served as a vent to release air that was displaced from inside the mold. A pipette was then filled with fibrinogen and a second pipette with thrombin. Simultaneously 0.78 ml of fibrinogen and 0.17 ml of thrombin were injected via these pipettes into each mold. Once filled the molds were placed on top of a pool of liquid nitrogen for thirty seconds and then returned to the −80° C. freezer for at least two hours before being placed into the freeze dryer. They were then lyophilized as described below, and performance tested using the EVPA and Adherence Assays as described below.

### Example 8

Backing material was placed into 2.4×2.4 cm PVC molds. Twenty-five microliters of 2% sucrose was pipetted on top of each of the four corners of the backing material. Once completed the molds were placed in a −80° C. freezer for at least 60 minutes. Fibrinogen (ERL lot 3100) was formulated in CFB. The fibrinogen concentration was adjusted to 37.5 mg/ml using CFB. The final pH of the fibrinogen was 7.4±0.1. Once prepared the fibrinogen was placed on ice until use. Thrombin was formulated in CTB. The final pH of the thrombin was 7.4±0.1. Using CTB, the thrombin concentrations were adjusted to deliver the following amounts 0.125, 0.025, 0.0125, 0.00625 and 0.0031 units/mg of Fibrinogen upon mixing, which corresponded to 31.2, 6.24, 3.12, 1.56 and 0.78 Units/ml thrombin prior to mixing. Once prepared the thrombin was placed on ice until use. The temperature of the fibrinogen and thrombin prior to dispensing was 4° C.±2° C. The molds were removed from the −80° C. freezer and placed on an aluminum plate that that was precooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Example 9

Backing material was placed into PVC 2.4×2.4 cm molds. Twenty-five microliters of 2% sucrose was pipetted on top of each of the four corners of the backing material. Once completed the molds were placed in a −80° C. freezer for at least 60 minutes. A vial containing 3 grams of Fibrinogen (Sigma™ Lot# F-3879) was removed the −20° C. freezer and placed at 4° C. for 18 hours. The bottle was then removed from the freezer and allowed to come to room temperature for 60 minutes. To the bottle, 60 ml of 37° C. water was added and allowed to mix for 15 minutes at 37° C. Once in solution the fibrinogen was dialyzed against incomplete fibrinogen buffer (IFB, which was CFB without HSA and Tween™) for 4 hours at room temperature. At the end of the four hours HSA was added to a concentration of 80 mg/g of total protein, and Tween™ 80 (animal source) was added to a concentration of 15 mg/g total protein. The final pH of the fibrinogen was 7.4±0.1. The fibrinogen concentration was then adjusted to 37.5 mg/m with CFB. Once prepared the fibrinogen was placed on ice until use. Thrombin was formulated in CTB. The final pH of the thrombin was 7.4±0.1. Using CTB, the thrombin concentrations were adjusted to deliver the following amounts 2.5, 0.25, 0.125, 0.083 and 0.0625 units/mg of Fibrinogen (upon mixing) which corresponded to 624, 62.4, 31.2, 20.8 and 15.6 Units/ml thrombin prior to mixing. Once prepared the thrombin was placed on ice until use. The temperature of the fibrinogen and thrombin prior to dispensing was 4° C.±2° C. Molds were removed from the −80° C. freezer and placed on an aluminum plate that was that was precooled on top of dry ice. A 3 ml syringe fitted with an 18 gauge needle was filled with 2 ml of fibrinogen and a second 1 ml syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Example 10

Backing material was placed into 2.4×2.4 cm PVC molds. Twenty-five microliters of 2% sucrose was pipetted on top of each of the four corners of the backing material. A second piece of PETG plastic was cut to fit on top of the molds and held in place by clips located at each end of the mold, producing closed molds. Once completed the molds were placed in a −80° C. freezer for at least 60 minutes. Fibrinogen (ERL lot 3060 was formulated in CFB. The final pH of the fibrinogen was 7.4±0.1. The fibrinogen concentration was adjusted to 37.5 mg/ml using CFB. Once prepared the fibrinogen was placed on ice until use. Thrombin was formulated in CTB. The final pH of the thrombin was 7.4±0.1. Using CTB, thrombin concentrations were adjusted to deliver the following amounts 2.5, 0.25, 0.125, 0.083 and 0.062 units/mg of Fibrinogen (after mixing), which corresponded to 624, 62.4, 31.2, 20.8, and 15.6 Units/ml thrombin (prior to mixing). Once prepared the thrombin was placed on ice until use. The temperature of the fibrinogen and thrombin prior to dispensing was 4° C.±2° C. Molds were removed from the −80° C. freezer and placed on an aluminum plate that was that was precooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Example 11

Backing material was placed into 2.4×2.4 cm PVC molds. Twenty-five microliters of 2% sucrose was pipetted on top of each of the four corners of the backing material. A second piece of PETG plastic was cut to fit on top of the 2.4×2.4 molds and held in place by the use of clips located at each end of the mold to create closed molds. The molds were then placed in a −80° C. freezer for at least 60 minutes. A vial containing 3 grams of Fibrinogen (Sigma Lot# F-3879) was removed the −20° C. freezer and placed at 4° C. for 18 hours. The bottle was then removed from the freezer and allowed to come to room temperature for 60 minutes. To the bottle, 60 ml of 37° C. water was added and allowed to mix for 15 minutes at 37° C. Once in solution the fibrinogen was dialyzed against IFB. At the end of the four hours HSA was added to a concentration of 80 mg/g of total protein, and Tween™ 80 (animal source) was added to a concentration of 15 mg/g total protein. The final pH of the fibrinogen was 7.4±0.1. The fibrinogen concentration was adjusted to 37.5 mg/ml using CFB. Once prepared the fibrinogen was placed on ice until use. Thrombin was formulated in CTB. The final pH of the thrombin was 7.4±0.1. Thrombin concentration was adjusted to deliver the following amounts 2.5, 0.25, 0.125, 0.1 and 0.083 units/mg of Fibrinogen (upon mixing), which corresponded to 624, 62.4, 31.2, 24.96 and 20.79 Units/ml thrombin (before mixing). Once prepared the thrombin was placed on ice until use. The temperature of the fibrinogen and thrombin prior to dispensing was 4° C.±2° C. Molds were removed from the −80° C. freezer and placed on an aluminum plate that was that was pre-cooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

### Example 12

Backing material was placed into 2.4×2.4 cm PVC molds. Twenty-five microliters of 2% sucrose was pipetted on top of each of the four corners of the backing material. A second piece of PETG plastic was cut to fit on top of the molds and held in place by the use of clips located at each end of the mold to create closed molds. Once completed, the molds were placed in a −80° C. freezer for at least 60 minutes.

A vial containing 3 grams of Fibrinogen (Sigma™ Lot# F-3879) was removed from the −20° C. freezer and placed at 4° C. for 18 hours. The bottle was then allowed to come to room temperature for 60 minutes. To the bottle, 60 ml of 37° C. water was added and allowed to mix for 20 minutes at 37° C. Once in solution, the fibrinogen was dialyzed against IFB. At the end of the four hours, human serum albumin (HSA) was added to a concentration of 80 mg/g of total protein, and Tween™ 80 (animal source) was added to a concentration of 15 mg/g total protein. The final pH of the fibrinogen was 7.4±0.1. The fibrinogen concentration was adjusted to 37.5 mg/ml using CFB. Once prepared the fibrinogen was placed on ice until use.

Thrombin was formulated in CTB. The final pH of the thrombin was 7.4±0.1. Thrombin was adjusted to deliver the following amounts 2.5, 0.25, 0.125, 0.08 and 0.06 units/mg of Fibrinogen (after mixing), which corresponded to 624, 62.4, 31.2, 20.8 and 15.6 Units/ml thrombin (prior to mixing). Once prepared the thrombin was placed on ice until use. The temperature of the fibrinogen and thrombin prior to dispensing was 4° C.±2° C. Molds were removed from the −80° C. freezer and placed on an aluminum plate that was that was precooled on top of dry ice. A 3 ml syringe fitted with an 18 gauge needle was filled with 2 ml of fibrinogen and a second, 1 ml, syringe fitted with a 22 gauge needle was filled with 0.3 ml of thrombin. The contents of both syringes were dispensed simultaneously into each mold. Once filled the molds were placed on top of liquid nitrogen for thirty seconds and then returned to the −80° C. freezer for at least two hours before being placed into the freeze dryer. They were then lyophilized as described below, and performance tested using the EVPA and Adherence Assays as described below.

Trilayer (Sandwich) Dressings

Trilayer dressings were produced using the process described in U.S. Pat. No. 6,762,336, using the same sources of fibrinogen and thrombin as utilized to produce the monolithic dressings above.

Results

The results of the EVPA and Adherence Assays are shown in FIGS. 1A and 1B, respectively.

Conclusions:

Dressings produced with between 2.5 to 0.025 thrombin Units/mg of fibrinogen were active in both assays, while those with greater or lesser ratios of thrombin to fibrinogen were not. Significantly greater activity was seen over the range of 2.5 to 0.05 thrombin Units/mg of fibrinogen. Greatly improved performance was seen between the ranges of 0.25 to 0.062 thrombin Units/mg of fibrinogen, while optimum performance was seen between the ranges of 0.125 to 0.08 thrombin Units/mg of fibrinogen. This contrasted with the dressings produced using the process described in U.S. Pat. No. 6,762,336 which reached full performance at 12.5 thrombin Units/mg of fibrinogen, with unacceptable performance occurring as the thrombin concentration was diminished below 12.5 thrombin Units/mg of fibrinogen, with essentially no activity remaining at 1.4 thrombin Units/mg of fibrinogen. This difference in both the limits of performance and the optimum levels is all the more profound given that the performance of the trilayer dressings from U.S. Pat. No. 6,762,336 was decreased by the use of decreasing amounts of thrombin, while the dressing described herein showed an increased activity over this range.

### Example 13

Backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes. Enzyme Research Laboratories (ERL) fibrinogen lot 3114 was formulated in CFB. In addition, HSA was added to 80 mg/g of total protein and Tween 80 (animal source) was added to 15 mg/g total protein. The final pH of the fibrinogen was 7.4+/−0.1. The fibrinogen concentration was adjusted to 37.5 mg/ml. Once prepared the fibrinogen was placed on ice until use. Thrombin was formulated in 150 mM Sodium Chloride, 40 mM Calcium Chloride, 10 mM Tris and 100 mM L-Lysine with the addition of Human Serum Albumin at 100 ug/ml. The final pH of the thrombin was 7.4+/−0.1. The thrombin was adjusted to deliver 0.1 units/mg of fibrinogen or 25 Units/ml thrombin. Once prepared the thrombin was placed on ice until use. The temperature of the fibrinogen and thrombin prior to dispensing was 4° C.+/−2° C. Molds were removed from the −80 C freezer and placed on an aluminum plate that was placed on top of dry ice. A repeat pipettor was filled with fibrinogen and second repeat pipettor was filled with thrombin. Simultaneously 2 ml of fibrinogen and 300 micro liters of thrombin were dispensed into each mold. Once the molds were filled they were returned to the −80° C. freezer for at least two hours before being placed into the freeze dryer. One group of dressings was lyophilized on day 0, while the remainders were kept frozen at −80° C. A second group of dressings were lyophilized on day seven and a third group was lyophilized on day fourteen.

Once all the dressings had been lyophilized, they were tested using the EVPA, Adherence, and Weight Assays described herein.

Results:

| Days | | | | | |
|---|---|---|---|---|---|
| Frozen | | | | | |
| Prior to | EVPA # | | | Weight | Weight |
| Freeze | Pass/ | Peel Test | Adherence | Held | Held |
| Drying | Total | Adherence | Std Dev | (mean) (g) | Std Dev |
| 0 | 5/6 | 3.5 | 0.5 | 168.0 | 63.2 |
| 7 | 6/6 | 3.8 | 0.4 | 164.7 | 29.4 |
| 14 | 6/6 | 3.7 | 0.5 | 139.7 | 39.7 |

Conclusions:

The compositions of fully mixed, frozen fibrinogen and thrombin remained stable and functional for 7 and 14 days, with no apparent degradation in their performance. Longer storage would be expected to produce similar results.

### Example 14

Backing material was cut and placed into each PETG 2.4×2.4 cm mold. Twenty-five microliters of 2% sucrose was pipetted on top of each of the four corners of the backing material. Once completed the molds were placed in a −80° C. freezer for at least 60 minutes.

Dressings Group 1 (no Albumin, no Tween 80): Enzyme Research Laboratories (ERL) Fibrinogen lot 3130 was formulated in 100 mM Sodium Chloride, 1.1 mM Calcium Chloride, 10 mM Tris, 10 mM Sodium Citrate, and 1.5% Sucrose. The final pH of the fibrinogen was 7.4+/−0.1. The fibrinogen concentration was adjusted to 37.5 mg/ml.

Dressings Group 2 (no Albumin, Tween 80): ERL Fibrinogen was formulated in 100 mM Sodium Chloride, 1.1 mM Calcium Chloride, 10 mM Tris, 10 mM Sodium Citrate, and 1.5% Sucrose. Tween 80 (animal source) was added to 15 mg/g total protein. The final pH of the fibrinogen was 7.4+/−0.1. The fibrinogen concentration was adjusted to 37.5 mg/ml.

Dressings Group 3 (Albumin, no Tween 80): ERL Fibrinogen was formulated in 100 mM Sodium Chloride, 1.1 mM Calcium Chloride, 10 mM Tris, 10 mM Sodium Citrate, and 1.5% Sucrose. HSA was added to 80 mg/g of total protein. The final pH of the fibrinogen was 7.4+/−0.1. The fibrinogen concentration was adjusted to 37.5 mg/ml.

Dressings group 4 (Albumin, Tween 80): ERL Fibrinogen was formulated in 100 mM Sodium Chloride, 1.1 mM Calcium Chloride, 10 mM Tris, 10 mM Sodium Citrate, and 1.5% Sucrose (Fibrinogen complete buffer). In addition, HSA was added to 80 mg/g of total protein and Tween 80 (animal source) was added to 15 mg/g total protein. The final pH of the fibrinogen was 7.4+/−0.1. The fibrinogen concentration was adjusted to 37.5 mg/ml.

Once prepared, the fibrinogen solutions were placed on ice until use.

Thrombin was formulated in 150 mM Sodium Chloride, 40 mM Calcium Chloride, 10 mM Tris and 100 mM L-Lysine with the addition of HSA at 100 ug/ml. The final pH of the thrombin was 7.4+/−0.1. The thrombin was adjusted to deliver 0.1 Units/mg of fibrinogen or 25 Units/ml thrombin.

Once prepared the thrombin solution was placed on ice until use.

The temperature of the fibrinogen and thrombin solutions prior to dispensing was 4° C.+/−2° C. Molds were removed from the −80° C. freezer and placed on an aluminum plate that was placed on top of dry ice. A repeat pipettor was filled with fibrinogen solution and second repeat pipettor was filled with thrombin solution. Simultaneously 2 ml of fibrinogen solution and 300 micro liters of thrombin solution were dispensed into each mold. Once the molds were filled they were returned to the −80° C. freezer for at least two hours before being placed into the freeze dryer.

Results:

| | EVPA # | | | Weight | Weight |
|---|---|---|---|---|---|
| | Pass/ | | Adherence | Held | Held |
| Formulation | Total | Adherence | Std Dev | (mean) (g) | Std Dev |
| −Alb − Tween | 0/6 | 0.8 | 1.0 | 24.0 | 26.3 |
| −Alb + Tween | 3/6 | 3.3 | 0.8 | 114.7 | 40.8 |
| +Alb − Tween | 1/6 | 1.7 | 1.0 | 45.0 | 39.9 |
| +Alb + Tween | 5/6 | 3.5 | 0.5 | 131.3 | 32.0 |

Conclusions:

The results show that the addition of Albumin improved dressing performance. The addition of Tween improved performance even further. The combination of both resulted in the best performance.

### Example 15

Molds consisted of a pair of aluminum plates, separated by a plastic spacer of 3/16″ Plexiglas with 1″×1″ square notches cut into it. In use, the open side of the notches was oriented to the top of the vertically mounted plate-spacer-plate “sandwich” which together formed the mold for the dressings. The mold was pre-cooled by submersion in dry ice pellets, with the top standing slightly above the dry ice. Backing material was then cut and placed into each mold. ERL fibrinogen was formulated in CFB. The final pH of the fibrinogen was 7.4±0.1. The fibrinogen concentration was adjusted to produce dressings with 13 mg/cm² of fibrinogen in the final product. Thrombin was formulated in CTB. The final pH of the thrombin was 7.4±0.1. The thrombin was adjusted to deliver 0.1 Units/mg of fibrinogen in the final dressing.

Fibrinogen and thrombin were chilled to the required temperatures (2, 4, 6 and 8° C.) and mixed in a pre-chilled 15 mL conical tube and mixed using a vortex at high speed for 5 seconds, prior to dispensing into the molds. The fibrinogen-thrombin mixture was then pipetted into the molds using a serological pipette.

Results:

| | | | Biochemical Characterization | |
|---|---|---|---|---|
| | Performance Testing | | % Aα | |
| Initial Temperature | EVPA | | Converted | % of γ Chain |
| of Fibrinogen and | (Passed/ | Adherence | to Free α | Converted to |
| Thrombin (° C.) | Tested) | (Mean) | Chain | γ-γ Dimer |
| 2 | 5/5 | 4.0 | 51 | 0 |
| 4 | 5/5 | 4.0 | 38 | 0 |
| 6 | 5/5 | 3.8 | 57 | 0 |
| 8 | 4/4 | 4.0 | 44 | 0 |

### Example 16

The purpose of this study was to analyze the STB dressing (described in U.S. patent application #2008/0031934 A1) according to the method for determining fibrin content described in Stroetmann U.S. Pat. No. 4,442,655.

However, there are some complexities that arise with this course of action. As described in the Stroetmann patent, the thrombin and fibrinogen mixed within the preparation of the invention are to be intentionally reacted together to produce an amount of fibrin of at least ‘10% by weight’ in the final product. In addition, the patent teaches that a preparation with ‘less than 10% by weight’ of fibrin is ineffective when it states that “in the case of fibrin content of less than 10% by weight, the fibrinogen nature of the dry preparation is prevailing so that the material is brittle and has an insufficient mechanical strength”. Among them are discrepancies in terminology and unclear method descriptions, which given that the Stroetmann patent was filed almost 30 years ago and was translated from German, are not unexpected. However, this does create difficulties in understanding the older techniques described within the patent and applying them to analysis of modern products.

One major issue is the difference between the definition of Stroetmann fibrin used by Stroetmann and the modern definition of fibrin that would be understood by someone of ordinary skill in the art. In the Stroetmann patent the term fibrin is never explicitly defined, however the patent text clearly indicates that the term fibrin is used to describe fibrin that is soluble in an aqueous solution. This is demonstrated in the patent's method description for the “Determination of the fibrin content” shown above, in which it is apparent that the dry preparation described therein was easily dissolved upon hydration with 0.9% NaCl solution. The solution was then “separated from the insoluble constituents” so that the resulting supernatant, which would contain only soluble proteins, could be run on a gel and analyzed for fibrin content. No mention was made of a way to determine if any other type of fibrin was present in these “insoluble constituents” and since only soluble protein can penetrate into a gel, it would not be possible to analyze insoluble protein using Stroetmann's gel analysis method.

Additionally, Stroetmann teaches that fibrin that is soluble in solution is important to the invention when he describes the desired fibrin as “a soluble fibrin polymer predominantly cross-linked in a longitudinal direction” and states that “the particularly preferred fibrin polymer is fibrin predominantly cross-linked in longitudinal direction”. Thus, it is obvious that Stroetmann was most interested in fibrin that was capable of being solubilized in an aqueous solution and that this soluble fibrin was the only kind being measured by his assay. From this assay, the proportions of fibrin and fibrinogen were then supposed to be determined from the gel, although no way to identify fibrin on the gel was specified, however given that fibrin is a polymer of fibrinogen, it would be reasonable to assume that Stroetmann considered any bands of a molecular weight larger than fibrinogen (330 kDa) to be fibrin.

However, there remains an even greater issue with the ‘655’ Patent, namely that it is in fact impossible to generate a value for “% by weight” based solely upon photometric data as suggested in the ‘665’ Patent. Photometric data taken off of gel electrophoresis is no more than semi-quantitative at best, and cannot be used to measure absolute mass as it is only an estimate of relative quantities of proteins. However, as is known to one of ordinary skill in the art, a quantitative mass measurement may be done by measuring the mass (i.e. ‘weight’) of material in the test sample (i.e. the subject of the Invention), and doing the same at each processing step of the analysis in order to determine the mass of material subjected to the photometric measurements. This is necessary since during processing prior to the photometric measurement, portions of the original material may be removed, (such as when ‘insoluble material’ is removed per the ‘655’ Patent) which would render the final calculation of a % by weight impossible.

It should be noted that the term “% by weight” is archaic and while the use of the term ‘weight’ in place of ‘mass’ may be tolerated¹ the overall term “% by weight” is no longer encouraged as it is an improper term for what is formally described as the ‘Mass Fraction’ or ‘Mass Percent’³, i.e. the proportion of a mixture's mass which is composed of the component material being measured.

Therefore, in order to determine the relative amount of ‘fibrin’ in the material in question, the proportion of the original mass of material retained in the sample to be subjected to photometric analysis must be measured. Only then, in combination with the strictly proportional estimation of certain bands on the gel via the photometric analysis, can the ‘% by weight’ of the constituents of the original sample (i.e. the ‘Invention’) be determined.

Material

4″×4″ STB Dressings from Lot#091809 and Lot#092509

50 ml Conical Tubes

10 ml Serological Pipettes

0.9% NaCl Solution

15 ml Conical Tubes

Plungers from 25 ml Eppendorf Combitips

Siliconized Microcentrifuge Tubes

BCA Protein Assay Kit (Thermo Scientific/Pierce cat#23225/23227)

Non-Denaturing NativePAGE™ Gel System (Invitrogen NativePAGE™ Novex® 4-16% Bis-Tris Gel System)

Non-Reducing SDS-PAGE Gel System (Invitrogen 4% Novex® Tris-Glycine Gel System) *Copies of the BCA Assay kit protocol and the gel system instruction manuals have been included as Attachments 1-3 at the end of this report.

Methods

I. Standard Functional Performance Testing

A. Each 4″×4″ STB dressing was cut into 16 sections of approximately 1″×1″ each and every other section was tested (for a total of 8 sections from each 4″×4″ dressing) in the standard functional performance assays: the EVPA Assay and the Adherence Assay.
B. The EVPA Assay was performed according to STB SOP# P-500 Ex Vivo Porcine Arteriotomy Assay (EVPA), version 1.0. (see Attachment 4)
C. The Adherence Assay was performed according to STB SOP# P-501 Adherence Assay for Dressings Made with Backing Material, version 1.0. (see Attachment 5)

II. Sample Preparation for the Determination of Fibrin Content According to the Method from Stroetmann U.S. Pat. No. 4,442,655 (For further clarification, a flow chart summarizing the sample preparation steps is also displayed in FIG. 1.)

D. Carefully removed the mesh backing from the four 4″×4″ STB dressings.
E. Weighed each dressing and recorded the masses.
F. Transferred all of the dressings to a grinder and ground them together for 5 seconds in order to form a powder.
G. Weighed out 1 gram portions of the powderized dressings into each of eight 50 ml conical tubes (Tubes #1-8).
H. Warmed 100 ml of the 0.9% NaCl solution to 37° C. and kept the rest of the solution at 24° C. (room temperature).
I. Prepared the 8 dressing samples according to four different methods. Each method was performed at two different temperatures, 24° C. and 37° C. according to the following chart:
J. Centrifuged supernatant samples to remove any insoluble pieces of the clotted dressing.
1. Transferred the supernatant samples to siliconized microcentrifuge tubes.
2. Centrifuged the supernatant samples for 5 minutes at 10,000 rpm and collected the supernatants.

II. Total Protein Assay Measured by BCA Protein Assay Kit

K. Prepared the standards for the standard curve by diluting the stock albumin solution in water as described in the kit protocol (see Attachment 1).
L. Diluted the centrifuged supernatant samples 1:2 and 1:4 in water (Also assayed undiluted supernatant samples).
M. Prepared the BCA Assay Reagent Mixture as described in the kit protocol.
N. Added the standards and samples to 96-well plates. Added 25 ul of the appropriate standard or sample per well.
O. Added 200 ul of the BCA Assay Reagent Mixture to each well.
P. Sealed the plates and incubated them for 30 minutes at 37° C.
Q. Measured the absorbance of all the samples in a plate reader set at 562 nm.
R. Generated a standard curve and used it to calculate the total protein concentrations of the samples, as described in the kit protocol.

III. Non-Denaturing NativePAGE™ Analysis

S. Using the total protein concentration determined for each sample in the BCA Protein Assay, the supernatant samples were all diluted with the appropriate Sample Buffer and ddH₂O so that ˜5 μg of protein could be loaded into each lane of the gel.
T. Ran gels according to the manufacturer's Instruction Manual (see Attachment 2) until the dye front reached the bottom of the gel.
U. Fixed the proteins in the gel by heating them in a microwave for 45 seconds in a solution of acetic acid, methanol, and ddH₂O as per the Instruction Manual.
V. Placed gels in Colloidal Blue stain overnight to stain the protein bands.
W. Removed the dye absorbed by the gel but not bound to the proteins by destaining them in ddH₂O until the background, non-bound stain was removed.
X. Scanned the gels to obtain an image for further analysis and then placed the gels in a Gel Drying Solution to prepare the gels for drying.
Y. Placed the gels in a drying rack between 2 sheets of nitrocellulose wetted with the Gel Drying Solution. Allowed the gels to air dry.

IV. Non-Reducing SDS-PAGE Analysis

Z. Using the total protein concentration determined for each sample in the BCA Protein Assay, the supernatant samples were all diluted with the appropriate Sample Buffer and ddH₂O so that ˜5 μg of protein could be loaded into each lane of the gel.
AA. Ran gels according to the manufacturer's Instruction Manual (see Attachment 3) until the dye front reached the bottom of the gel.
BB. Placed gels in Colloidal Blue stain for 3-12 hrs. to stain the protein bands.
CC. Removed the dye absorbed by the gel but not bound to the proteins by destaining them in ddH₂O until the background, non-bound stain was removed.
DD. Scanned the gels to obtain an image for further analysis and then placed the gels in a Gel Drying Solution to prepare the gels for drying.
EE. Placed the gels in a drying rack between 2 sheets of nitrocellulose wetted with the Gel Drying Solution. Allowed the gels to air dry.

V. Densitometry Analysis of Gels

FF. Used Image J Software to determine the peak area for each visible band in the gel. (ImageJ Software is an image analysis software from the NIH commonly used for densitometry analysis of SDS-PAGE gels).
GG. Opened the scanned gel file in the Image J file, and converted the color image to 32-bit gray scale for better resolution and visualization of the bands.
HH. Shadowed the gel image from the North to facilitate clear identification of the peaks from the background.
II. From left to right, selected each lane to be analyzed in the gel by outlining a rectangular section in the leftmost lane and copying the same section to each lane of interest.
JJ. Generated a cross-section of each rectangular area, revealing the peaks present in each lane of the gel. The darker the band in the gel, the larger the peak in the cross-section.
KK. Delineated each peak from the background by drawing a line at the beginning and the end of the peak and along the bottom.
LL. Determined the area encompassed by each peak by the using the wand tool from the program and selecting the peak for analysis.
MM. After all of the peak areas were determined, designated all of the peaks above the molecular weight of the highest fibrinogen peak (˜330 kDa) as “fibrin” for the purpose of this analysis.
NN. Designated the peaks of fibrinogen and those falling below the molecular weight of fibrinogen as “not fibrin” for the purpose of this analysis.
OO. Calculated the total peak area (“fibrin”+“not fibrin”) for each sample.
PP. Calculated the proportion of “fibrin” in each sample as follows:

peak area of “fibrin” in the sample÷total peak area of the sample

QQ. Calculated the percentage of “fibrin” by weight in each sample as follows:

proportion of “fibrin” in each sample x % protein by weight in each sample

Results

5.1 Standard Functional Performance Testing

In order to determine that the STB dressing lots to be used in these studies exhibited good performance in the standard functional assays normally used to evaluate them, a 4″×4″ STB dressing from Lot#091809 and one from Lot#092509 were tested for functional performance using the EVPA and Adherence Assays. The averaged results from the assays are presented below in Table 5:

TABLE 5
EVPA and Adherence Assay Results

| result | Lot 091809 | Lot 092509 |
|---|---|---|
| % Positive/Pass | 100 | 100 |
| % Pass Adherence | 100 | 100 |
| Adherence Score | 4 out of 5 | 4 out of 5 |

According to their results, the 4″×4″ STB dressings from both lots perform well in the standard functional performance assays.

5.2 Sample Preparation for the Determination of Fibrin Content According to the Method from Stroetmann U.S. Pat. No. 4,442,655

Two 4″×4″ STB dressings from Lot#091809 and two 4″×4″ STB dressings from Lot#092509 were used for the sample preparation. Their weights prior to being powderized are listed below in Table 6.

TABLE 6
Weights of 4″ × 4″ STB Dressings
Used for First Sample Preparation:

| Lot # | Dressing # | Total Mass w/o Mesh |
|---|---|---|
| 091809 | 52 | 3.12 g |
| 091809 | 136 | 3.25 g |
| 092509 | 8 | 3.20 g |
| 092509 | 127 | 3.35 g |

Sample Appearance after Initial Incubation for 5 Minutes or 30 Minutes (Tubes #1-8):

All dressing samples in tubes #1-8 were hydrated with 10 ml of 0.9% NaCl solution, vortexed briefly, and then immediately incubated for either 5 minutes or 30 minutes, at 24° C. or 37° C. A range of conditions were investigated since Stroetmann did not specify the conditions used in his method. After this initial incubation step, the samples appeared to have clotted completely and had formed large, white clots of insoluble fibrin under all conditions tested. No supernatant was visibly detectable for any of the samples.

Sample Appearance after Clot Compression Following Initial Incubation (Tubes #1-4):

Since no supernatant could be collected after the initial incubation step, the clots in tubes #1-4 were firmly compressed to force out any liquid confined within the clot itself. All clots formed during the initial incubation were white, dense, and very difficult to compress. Consequently, it was only possible to extrude and collect small volumes of supernatant (between 300-550 ul per sample) for analysis.

Sample Appearance after Second Incubation for 5 Minutes or 30 Minutes (Tubes #5-8):

After the initial incubation step and a further addition of 10 ml of 0.9% NaCl solution, tubes #5-8 were incubated again at the same temperature for either 5 minutes or 30 minutes more to determine if the additional solution could extract soluble components from the clot. After this second incubation step, a small amount of the NaCl solution had been absorbed into the clot, while the rest remained sitting on top of the clot. This volume of supernatant (approximately 9 to 9.5 ml per sample) was then collected for analysis.

Sample Appearance after Clot Compression Following Second Incubation (Tubes #5-8):

Once the supernatant was decanted after the second incubation step and saved for analysis, the clots in tubes #5-8 were compressed to expel any additional liquid remaining within the clot. All clots were still intact and appeared to be as white, dense, and difficult to compress as those from tubes #1-4. Volumes of supernatant ranging between 0.8-1.85 ml per sample were extruded and collected for analysis.

5.3 Total Protein Assay

Descriptions of the supernatant samples collected throughout the sample preparation are listed in Table 7, along with the sample codes used to more concisely identify them.

TABLE 7
Supernatant Sample Codes and Descriptions

| Sample | |
|---|---|
| Code | Supernatant Sample Description |
| 1 - AC | Tube #1 - After Compression: Initial Incubation for 5 min. at |
| | 24° C. & Compress Clot |
| 2 - AC | Tube #2 - After Compression: Initial Incubation for 5 min. at |
| | 37° C. & Compress Clot |
| 5 - BC | Tube #5 - Before Compression: Second Incubation for 5 min. at |
| | 24° C. |
| 5 - AC | Tube #5 - After Compression: Second Incubation for 5 min. at |
| | 24° C. & Compress Clot |
| 6 - BC | Tube #6 - Before Compression: Second Incubation for 5 minutes |
| | at 37° C. |
| 6 - AC | Tube #6 - After Compression: Second Incubation for 5 min. at |
| | 37° C. & Compress Clot |
| 3 - AC | Tube #3 - After Compression: Initial Incubation for 30 min. at |
| | 24° C. & Compress Clot |
| 4 - AC | Tube #4 - After Compression: Initial Incubation for 30 min. at |
| | 37° C. & Compress Clot |
| 7 - BC | Tube #7 - Before Compression: Second Incubation for 30 min. at |
| | 24° C. |
| 7 - AC | Tube #7 - After Compression: Second Incubation for 30 min. at |
| | 24° C. & Compress Clot |
| 8 - BC | Tube #8 - Before Compression: Second Incubation for 30 min. at |
| | 37° C. |
| 8 - AC | Tube #8 - After Compression: Second Incubation for 30 min. at |
| | 37° C. & Compress Clot |

All of these supernatant samples were evaluated at multiple dilutions in the Total Protein Assay. The total protein concentration for each dilution and the total amount of protein present in each supernatant sample were determined and are presented in Table 8.

TABLE 8
Total Protein Results for Supernatant Samples

| | | | | | Total | Total |
|---|---|---|---|---|---|---|
| | | | | Average | Supernatant | Protein in |
| | Protein in | Protein in 1:2 | Protein in 1:4 | of Protein | Volume | Supernatant |
| Sample | Undiluted | Dilution | Dilution | Concentrations | Collected | Sample |
| Code | (mg/ml) | (mg/ml) | (mg/ml) | (mg/ml) | (ml) | (mg) |
| 1-AC | 0.611 | 0.564 | 0.645 | 0.607 | 0.55 | 0.33 |
| 2-AC | 0.693 | 0.740 | | 0.717 | 0.30 | 0.21 |
| 5-BC | 0.217 | 0.210 | 0.201 | 0.209 | 9.00 | 1.88 |
| 5-AC | 0.704 | 1.149 | 1.485 | 1.113 | 1.85 | 2.06 |
| 6-BC | 0.183 | 0.192 | 0.163 | 0.179 | 9.50 | 1.70 |
| 6-AC | 0.806 | 1.320 | 1.721 | 1.282 | 1.50 | 1.92 |
| 3-AC | 0.644 | 0.614 | 0.842 | 0.700 | 0.45 | 0.32 |
| 4-AC | 0.708 | 1.175 | 0.658 | 0.847 | 0.45 | 0.38 |
| 7-BC | 0.201 | 0.245 | 0.182 | 0.209 | 9.00 | 1.88 |
| 7-AC | 0.706 | 1.208 | 1.438 | 1.117 | 0.80 | 0.89 |
| 8-BC | 0.313 | 0.317 | 0.343 | 0.324 | 9.00 | 2.92 |
| 8-AC | 0.916 | 1.336 | 1.690 | 1.314 | 1.50 | 1.97 |

These results indicate that only small amounts of protein (˜3 mg or less) were detectable in any of the supernatant samples.

A 4″×4″ STB dressing consists of numerous components besides the active ingredients, fibrinogen and thrombin. The dry weight and percentage of total weight for each of the main components* used to formulate a standard 4″×4″ dressing were calculated and are listed in Table 9. The dry weight of each component in a 1 gram sample of powderized dressing was also calculated and is shown in Table 9 as well.

TABLE 9
Weights of STB Dressing Components
Weight of Fibrinogen and Fibrinogen Buffer
Components in an STB Dressing

| | Weight of | % of Total Dry | In 1.0 g | |
|---|---|---|---|---|
| | Component | Component | of an STB | TOTAL |
| | in 4″ × 4″ | Weight | Dressing, | Weight |
| | Dressing | in 4″ × 4″ | there are | of all |
| Component | (mg) | Dressing | (mg): | Components |
| Fibrinogen, | 1302.00 | 49.3 | 481.46 | |
| Clottable | | | | |
| NaCl | 245.45 | 9.3 | 90.76 | |
| Na Citrate | 123.52 | 4.7 | 45.68 | |
| CaCl₂ | 5.13 | 0.2 | 1.90 | |
| Tris | 50.88 | 1.9 | 18.81 | |
| Sucrose | 630.00 | 23.9 | 232.97 | |
| Tween 80 | 19.53 | 0.7 | 7.22 | |
| Albumin | 102.58 | 3.9 | 37.93 | |
| Component | 2479.09 | 93.9 | 916.74 | 916.74 |
| Weight | | | | |
| SUBTOTAL | | | | |

Weight of Thrombin Buffer Components in an STB Dressing

| | Weight of | % of Total Dry | In 1.0 g | |
|---|---|---|---|---|
| | Component | Component | of an STB | |
| | in 4″ × 4″ | Weight | Dressing, | |
| | Dressing | in 4″ × 4″ | there are | |
| Component | (mg) | Dressing | (mg): | TOTAL |
| NaCl | 57.86 | 2.2 | 21.39 | |
| CaCl₂ | 29.31 | 1.1 | 10.84 | |
| Tris | 7.99 | 0.3 | 2.96 | |
| l-Lysine | 66.00 | 2.5 | 24.41 | |
| Component | 161.16 | 6.1 | 59.60 | 976.34 |
| Weight | | | | |
| SUBTOTAL | | | | |

Weight of Water in an STB Dressing

| | Weight of | % of Total Dry | In 1.0 g | |
|---|---|---|---|---|
| | Component | Component | of an STB | |
| | in 4″ × 4″ | Weight | Dressing, | |
| | Dressing | in 4″ × 4″ | there are | |
| Component | (mg) | Dressing | (mg): | TOTAL |
| Water | 64.00 | 2 | 23.67 | |
| Component | | | 23.67 | 1000.0 |
| Weight | | | | |
| SUBTOTAL | | | | |

* Note that only the approximate weights of components added to the dressing formulation by STB were calculated; any additional proteins and/or buffer salts already present in the formulation components are not known and thus were not included. However, based on the calculations above, the amounts of these are minimal.

These calculations show that much of the weight of an STB dressing is due to components other than protein and that the majority of the protein weight is from fibrinogen and albumin, which account for approximately 481.5 mg and 37.9 mg respectively, of a 1 gram sample of powderized dressing. Using these numbers as the starting weight of protein in each 1 gram dressing sample, the percentage of this protein present in the supernatant samples was calculated (using the ‘total protein in supernatant sample’ values from Table 8 above), thereby expressing the amount of protein in each individual supernatant sample as a percent by weight. The results for the supernatant samples taken from the same tube were also summed together as paired supernatant samples. These results are all displayed in Table 10.

TABLE 10
Total Protein in Supernatant Samples as Percents by Weight

| | | Total Protein | Total Protein | | |
|---|---|---|---|---|---|
| | | in Individual | in Paired | Total Protein | Total Protein |
| | Total Protein | Supernatants | Supernatants | in Individual | in Paired |
| | in Supernatant | as % of | as % of | Supernatants | Supernatants |
| Sample | Sample | Fibrinogen | Fibrinogen | as % of | as % of |
| Code | (mg) | Plus Albumin | Plus Albumin | Fibrinogen | Fibrinogen |
| 1 - AC | 0.33 | 0.064 | 0.069 | 0.069 | 0.069 |
| 2 - AC | 0.21 | 0.041 | 0.044 | 0.045 | 0.045 |
| 5 - BC | 1.88 | 0.363 | 0.759 | 0.391 | 0.819 |
| 5 - AC | 2.06 | 0.396 | | 0.428 | |
| 6 - BC | 1.70 | 0.328 | 0.698 | 0.354 | 0.753 |
| 6 - AC | 1.92 | 0.370 | | 0.399 | |
| 3 - AC | 0.32 | 0.061 | 0.065 | 0.065 | 0.065 |
| 4 - AC | 0.38 | 0.073 | 0.079 | 0.079 | 0.079 |
| 7 - BC | 1.88 | 0.363 | 0.535 | 0.391 | 0.577 |
| 7 - AC | 0.89 | 0.172 | | 0.186 | |
| 8 - BC | 2.92 | 0.562 | 0.941 | 0.606 | 1.016 |
| 8 - AC | 1.97 | 0.379 | | 0.409 | |

Thus, the largest percentage of total protein detected in any of the supernatant samples, individual and paired, was only about 1%, which resulted from the addition of both protein results taken from tube #8. All of the other results were well below 1%. From this data, it appears that the overwhelming majority of the dressing protein is incorporated into the clotted insoluble fibrin and that very little of it can be extracted into any type of supernatant.

5.4 Non-Denaturing NativePAGE™ Analysis

Normally, when analyzing STB dressings in-house by SDS-PAGE, the dressing samples are reduced, by adding β-mercaptoethanol (β-ME) and then heating, prior to being run on a gel. In these gels the SDS (sodium dodecyl sulfate, which is also known as sodium lauryl sulfate) denatures the proteins while the reducing agent breaks the disulfide bonds which hold together the three pairs of polypeptide chains that form the large fibrinogen protein. This enables analysis of the small modifications to these individual polypeptide chains that occur as the fibrinogen is converted into fibrin and then insoluble fibrin.

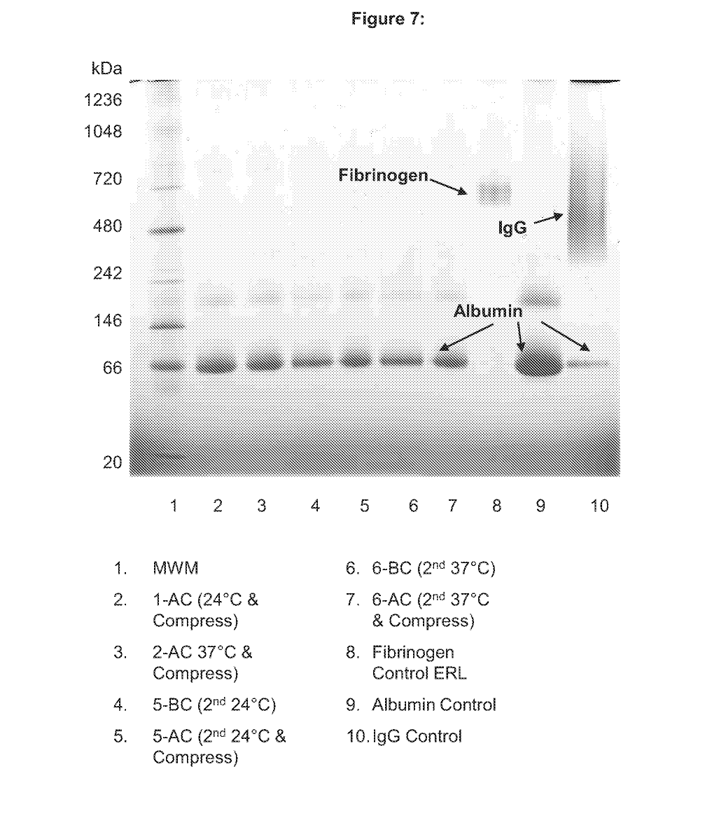

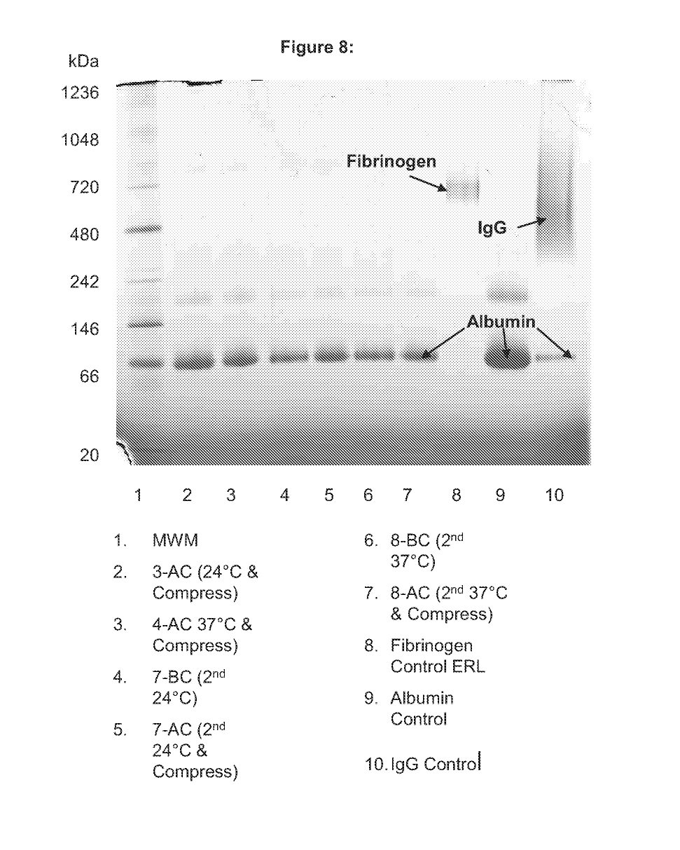

In contrast, the methodology described in the Stroetmann patent instructs that the supernatant be analyzed via gel electrophoresis under non-reducing conditions (without β-ME), which preserves the disulfide bonds within the fibrinogen protein. Because the type of gel system used in the Stroetmann patent was not specified and since keeping the supernatant proteins intact was apparently necessary to determine the difference between fibrinogen and fibrin according to the Stroetmann procedure, we reasoned that evaluation by NativePAGE™ would be the optimal way of accomplishing this. NativePAGE™ is a modern method of protein analysis in which the samples are non-reduced and non-denatured so that the proteins remain intact and in their native conformation. Therefore, following the NativePAGE™ kit procedure the supernatant samples, along with several control samples, were processed under non-reducing conditions using the NativePAGE™ sample buffer and run on NativePAGE™ Novex® 4-16% Bis-Tris gels. The controls tested included unformulated control ERL fibrinogen used to manufacture STB dressings as well as albumin and IgG controls. The resulting gels are displayed in FIGS. 7 and 8.

In both gels shown above, the supernatant samples from all the conditions were fairly comparable. Under the NativePAGE™ gel conditions, the albumin control exhibited two bands, one at the expected molecular weight of 65 kDa and a second band around 200 kDa. Both of these bands were also present in the supernatant samples and thus it appears that most of the protein detected in the supernatants is albumin.

Also, under these conditions the fibrinogen control sample ran as a smeared band at approximately 600-700 kDa, although it was expected to run at the size of the complete fibrinogen protein, ˜330 kDa. In order to verify this discrepancy, fibrinogen samples from multiple sources, along with an IgG sample and albumin samples from several sources were prepared under the three different conditions described below and run on the NativePAGE™ Novex® 4-16% Bis-Tris gels. In addition, an STB dressing control sample was prepared by dissolving a dry piece of unclotted STB dressing in ODS, or Okude Dissolving Solution. ODS is a solution containing urea and SDS that is normally used for the in-house SDS-PAGE analysis.

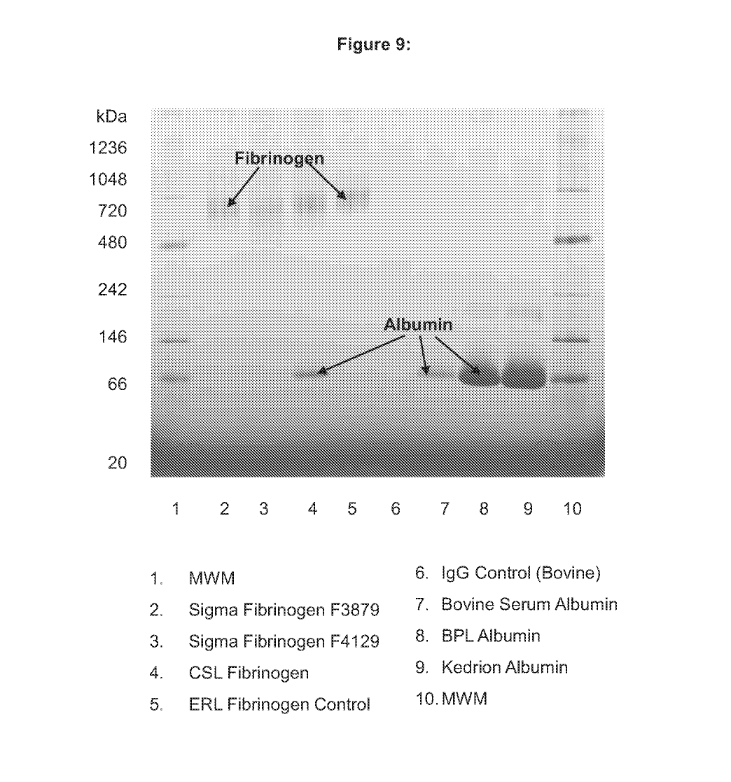

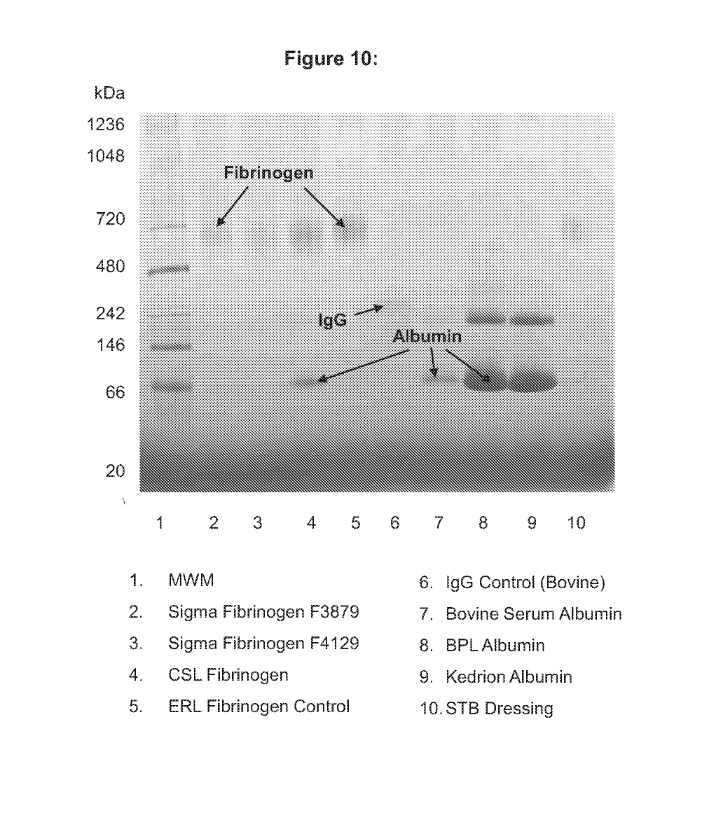

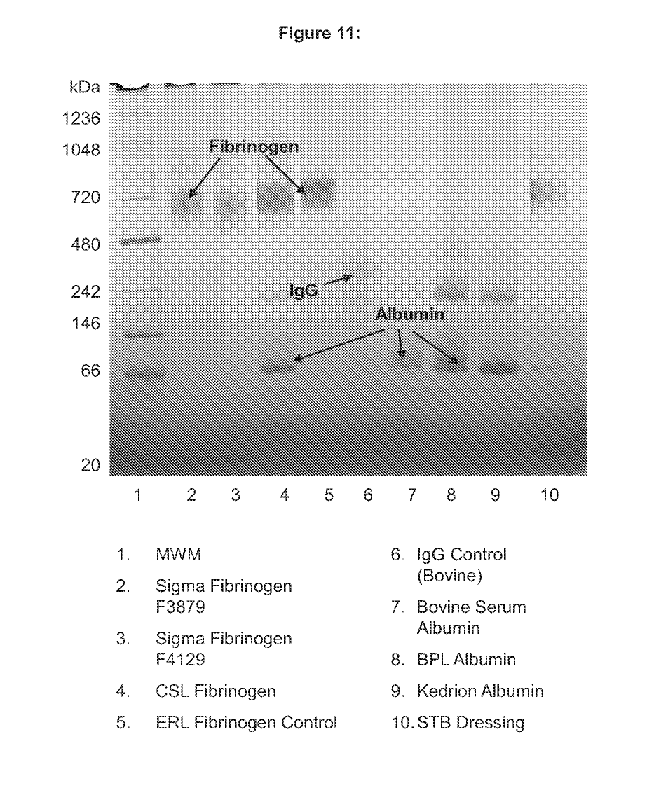

Controls dissolved or diluted in 0.9% NaCl solution according to the NativePAGE™ kit protocol (like those in FIGS. 7 and 8 above) are shown in FIG. 9. Control samples prepared according to the NativePAGE™ kit protocol but using ODS to dissolve the samples are shown in FIG. 10. Additional control samples were also prepared with ODS and then heated for 2 hours at 40° C. prior to being run on the gel and are presented in FIG. 11.

All three sample preparation conditions produced similar results on the NativePAGE™ gels. Once again, the fibrinogen controls appeared as a smeared band around 600-700 kDa with some additional smearing above and below it. The STB dressing control also looked very similar to these controls, which was expected since this dressing sample was not hydrated or activated and thus would contain primarily unpolymerized fibrinogen like the fibrinogen controls. The albumin samples again exhibited two major bands, with a few faint higher molecular weight bands visible when the samples were prepared with ODS. As expected, albumin bands were visible in the CSL fibrinogen and the STB dressing sample since those are the only fibrinogen formulations that contain albumin.

5.5 Non-Reduced SDS-PAGE

Seeing that all of the fibrinogen controls ran at a much higher molecular weight on the NativePAGE™ gels than expected regardless of the sample preparation methods employed, the use of the NativePAGE™ gels was reevaluated. While most proteins form a globular shape in their native conformation, the intact fibrinogen protein forms a more linear structure. Thus its apparent size in relation to other proteins may differ depending on the gel system used and whether it has been denatured or remains in its native conformation.

Since the Stroetmann procedure does not specify the gel system to be used, a literature search was performed to understand more about the SDS-PAGE technologies that existed at the time of the patent filing and that would likely have been familiar to Stroetmann and his contemporaries. One of the most relevant articles found was published in 1970 by McKee⁴ et al. It describes SDS-PAGE analysis of non-reduced and reduced human fibrinogen and fibrin using 5% acrylamide gels and showed a fibrinogen band running at about the expected size of 330 kDa.

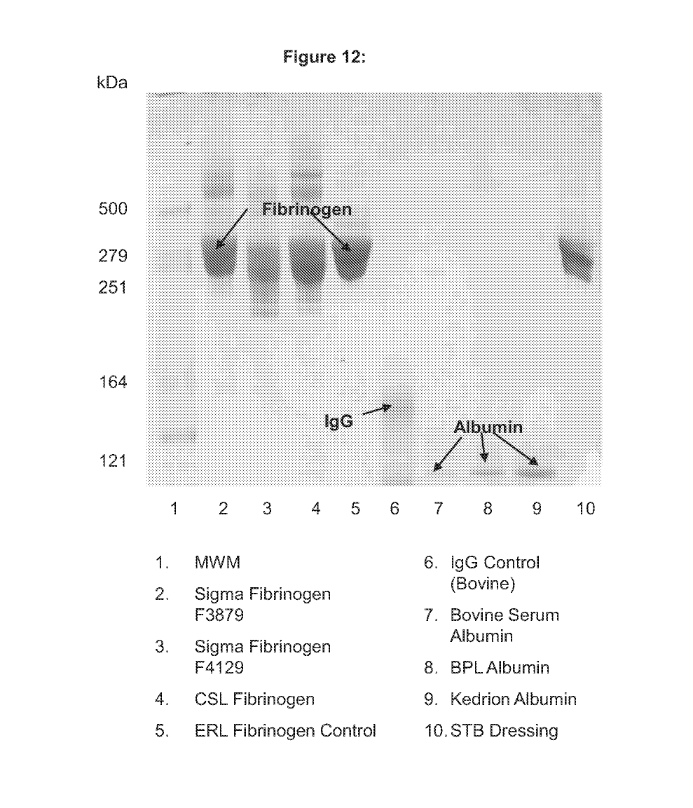

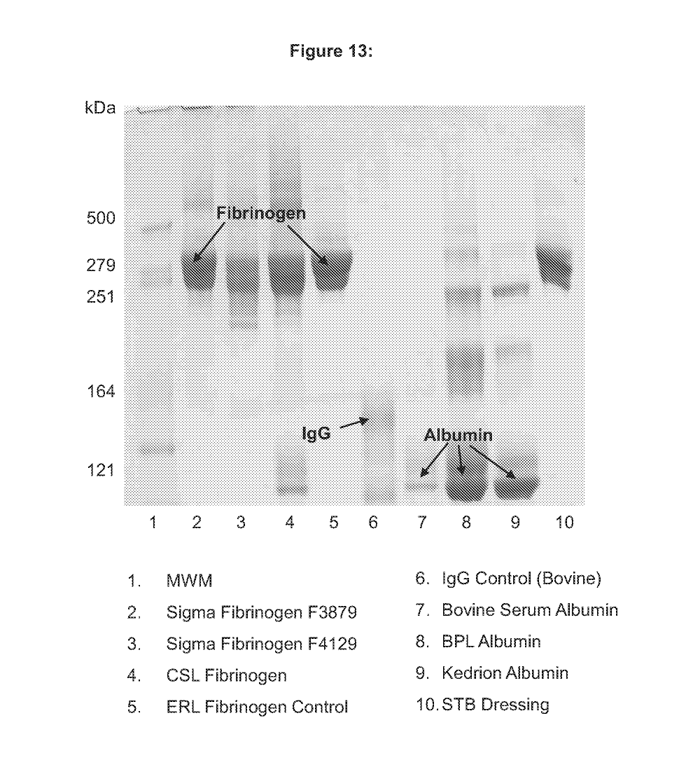

In order to determine if SDS-PAGE analysis on gels similar to those used in this publication would produce comparable results, the controls were analyzed by non-reducing SDS-PAGE only, using 4% Tris-Glycine acrylamide gels. The control samples were prepared with and without ODS and then heated for 2 minutes at 85° C. as instructed in the kit procedure for preparing non-reduced samples. The resulting gels are displayed in FIGS. 12 and 13.

When examined on the 4% Tris-Glycine gels, the non-reduced fibrinogen control samples appeared as two major bands corresponding to the appropriate size of the fibrinogen molecule. The albumin band was still detectable at approximately the right size, as it had been on the NativePAGE™ gels. Consequently, the non-reduced gels appear to be more similar to those likely used by Stroetmann and thus are more appropriate for evaluating the supernatant samples going forward.

5.6 Second Sample Preparation

In order to analyze the experimental supernatants by non-reduced SDS-PAGE, fresh supernatant samples were prepared according to the same procedures used to prepare the first set. Three more 4″×4″ STB dressings from Lots#091809 were used for the second sample preparation. Their weights prior to being powderized are listed below in Table 11.

TABLE 11
Weights of 4″ × 4″ STB Dressings
Used for Second Sample Preparation:

| Lot # | Dressing # | Total Mass w/o Mesh |
|---|---|---|
| 091809 | 99 | 3.23 g |
| 091809 | 120 | 3.13 g |
| 091809 | 56 | 3.06 g |

The clot appearances of the samples were equivalent to those from the first set so no photographs were taken. Once again, no supernatant was detectable following the initial hydration step and only minimal supernatant volumes were able to be recovered upon clot compression.

5.7 Second Total Protein Assay

All of these supernatant samples were next evaluated at multiple dilutions in the Total Protein Assay. The total protein concentration for each dilution and the total amount of protein present in each supernatant sample were determined and are presented in Table 12.

TABLE 12
Total Protein Results for Supernatant Samples

| | | | | | Total | Total |
|---|---|---|---|---|---|---|
| | | | | Average | Supernatant | Protein in |
| | Protein in | Protein in 1:2 | Protein in 1:4 | of Protein | Volume | Supernatant |
| Sample | Undiluted | Dilution | Dilution | Concentrations | Collected | Sample |
| Code | (mg/ml) | (mg/ml) | (mg/ml) | (mg/ml) | (ml) | (mg) |
| 1-AC | 0.629 | 0.961 | 0.656 | 0.749 | 0.55 | 0.41 |
| 2-AC | 0.578 | 0.920 | 0.625 | 0.708 | 0.30 | 0.21 |
| 5-BC | 0.100 | 0.083 | 0.041 | 0.075 | 9.00 | 0.67 |
| 5-AC | 1.488 | 1.440 | 1.098 | 1.342 | 1.85 | 2.48 |
| 6-BC | 0.091 | 0.075 | 0.036 | 0.067 | 9.50 | 0.64 |
| 6-AC | 2.141 | 1.483 | 1.800 | 1.808 | 1.50 | 2.71 |
| 3-AC | 1.020 | 1.523 | 0.704 | 1.082 | 0.45 | 0.49 |
| 4-AC | 1.286 | 1.540 | 0.709 | 1.178 | 0.45 | 0.53 |
| 7-BC | 0.153 | 0.134 | 0.070 | 0.119 | 9.00 | 1.07 |
| 7-AC | 0.819 | 1.568 | 1.085 | 1.157 | 0.80 | 0.93 |
| 8-BC | 0.182 | 0.181 | 0.130 | 0.164 | 9.00 | 1.48 |
| 8-AC | 1.060 | 1.518 | 1.902 | 1.493 | 1.50 | 2.24 |

These results correspond with those determined previously and show that again only small amounts of protein (less than 3 mg) were detectable in any of the supernatant samples.

Using the supernatant total protein values from Table 12, the percentage of total protein that this supernatant protein represented was calculated as a percent by weight based on the total starting weight of protein in each 1 gram dressing sample (of which 481.5 mg is fibrinogen and 37.9 mg is albumin). The resulting percents by weight of protein for each individual supernatant sample and the summations of the paired supernatant samples taken from the same tube are all displayed in Table 13.

TABLE 13
Total Protein in Supernatant Samples as Percents by Weight

| | | Total Protein | Total Protein | | |
|---|---|---|---|---|---|
| | | in Individual | in Paired | Total Protein | Total Protein |
| | Total Protein | Supernatants | Supernatants | in Individual | in Paired |
| | in Supernatant | as % of | as % of | Supernatants | Supernatants |
| Sample | Sample | Fibrinogen | Fibrinogen | as % of | as % of |
| Code | (mg) | Plus Albumin | Plus Albumin | Fibrinogen | Fibrinogen |
| 1 - AC | 0.41 | 0.079 | 0.079 | 0.086 | 0.086 |
| 2 - AC | 0.21 | 0.041 | 0.041 | 0.044 | 0.044 |
| 5 - BC | 0.67 | 0.129 | 0.607 | 0.140 | 0.655 |
| 5 - AC | 2.48 | 0.478 | | 0.516 | |
| 6 - BC | 0.64 | 0.123 | 0.645 | 0.133 | 0.696 |
| 6 - AC | 2.71 | 0.522 | | 0.563 | |
| 3 - AC | 0.49 | 0.094 | 0.094 | 0.101 | 0.101 |
| 4 - AC | 0.53 | 0.102 | 0.102 | 0.110 | 0.110 |
| 7 - BC | 1.07 | 0.206 | 0.384 | 0.222 | 0.415 |
| 7 - AC | 0.93 | 0.178 | | 0.192 | |
| 8 - BC | 1.48 | 0.285 | 0.716 | 0.307 | 0.772 |
| 8 - AC | 2.24 | 0.431 | | 0.465 | |

These results confirm that the amounts of total protein detected in the supernatant samples were all below 1% by weight. Just like the results from the first sample preparation, it appears that almost all of the dressing protein makes up the insoluble fibrin clot and only a small amount can be removed as supernatant.

5.8 Non-Reduced SDS-PAGE

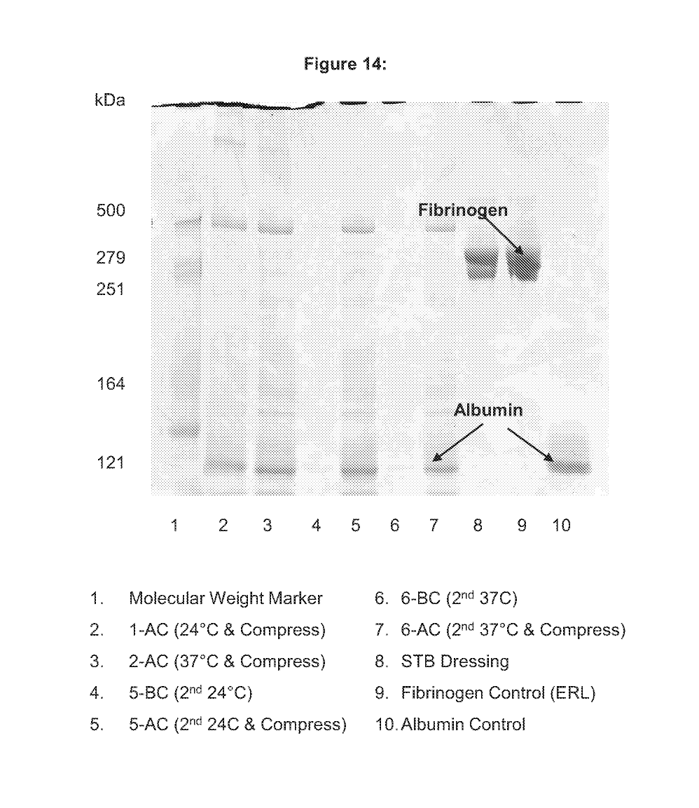

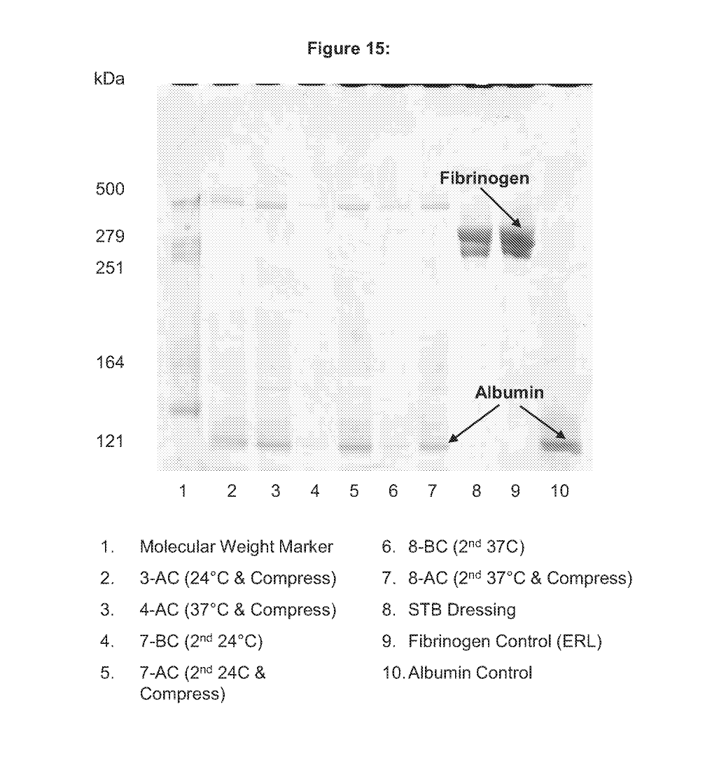

The fresh supernatant samples, along with three control samples, were then analyzed by non-reducing SDS-PAGE. The samples were heated for 2 minutes at 85° C. as instructed in the kit protocol for preparing non-reduced samples and then run on 4% Tris-Glycine gels. The resulting gels are displayed in FIGS. 14 and 15.

The pair of fibrinogen bands that were visible around 240-340 kDa in the unformulated fibrinogen control and the STB dressing control were not detected in the supernatant samples. However the supernatant samples did contain several bands other than fibrinogen. One of these bands is consistent with the albumin control but the identities of the others, including some bands of a higher molecular weight than fibrinogen, are unclear.

According to the method for determining fibrin content outlined in the Stroetmann patent, the band “proportions are photometrically measured and determined after dyeing with Coomassie brilliant blue”. Without additional details about how to perform this measurement, densitometry analysis was chosen for this photometric determination. Densitometry is a standard method for band analysis of SDS-PAGE gels and in this process a scanned image of the SDS-PAGE gel is analyzed with imaging software and the optical densities of the different bands are quantitated.

In order to quantitate the percentage of fibrin visible on the gel, any bands at molecular weights higher than fibrinogen were counted as fibrin. Therefore for densitometry analysis, the areas of the peaks corresponding to all of the bands above fibrinogen (>˜330 kDa) were summed and designated as ‘fibrin’. Additionally, all of the areas of the peaks at or below the peaks of fibrinogen (≦˜330 kDa) were summed and designated as ‘not fibrin’. The ‘fibrin’ results were then divided into the ‘total peak area’ (the summation of the ‘fibrin’ and ‘not fibrin’ results) to get the ‘fibrin proportions determined by densitometry’ (which are presented in the second columns of Tables 14, 15, and 16).

These proportions next needed to be converted into percents by weight, which were used throughout the Stroetmann patent and claims. A method for performing this conversion was not provided, but since there is no way to accurately measure the weight of fibrin already dissolved within a solution, Stroetmann must have calculated the proportion of fibrin in relation to the starting weight of the preparation (1 gram in the Stroetmann method) since that is the only weight data given, and called it percent fibrin by weight. In the case of the Stroetmann preparation in which very little if any insoluble fibrin was present in the preparation, and if we assume that no other protein bands besides fibrinogen were present on the gel, then the proportion of fibrin to fibrinogen seen on the gel would have been roughly equivalent to their percents by weight in the starting preparation.

However, the addition of 0.9% NaCl solution (as required by the Stroetmann method), does not solubilize the STB dressing like it does Stroetmann's preparation. Instead, the 0.9% NaCl solution hydrates and activates the STB dressing to form a large clot of insoluble fibrin. The protein extracted from the clot therefore represents just a fraction of the total protein present in the STB dressing itself and does not include the majority of the protein that exists in the clot as insoluble fibrin. Thus any conversion of the proportion of fibrin to a percent by weight must account for this insoluble fibrin. This enables a direct comparison between the percent fibrin by weight in the STB dressing and the percent fibrin by weight that was measured and claimed in the Stroetmann patent.

To accomplish this, the fibrin proportion determined by densitometry on the gels was multiplied by the percent by weight of total protein in each supernatant sample, which was based on either the 1 gram starting weight of the powderized STB dressing (which is equivalent to Stroetmann's calculations), or according to the more stringent limitation using either the starting fibrinogen and albumin protein weights combined (481.5 mg of fibrinogen plus 37.9 mg of albumin), or the starting fibrinogen protein weight alone (481.5 mg). This gives the final percent fibrin by weight for each individual supernatant sample. The results of these calculations are presented in Tables 9, 10, and 11. The results for the supernatant samples taken from the same tube were also summed together as paired supernatant samples and are shown in Tables 9, 10, and 11 as well.

TABLE 14
Fibrin as % by Weight Based On the Starting 1 gram STB Dressing Weight

| | | | Total Protein | Fibrin as % | Fibrin as % |
|---|---|---|---|---|---|
| | Fibrin | Total Protein | in Individual | by Weight of | by Weight of |
| | Proportions | in Supernatant | Supernatants | 1 gram Dressing | 1 gram Dressing |
| Sample | Determined by | Sample (mg) | as % of 1 | (for Individual | (for Paired |
| Code | Densitometry | (From Table 7) | gram Dressing | Supernatants) | Supernatants) |
| 1 - AC | 0.383 | 0.41 | 0.041 | 0.016 | 0.016 |
| 2 - AC | 0.297 | 0.21 | 0.021 | 0.006 | 0.006 |
| 5 - BC | 0.365 | 0.67 | 0.067 | 0.025 | 0.088 |
| 5 - AC | 0.255 | 2.48 | 0.248 | 0.063 | |
| 6 - BC | 0.425 | 0.64 | 0.064 | 0.027 | 0.034 |
| 6 - AC | 0.024 | 2.71 | 0.271 | 0.007 | |
| 3 - AC | 0.256 | 0.49 | 0.049 | 0.012 | 0.012 |
| 4 - AC | 0.249 | 0.53 | 0.053 | 0.013 | 0.013 |
| 7 - BC | 0.386 | 1.07 | 0.107 | 0.041 | 0.061 |
| 7 - AC | 0.212 | 0.93 | 0.093 | 0.020 | |
| 8 - BC | 0.455 | 1.48 | 0.148 | 0.067 | 0.143 |
| 8 - AC | 0.338 | 2.24 | 0.224 | 0.076 | |

TABLE 15
Fibrin as % by Weight Based On the Starting Fibrinogen & Albumin Weights

| | | Total Protein | Fibrin as % by | Fibrin as % by |
|---|---|---|---|---|
| | | in Individual | Weight of | Weight of |
| | Fibrin | Supernatants as | Fibrinogen Plus | Fibrinogen Plus |
| | Proportions | % by Weight of | Albumin | Albumin |
| Sample | Determined by | Fibrinogen Plus | (for Individual | (for Paired |
| Code | Densitometry | Albumin | Supernatants) | Supernatants) |
| 1 - AC | 0.383 | 0.079 | 0.030 | 0.030 |
| 2 - AC | 0.297 | 0.041 | 0.012 | 0.012 |
| 5 - BC | 0.365 | 0.129 | 0.047 | 0.169 |
| 5 - AC | 0.255 | 0.478 | 0.122 | |
| 6 - BC | 0.425 | 0.123 | 0.052 | 0.065 |
| 6 - AC | 0.024 | 0.522 | 0.013 | |
| 3 - AC | 0.256 | 0.094 | 0.024 | 0.024 |
| 4 - AC | 0.249 | 0.102 | 0.025 | 0.025 |
| 7 - BC | 0.386 | 0.206 | 0.080 | 0.117 |
| 7 - AC | 0.212 | 0.178 | 0.038 | |
| 8 - BC | 0.455 | 0.285 | 0.130 | 0.275 |
| 8 - AC | 0.338 | 0.431 | 0.146 | |

TABLE 16
Fibrin as % by Weight Based On the Starting Fibrinogen Weight Alone

| | | Total Protein | Fibrin as % by | Fibrin as % by |
|---|---|---|---|---|
| | Fibrin | in Individual | Weight of | Weight of |
| | Proportions | Supernatants as | Fibrinogen | Fibrinogen |
| Sample | Determined by | % by Weight of | (for Individual | (for Paired |
| Code | Densitometry | Fibrinogen | Supernatants) | Supernatants) |
| 1 - AC | 0.383 | 0.086 | 0.033 | 0.033 |
| 2 - AC | 0.297 | 0.044 | 0.013 | 0.013 |
| 5 - BC | 0.365 | 0.140 | 0.051 | 0.182 |
| 5 - AC | 0.255 | 0.516 | 0.131 | |
| 6 - BC | 0.425 | 0.133 | 0.056 | 0.070 |
| 6 - AC | 0.024 | 0.563 | 0.014 | |
| 3 - AC | 0.256 | 0.101 | 0.026 | 0.026 |
| 4 - AC | 0.249 | 0.110 | 0.027 | 0.027 |
| 7 - BC | 0.386 | 0.222 | 0.086 | 0.127 |
| 7 - AC | 0.212 | 0.192 | 0.041 | |
| 8 - BC | 0.455 | 0.307 | 0.140 | 0.297 |
| 8 - AC | 0.338 | 0.465 | 0.157 | |

The data in Tables 14-16 show that the percentages of fibrin by weight in the supernatant samples are all less than 0.15% when analyzed using the original weight of the material, which is the conventional method for determining percent by weight that was most likely used by Stroetmann. Even when using other more restrictive methods for determining the percent by weight, such as performing the calculations based only on the protein weights of fibrinogen plus albumin or on the fibrinogen weight alone, the resulting percentages of fibrin by weight are all less than 0.3%.

Discussion

The STB dressing is made utilizing a technology that enables the thrombin and fibrinogen active components to be mixed in such a way as to result in as little conversion of fibrinogen to fibrin or insoluble fibrin as possible. Thus the formation of either (soluble) fibrin or insoluble fibrin is minimized until the STB dressing is hydrated, which activates the components which only then convert to insoluble fibrin.

The STB dressing is a fundamentally different technology than that described in the ‘655’ Patent, where the goal was to produce a material with high levels of (soluble) fibrin in the product, exactly opposite to the goal of the technology of the STB dressing. Unsurprisingly, the results of analyzing the STB dressing were completely opposite to those obtained by Stroetmann in the ‘655’ Patent.

The methods of the ‘665’ Patent do not involve a dressing with a backing material that is not involved in the formation of fibrin per se. Since the calculation of Stroetmann fibrin content in the ‘655’ Patent is on a “% by weight” basis, any component of the dressing that contributes mass without participating in fibrin formation would have the effect of decreasing the “% by weight” of Stroetmann fibrin in the dressing. While it would be correct to include the mass of the backing in this calculation, and thereby decreasing the “% by weight” of Stroetmann fibrin measured in the STB dressing, we chose not to do so in order to shift the analysis in favor of a higher “% by weight of fibrin” in the STB dressing. These higher “% by weight of Stroetmann fibrin” values are approximately 125% larger than those values that would have been calculated had the weight of the mesh been incorporated.

The methods in the ‘655’ Patent are similarly silent with regards to the time and temperature at which the assay for ‘% fibrin by weight’ is to be carried out. This implies that they may be carried out under any reasonable conditions, which would presumably range from ˜4° C. to ˜37° C., with lower temperatures tending to produce less fibrin, and any timeframe from a few seconds to minutes, again with a shorter time presumably resulting in less fibrin formation. In order to attempt to bias in favor of finding fibrin formation, we carried out our studies at both high and low temperatures (24° C. & 37° C.) and for short and long incubation periods (5 and 30 minutes). None of these efforts to increase fibrin formation had any significant effect.

When the dressing was analyzed according to Stroetmann's method for the determination of fibrin content, the STB dressing samples were not solubilized like the preparation of Stroetmann's invention, but instead were activated to form dense clots of insoluble material. This is not surprising given the completely different composition of the materials. No supernatant was visible in any of these clotted samples. Since the analysis described in the ‘655’ Patent assumes a large volume of supernatant to be formed when the dressing is mixed with water, and indeed requires such a supernatant in which to measure the Stroetmann fibrin content of the starting material, the amount of Stroetmann fibrin present in the STB dressing was zero as measured by the method of Stroetmann.

At this point, the analysis according to the method of Stroetmann was complete, however, in order to obtain some supernatant for analysis two methods were employed that went beyond the instructions in the ‘655’ Patent. In one, the clot was firmly compressed to force out any liquid trapped within the clot itself. In the second, a large volume of saline solution was added to the sample to extract any soluble protein from the clot. When analyzed in the total protein assay, these extracted supernatants were found to contain less than 3 mg of protein in the entire supernatant volume, which was only 1% of the weight of fibrinogen in a 1 gram portion of dressing. Therefore, even if all of the protein in all of the excessive supernatant samples turned out to be fibrin, the greatest percent fibrin by weight possible could only be 1%.

When these supernatant samples were analyzed via gel electrophoresis as described by Stroetmann, it was found that the fibrin only comprised a portion of the protein visible on the gel. When this data was combined with the data on the mass of material that had partitioned into the supernatant in order to calculate the actual percent by weight of fibrin, even under the most generous conditions, combining all the materials recovered using the excessive methods described above, the percent fibrin by weight only approached 0.3%, which is well below the 10% fibrin by weight claimed by Stroetmann. A summary of these results is given in Table 17 below.

TABLE 17
Summary of % Fibrin by Weight Data from the Analysis of the
STB Dressing

| | | | | % Fibrin | % |
|---|---|---|---|---|---|
| | | | | by | Fibrin |
| | | | | Weight | by |
| | | | % Fibrin | (As % of | Weight |
| | | | by Weight | 519.4 mg | (As % |
| | | | (As % of 1 | Fibrinogen | of 481.5 |
| | | | gram Total | & | mg |
| Description of | Condition | | Dressing | Albumin | Fibrinogen |
| Variables Tested | Number | Condition Details | Weight) | Weight) | Weight) |
| Initial Condition | 1 | Incubation for 5 min at | 0 | 0 | 0 |
| Approximating | | 24° C. | | | |
| Stroetmann' s | | | | | |
| Method | | | | | |
| Condition # 1 with | 2 | Incubation for 5 min at | 0 | 0 | 0 |
| Increased | | 37° C. | | | |
| Temperature | | | | | |
| Condition #1 with | 3 | Incubation for 30 min | 0 | 0 | 0 |
| Increased | | at 24° C. | | | |
| Incubation Time | | | | | |
| Condition #1 with | 4 | Incubation for 30 min | 0 | 0 | 0 |
| Increased | | at 37° C. | | | |
| Temperature and | | | | | |
| Incubation Time | | | | | |
| Conditions #1-4 | 5 | Incubation for 5 min at | 0.016 | 0.030 | 0.033 |
| with Clot | | 24° C. & Compress | | | |
| Compression | | Clot | | | |
| | 6 | Incubation for 5 min at | 0.006 | 0.012 | 0.013 |
| | | 37° C. & Compress | | | |
| | | Clot | | | |
| | 7 | Incubation for 30 min | 0.012 | 0.024 | 0.026 |
| | | at 24° C. & Compress | | | |
| | 8 | Incubation for 30 min | 0.013 | 0.025 | 0.027 |
| | | at 37° C. & Compress | | | |
| | | Clot | | | |
| Conditions #1-4 | 9 | Initial Incubation for 5 | 0.025 | 0.047 | 0.051 |
| with Additional | | min at 24° C., Add | | | |
| Supernatant Added | | Supernatant & Second | | | |
| and a Second | | Incubation for 5 min at | | | |
| Incubation | | 24° C. | | | |
| | 10 | Initial Incubation for 5 | 0.027 | 0.052 | 0.056 |
| | | at 37° C., Add | | | |
| | | Supernatant & Second | | | |
| | | Incubation for 5 min at | | | |
| | | 37° C. | | | |
| | 11 | Initial Incubation for | 0.041 | 0.080 | 0.086 |
| | | 30 min at 24° C., Add | | | |
| | | Supernatant & Second | | | |
| | | Incubation for 30 min | | | |
| | | at 24° C. | | | |
| | 12 | Initial Incubation for | 0.067 | 0.130 | 0.140 |
| | | 30 min at 37° C., Add | | | |
| | | Supernatant & Second | | | |
| | | Incubation for 30 min | | | |
| | | at 37° C. | | | |
| Conditions #1-4 | 13 | Initial Incubation for 5 | 0.063 | 0.122 | 0.131 |
| with Additional | | min at 24° C., Add | | | |
| Supernatant Added, | | Supernatant & Second | | | |
| Second Incubation, | | Incubation for 5 min at | | | |
| and Clot | | 24° C.; Compress Clot | | | |
| Compression | 14 | Initial Incubation for 5 | 0.007 | 0.013 | 0.014 |
| | | min at 37° C., Add | | | |
| | | Supernatant & Second | | | |
| | | Incubation for 5 min at | | | |
| | | 37° C.; Compress Clot | | | |
| | 15 | Initial Incubation for | 0.020 | 0.038 | 0.041 |
| | | 30 min at 24° C., Add | | | |
| | | Supernatant & Second | | | |
| | | Incubation for 30 min | | | |
| | | at 24° C.; Compress | | | |
| | | Clot | | | |
| | 16 | Initial Incubation for | 0.076 | 0.146 | 0.157 |
| | | 30 min at 37° C., Add | | | |
| | | Supernatant & Second | | | |
| | | Incubation for 30 min | | | |
| | | at 37° C.; Compress | | | |
| | | Clot | | | |
| Summation of | 17 | Initial Incubation for 5 | 0.088 | 0.169 | 0.182 |
| Results from | | min at 24° C., Add | | | |
| Condition #9 + | | Supernatant & Second | | | |
| Condition #13 | | Incubation for 5 min at | | | |
| | | 24° C. | | | |
| Summation of | 18 | Initial Incubation for 5 | 0.034 | 0.065 | 0.070 |
| Results from | | min at 37° C., Add | | | |
| Condition #10 + | | Supernatant & Second | | | |
| Condition #14 | | Incubation for 5 min at | | | |
| | | 37° C. | | | |
| Summation of | 19 | Initial Incubation for | 0.061 | 0.117 | 0.127 |
| Results from | | 30 min at 24° C., Add | | | |
| Condition #11 + | | Supernatant & Second | | | |
| Condition #15 | | Incubation for 30 min | | | |
| | | at 24° C. | | | |
| Summation of | 20 | Initial Incubation for | 0.143 | 0.275 | 0.297 |
| Results from | | 30 min at 37° C., Add | | | |
| Condition #12 + | | Supernatant & Second | | | |
| Condition #16 | | Incubation for 30 min | | | |
| | | at 37° C. | | | |

Conclusion

The analysis of the Stroetmann product for fibrin content following the methodology described in this patent showed a clear difference between the two inventions. In conclusion, the STB dressing was found to contain no measurable fibrin, as determined according to the methods of Stroetmann in the ‘655’ Patent. Even when multiple excessive measures that go well beyond the methods of Stroetmann were employed, the STB dressing was found to contain no more than 0.3% by weight of fibrin.

### Example 17

### Background

To produce an effective hemostatic dressing, STB has developed a process whereby fibrinogen and thrombin can be mixed together without any appreciable conversion of fibrinogen to fibrin, thereby retaining their ability to form an effective, hemostatic dressing.

To determine if the Stroetmann process could likewise produce a hemostatic dressing, dressings will be manufactured using the method of mixing fibrinogen and thrombin described in Stroetmann U.S. Pat. No. 4,442,655 (the ‘655’ Patent). Dressings will be manufactured using STB's standard methodology to provide a basis for comparison and a “hybrid” method combining STB's formulations with Stroetmann's methodology for mixing the fibrinogen and thrombin in order to determine whether the differences observed between the STB process and the resultant product and those of Stroetmann are due solely to differences in composition or processing.

Purpose

The purpose of this study was to manufacture dressings consistent with the methods described in the ‘655’ Patent and then to analyze and compare the biochemical characteristics and functional performance of these dressings to those of STB dressings.

Materials

### 1. Dressing Manufacture

Enzyme Research Laboratories Fibrinogen Lot 3520

STB-prepared Thrombin Lot 1741

Sodium Citrate (Na-Citrate, Sigma)

Calcium Chloride, dihydrate (CaCl₂, Sigma)

STB Fibrinogen Buffer

STB Thrombin Buffer

37° C. Water bath

2-4° C. Ice bath

Dexon™ Mesh #4, 4″×4″ pieces

Dressing Molds

Serological pipettes and dispenser

Pipetmen and tips

Rapid Freezing Tunnel (RFT)

Liquid Nitrogen

Nitrogen gas

Virtis Ultra 35LE Lyophilizer

Argon gas

MarvaSeal bags

AmeriVacS Bag Sealer

Methods

Dressing Manufacture
(a) Standard STB Dressings made according to Example 1.
(b) Dressings Made According to Stroetmann's Methods

The fibrinogen active component will be formulated to a concentration approaching 35 mg/ml in a 20 mM Na-Citrate buffer containing 0.9% NaCl and 25 mM CaCl₂ in the final formulation. (The starting fibrinogen is manufactured in a Na-Citrate buffer and is only available at a maximum concentration of around 40-45 mg/mi.)

The thrombin active component will be added to the fibrinogen at a concentration of 0.06 U thrombin/mg of fibrinogen.

The actives will be maintained at room temperature, mixed, and poured into 4″×4″ polypropylene dressing bases.

The resulting dressings will be incubated at room temperature for 20 minutes and will then be frozen for 10 minutes at −40° C. in the RFTs and lyophilized.
(c) a “Hybrid” Dressings Made According to Stroetmann's Methods but Using the Standard STB Dressing Actives

The fibrinogen and thrombin active components will be formulated in their standard buffers and at their standard concentrations. The fibrinogen will be formulated to 37.5 mg/mL in a solution containing 100 mM NaCl, 1.1 mM CaCl₂*2 H₂O, 10 mM Tris, 10 mM sodium citrate, and 1.5% sucrose. The thrombin will be formulated to 25 IU/mL in a solution containing 150 mM NaCl, 40 mM CaCl₂*2 H₂O, 10 mM Tris, and 100 mM 1-lysine.

The actives will be maintained at room temperature, mixed, and poured into 4″×4″ polypropylene dressing bases.

The resulting dressings will be incubated at room temperature for 5 minutes, 20 minutes, or 12 hours.

The dressings will then be frozen for 10 minutes at −40° C. in the RFTs and lyophilized.

2. Assay Methodology

(a) The moisture content present in a lyophilized dressing was measured by a modified Karl Fischer method. This assay can be used to determine if the lyophilization procedure functioned correctly, as well as to monitor product stability and packaging integrity. Previous experience has established that HDs must have no more than 5% water, with levels below 2.5% being more satisfactory. This assay is performed according to STB SOP# P-503 Moisture Determination Using Brinkman Analyzer, version 1.0.

(b) SDS-PAGE

This assay is used to identify and examine fibrinogen and fibrin proteins in both unclotted and clotted samples. Unclotted human fibrinogen is comprised of two pairs of three polypeptide chains, the alpha (α), beta (β), and gamma (γ) chains. In clotted samples, thrombin along with Factor XIII, catalyzes the conversion of fibrinogen into an opaque cross-linked fibrin clot. As this reaction progresses, small sections of protein are cleaved from both the α and β chains (peptide sections A and B, respectively), and the γ chains cross-link to form γ-γ dimers. The formation of γ-γ dimers is the step whereby fibrinogen is converted to insoluble fibrin, producing the structure that is hemostatic and adherent to injured tissues. The γ-γ dimers band is used to measure the quantity of fibrin present in a sample. When these samples are run on an SDS-PAGE gel, the extent of the changes in the different chains can be visually assessed and if desired, quantitated by densitometry. In this process, a scanned image of the SDS-PAGE gel is analyzed with ImageJ imaging software in order to determine the optical densities of the different fibrinogen chain bands.

The band densities are then compared and the relative amounts of fibrinogen chain modifications are determined. Consequently, the percentage of the Aα chain that has been cleaved to form the smaller α chain and the percentage of γ chain monomer that has been converted to the γ-γ dimer can be calculated. This assay is performed according to STB SOP# P-504 Preparing Fibrinogen-Containing Samples for SDS-PAGE Analysis, Version 1.0 and STB SOP# P-505 Performing SDS-PAGE Analysis on Fibrinogen-Containing Samples, version 1.0.

(c) Ex Vivo Porcine Arteriotomy Assay (EVPA Assay)

This ex vivo assay simulates a puncture wound in a major artery and is the primary assay for evaluating dressing function. In this assay, porcine arteries are mounted over a syringe with a hole drilled into the side. A 2.8 mm diameter hole is made in the artery and a test dressing of approximately 1″×1″ in size is applied to the hole. The syringe is then attached to a controlled pumping system that pressurizes and monitors the pressure within the artery. The artery is pressurized to at least 250 mmHg and the test dressing must maintain this pressure for a period of time, normally 3 minutes, without leaking in order to pass. This assay is performed according to STB SOP# P-500 Ex Vivo Porcine Arteriotomy Assay (EVPA), version 1.0.

Previous experience has shown that the EVPA Assay successfully screens out approximately 90% of fibrin sealant dressings that would go on to fail in vivo. Therefore, the EVPA Assay is an exceptionally useful assay for product evaluation and process development.

(d) Adherence Assay

Earlier studies have also indicated that although the majority of dressings that passed the EVPA Assay functioned extremely well in vivo, there was a subset of dressings that passed the assay but went on to fail in vivo (i.e. gave false positive EVPA results), and that virtually all of these failed due to a lack of adherence to the injured tissue. Accordingly, the Adherence Assay was developed to measure this performance variable ex vivo in order to reduce the overall false positive rate to a negligible value. In the adherence assay, following completion of the EVPA Assay, the syringe with the artery and dressing still in place, is disconnected from the controlled pumping system and is then clamped to a ring stand. A standardized weight in the form of a hemostat is clipped to a top corner of the mesh backing of the test dressing and is allowed to gently peel the dressing from the artery. When the dressing stops peeling, a numerical score from 0 (no adherence) to 4 (>90% adherence) is assigned based on the degree of adherence exhibited. Both the adherence score and the percentage of dressings that receive a passing score (3 or 4) are reported. This assay is performed according to STB SOP# P-501 Adherence Assay for Dressings Made with Backing Material, version 1.0.

Since both the ability to prevent high pressure leaks and to strongly adhere to injured tissue are essential for good dressing performance, the EVPA Assay and the Adherence Assay are routinely paired together to maximize the stringency of the ex vivo testing. When these assays are used in conjunction, dressings are only judged as passing if they pass both the EVPA Assay and have adherence scores of 3 or 4, while those that fail to meet one or more of these criteria are considered failures.

Results

1. Observations from the Manufacture

The following observations were made from the observation of the manufactured dressings. In the preparation of the STB dressing, the fibrinogen and thrombin are mixed at 2-4° C. When fibrinogen and thrombin are mixed at this temperature, they produce a solution that can be poured into the mold without any visual evidence of clotting.

When fibrinogen and thrombin are mixed at room temperature (˜22° C.) according to the formulations and methods described in the Stroetmann Patent, a clot rapidly forms. This is demonstrated when the dressing mold containing the mixture is inverted and cannot be poured or shaken from the mold.

Even when STB's formulations for fibrinogen and thrombin are used in substitution of Stroetmann's formulations at room temperature in the “hybrid” method, a clot rapidly forms that cannot be poured or shaken from the dressing mold.

2. Observations after Lyophilization

(a) After the lyophilization cycle was complete, photographs were taken of each of the dressings produced. The STB dressing was flat and even in texture. The STB dressing was flaky in texture and could be easily cut.

(b) The Stroetmann dressing was also flat and even in texture. However, the Stroetmann dressing was very hard and resembled plaster in consistency and hardness.

(c) The “Hybrid” dressings were very different from both the STB and the Stroetmann Dressings in appearance. These dressings were very uneven in texture and were very thin. They curled in on themselves, with the greatest amount of curl coming from the 12 hr. dressing. They were all hard and difficult to cut or break.

3. Moisture Analysis

The results of the moisture analysis for all of the dressings are presented below in Table 18.

TABLE 18
Moisture Analysis of the Dressing Samples

| | | Standard |
|---|---|---|
| | Average | Deviation |
| Sample | % Moisture | % Moisture |
| STB Dressing | 2.433 | 0.231 |
| Stroetmann Dressing | 2.700 | 0.100 |
| “Hybrid” Dressing, 5 min. | 2.333 | 0.115 |
| “Hybrid” Dressing, 20 min. | 2.167 | 0.153 |
| “Hybrid” Dressing, 12 hrs.. | 2.567 | 0.306 |

Each dressing was assayed in triplicate.

4. Biochemical Assay: SDS-PAGE

After manufacture, two approximately 20 mg samples were taken from each dressing produced. The first 20 mg sample was placed directly into 1.5 mL Okuda Dissolving Solution (ODS) with β-Mercaptoethanol. This is the “unclotted” sample, and is used to determine the amount of fibrinogen to fibrin conversion that occurred during the manufacturing process. The samples were then placed on an orbital rotator for in a 40° C. incubator for a minimum of 1 hr. to allow both the denaturing and reduction of the disulfide linkages in the proteins.

The other 20 mg sample was wetted with 0.2 mL of 0.9% NaCl and placed in a 37° C. incubator for 30 minutes to produce the maximum conversion of fibrinogen to fibrin (the “clotted” sample). After incubation, the reaction was stopped by placing the dressing into ODS. These samples were then placed on an orbital rotator for in a 40° C. incubator for a minimum of 1 hr. to allow both the denaturing and reduction of the disulfide linkages in the proteins.

After preparation, the “unclotted” and “clotted” samples were loaded onto a polyacrylamide gel to which an electrical current is applied. The samples then migrate or “run” down the gel, with the larger proteins remaining near the top and the smaller proteins running to the bottom of the gel. After completion, the gel is then stained and scanned. Densitometry was then performed on the bands: the greater the amount of protein in the band, the darker and denser it appears. This density is quantifiable using Image J computer software.

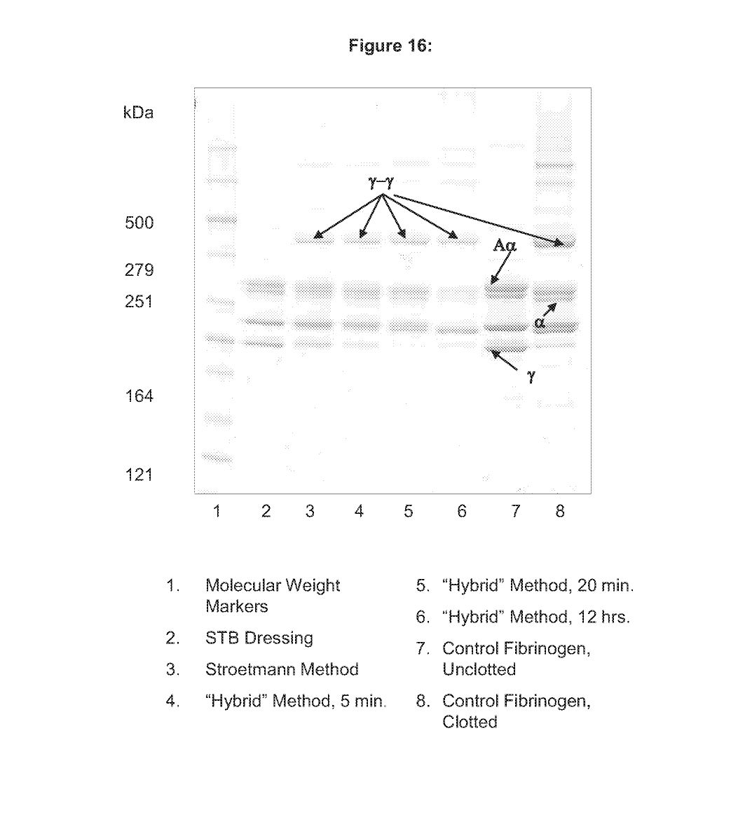

The scanned images of the gels produced with the “unclotted” samples are presented in FIG. 16.

As can be seen in FIG. 16, the dressing made using STB's methodology has no γ-γ dimer band. This indicates that there has been no conversion of the fibrinogen to fibrin in the dressing as it was manufactured.

In the dressings produced using Stroetmann methodology, there has already been significant conversion of the fibrinogen to fibrin in the dressing as it was manufactured. Dressings produced using the “hybrid” Stroetmann process had even higher levels of fibrin formation during production. The densitometry performed on the γ monomer and γ-γ dimer bands in this gel and shown below in Table 19 supports this visual observation.

TABLE 19
Densitometry Results for the “Unclotted” Dressing Samples

| | γ Monomer | γ-γ Dimer | |
|---|---|---|---|
| | Densitometry | Densitometry | % γ-γ |
| Sample | Value | Value | Dimer |
| STB Dressing | 764.1 | 0 | 0 |
| Stroetmann Dressing | 401.1 | 492.4 | 55 |
| “Hybrid” Dressing, 5 min. | 184.6 | 507.1 | 73 |
| “Hybrid” Dressing, 20 min. | 35.5 | 495.1 | 93 |
| “Hybrid” Dressing, 12 hrs.. | 91.8 | 374.7 | 80 |

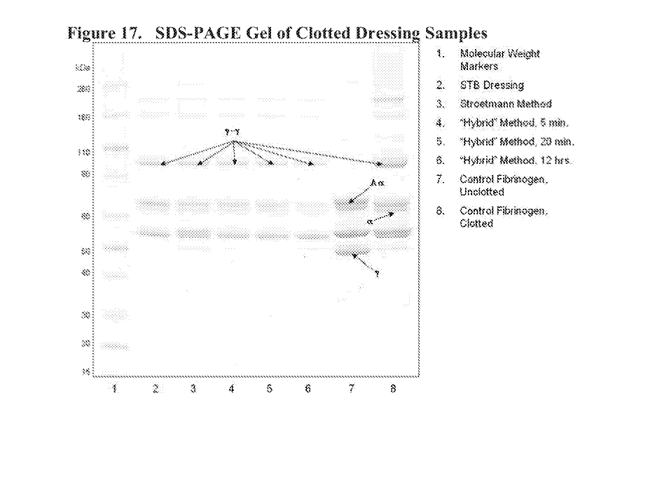

The scanned images of the gels produced with the “clotted” samples are presented in FIG. 17. While having no detectible fibrin formed during production (See FIG. 16 and Table 19 above) it retains the ability to undergo complete fibrin formation as measured by γ-γ dimer band formation (FIG. 17). This indicates that the fibrinogen in the dressing as it was manufactured was capable of undergoing conversion to fibrin to produce a clot.

In all of the dressings, the remaining unclotted fibrinogen in the dressings was converted to fibrin. The densitometry performed on this gel and shown below in Table 20 supports this visual observation.

TABLE 20
Densitometry Results for the “Clotted” Dressing Samples

| | γ Monomer | γ-γ Dimer | |
|---|---|---|---|
| | Densitometry | Densitometry | % γ-γ |
| Sample | Value | Value | Dimer |
| STB Dressing | 0 | 753.4 | 100 |
| Stroetmann Dressing | 0 | 642.1 | 100 |
| “Hybrid” Dressing, 5 min. | 0 | 517.4 | 100 |
| “Hybrid” Dressing, 20 min. | 0 | 513.4 | 100 |
| “Hybrid” Dressing, 12 hrs. | 0 | 481.7 | 100 |

5. Performance Assay: EVPA

After manufacture, each of the 4″×4″ dressings were cut into 16-1″×1″ pieces. Eight of the 16 pieces were evaluated in the EVPA Assay described above. The results of this evaluation are presented below in Table 21.

TABLE 21
EVPA Results for the Dressing Samples

| | | Avg. Pressure ± |
|---|---|---|
| Sample | % Passing | Std. Dev. |
| STB Dressing | 100 | 278 ± 3.5 |
| Stroetmann Dressing | 0 | 27 ± 11.4 |
| “Hybrid” Dressing, 5 min. | 0 | 39 ± 51.0 |
| “Hybrid” Dressing, 20 min. | 0 | 14 ± 10.2 |
| “Hybrid” Dressing, 12 hrs.. | 0 | 8 ± 8 |

As can be seen from Table 21, the dressing produced using STB's methodology had all 8 pieces pass the EVPA Assay, reaching and maintaining pressures of >250 mm Hg. The dressings produced using Stroetmann's methodology and the “Hybrid” method, regardless of the reaction time, did not pass the EVPA Assay, with 0% passing, in both groups and extremely low maximum pressures.

6. Adherence Assay

Immediately after completion of the EVPA Assay, each of the dressing pieces underwent the Adherence Assay. The results of this evaluation are presented below in Table 22.

TABLE 22
Adherence Results for the Dressing Samples

| | | Avg. Pressure ± |
|---|---|---|
| Sample | % Passing | Std. Dev. |
| STB Dressing | 100 | 3.9 ± 0.4 |
| Stroetmann Dressing | 12.5 | 1.4 ± 1.2 |
| “Hybrid” Dressing, 5 min. | 0 | 0.3 ± 0.7 |
| “Hybrid” Dressing, 20 min. | 0 | 0.0 ± 0.0 |
| “Hybrid” Dressing, 12 hrs.. | 0 | 0.0 ± 0.0 |

As can be seen from Table 22, the dressing produced using STB's methodology had all 8 pieces pass the Adherence Assay, with an adherence score of >3. The dressings produced using Stroetmann's methodology and the “hybrid” method did not pass the Adherence Assay, with 12.5% and 0% passing, respectively.

Discussion

The STB dressing is made utilizing a technology that enables the thrombin and fibrinogen active components to be mixed in such a way as to result in as little conversion of fibrinogen to fibrin as possible. Thus the formation of fibrin is minimized until the STB dressing is hydrated on the wound to be treated, which activates the components which only then convert to insoluble fibrin. This is demonstrated in the video of the mixing of the fibrinogen and thrombin, which remains a liquid prior to pouring into the mold and freezing.

The SDS-PAGE gel analysis of the STB dressings supports this visual observation in that no γ-γ dimer band was present in the gel of the “unclotted” (unreacted) dressing samples. However, upon wetting, the fibrinogen and thrombin rapidly converted to a fibrin clot. The SDS-PAGE gel of this clotted sample revealed that there was complete conversion of the fibrinogen to fibrin because of the disappearance of the γ monomer band and appearance of the γ-γ dimer band. In the EVPA and Adherence Assays, the STB dressing adhered to the artery and was able to maintain ≧250 mm Hg for 3 min, indicating full hemostatic functionality of the STB dressings.

The dressings produced using the methodology described in the ‘655’ patent were significantly different from the dressings produced using the STB methodology. Upon mixing in the Stroetmann methodology, the fibrinogen and thrombin reacted rapidly to produce a clot that could not be poured from the dressing mold.

The SDS-PAGE gel analysis of the Stroetmann dressings supports the visual observations in that the γ-γ dimer band was present in the gel of the “unclotted” (unreacted) dressing samples, and that ˜55% of the fibrinogen had converted to fibrin during dressing production. This dressing did not wet easily, and there was no significant increase in the amount of fibrin in the “clotted” sample gel.

In the EVPA and Adherence Assays, the Stroetmann dressing did not adhere to the artery and reached an average pressure of only 27 mm Hg in the EVPA Assay, well below the performance required for hemostasis.

The dressings produced using the “Hybrid” methodology where STB's fibrinogen and thrombin formulations were used with Stroetmann's mixing at room temperature and holding the mixture for up to 12 hrs. were significantly different from the dressings produced using the STB methodology. Upon mixing in this methodology, the fibrinogen and thrombin reacted rapidly to produce a clot that could not be poured from the dressing mold.

The SDS-PAGE gel analysis of the “Hybrid” dressings supports the visual observations in that the dimer band was present in the gel of the “unclotted” (unreacted) dressing samples, and that >70% of the fibrinogen had converted to fibrin. This dressing did not wet easily, and there was no significant increase in the amount of fibrin in the “clotted” sample gel.

In the EVPA and Adherence Assays, the “hybrid” dressing did not adhere to the artery and reached an average pressure of 39 mm Hg in the EVPA Assay.

Conclusions

In conclusion, the STB dressing production process was visibly different from the methods used by Stroetmann in U.S. Pat. No. 4,442,655. The dressings resulting from these processes were visually, chemically, and functionally distinct. Attempts to incorporate elements of the ‘655’ patent process into the STB process resulted in similarly distinct and non-functional dressings.

The lack of function was correlated with the production of fibrin during the process. The incorporation of just a few of the elements of the ‘655’ process into the STB process prevented the production of effective dressings.

Therefore, it is clear that the STB and the ‘655’ processes are distinct, and that the products of these processes are similarly and functionally distinct from each other.

EVPA Performance Testing

Equipment and Supplies:

In-line high pressure transducer (Ashcroft Duralife™ or equivalent)

Peristaltic pump (Pharmacia Biotech™, Model P-1 or equivalent)

Voltmeter (Craftsman™ Professional Model 82324 or equivalent)

Computer equipped with software for recording pressure or voltage information

Tygon™ tubing (assorted sizes) with attachments

Water bath (Baxter Durabath™ or equivalent), preset to 37° C.

Incubation chamber (VWR™, Model 1400G or equivalent), preset to 37° C.

Thermometer to monitor temperatures of both water bath and oven

Assorted forceps, hemostats, and scissors

10 cc. and 20 cc. syringes with an approximately 0.6 cm hole drilled in center and smaller hole drilled through both syringe and plunger. This hole, drilled into the end of the syringe, will be used to keep the plunger drawn back and stationary.

O-rings (size 10 and 13)

Plastic Shields to fit the 10 cc and 20 cc syringes (approximately 3.5 cm in length)

P-1000 Pipetman™ with tips

Sphygmomanometer with neonatal size cuff and bladder

Programmable Logic Controller (PLC) to control the pumps to maintain the desired pressure profile (Optional. Manual control may be used if desired.)

### 1. Materials and Chemicals

Porcine descending aortas (Pel-Freez Biologicals™, Catalog #59402-2 or equivalent)

Cyanoacrylate glue (Vetbond™, 3M or equivalent)

18-gauge needle(s)

0.9% Saline, maintained at 37° C.

Red food coloring

Vascular Punch(es), 2.8 mm or other

Plastic Wrap

### 2. Artery Cleaning and Storage

1. Store arteries at −20° C. until used.
2. Thaw arteries at 37° C. in H₂O bath.
3. Clean fat and connective tissue from exterior surface of artery.
4. Cut the arteries into ˜5 cm segments.
5. The arteries may be refrozen to −20° C. and stored until use.

### 3. Artery Preparation for Assay

1. Turn the artery inside-out so that the smooth, interior wall is facing outwards.
2. Stretch a size 13 O-ring over a 20 cc syringe or a size 10 O-ring over a 10 cc syringe with an approximately 0.6 cm (0.25 in) hole drilled into one side.
3. Pull the artery onto the syringe, taking care not to tear the artery or have a too loose fit. The artery should fit snugly to the syringe. Slide another O-ring of the same size onto the bottom of the syringe
4. Carefully pull both O-rings over the ends of the artery. The distance between the 0-rings should be at least 3.5 cm
5. Using the blade of some surgical scissors, gently scrape the surface of the artery in order to roughen the surface of the artery.
6. Use a 18-gauge needle to poke a hole through the artery over the site of the hole in the syringe barrel (see note above)
7. The tip of the biopsy punch is inserted through the hole in the artery. Depress the punch's plunger to make an open hole in the artery. Repeat a couple of times to ensure that the hole is open and free of connective tissue.
8. Patch holes left by collateral arteries. Generally this is done by cutting a patch from a latex glove and gluing it over the hole with cyanoacrylate glue. Allow the glue to cure for at least 10 minutes.
9. Place the artery in the warmed, moistened container and place in the incubation chamber. Allow the arteries to warm for at least 30 minutes.

### 4. Solution and Equipment Preparation

1. Check to see that the water bath and incubation chamber are maintained at 29-33° C.
2. Make sure that there is sufficient 0.9% saline in the pump's reservoir for completion of the day's assays. Add more if needed.
3. Place 0.9% saline and 0.9% saline with a few drops of red food coloring added into containers in a water bath so that the solutions will be warmed prior to performing the assay.
4. Prepare the container for warming the arteries in the incubation chamber by lining with KimWipes™ and adding a small amount of water to keep the arteries moist.
5. Check the tubing for air bubbles. If bubbles exist, turn on the pump and allow the 0.9% saline to flow until all bubbles are removed.

### 5. Application of the Dressing

1. Open the haemostatic dressing pouch and remove haemostatic dressing
2. Place the haemostatic dressing, mesh backing side UP, over the hole in the artery
3. Slowly wet the haemostatic dressing with an amount of saline appropriate for the article being tested

### Note:

A standard (13-15 mg/cm² of fibrinogen) 2.4×2.4 cm haemostatic dressing should be wet with 800 μl of saline or other blood substitute. The amount of saline used can be adjusted depending on the requirements of the particular experiment being performed; however, any changes should be noted on the data collection forms.

### Note:

Wet the haemostatic dressing drop wise with 0.9% saline warmed to 29-33° C. or other blood substitute, taking care to keep the saline from running off the edges. Any obvious differences in wetting characteristics from the positive control should be noted on data collection forms.

4. Place the shield gently onto the haemostatic dressing, taking care that it lies flat between the O-rings. Press lightly to secure in place
5. Wrap the artery and haemostatic dressing with plastic wrap
6. Wrap with blood pressure cuff, taking care that the bladder is adjacent to the haemostatic dressing.
7. Pump up the bladder to 100-120 mmHg, and monitor the pressure and pump again if it falls below 100 mmHg. Maintain pressure for 5 minutes.

### Note:

Time and pressure can be altered according to the requirements of the experiment; changes from the standard conditions should be noted on the data collection forms.

8. After polymerization, carefully unwrap the artery and note the condition of the haemostatic dressing. Any variation from the positive control should be noted on the data collection form.

Exclusion Criterion:

The mesh backing must remain over the hole in the artery. If it has shifted during the polymerization and does not completely cover the hole the haemostatic dressing must be excluded.

Testing Procedure

1. Diagram of Testing Equipment Set-Up

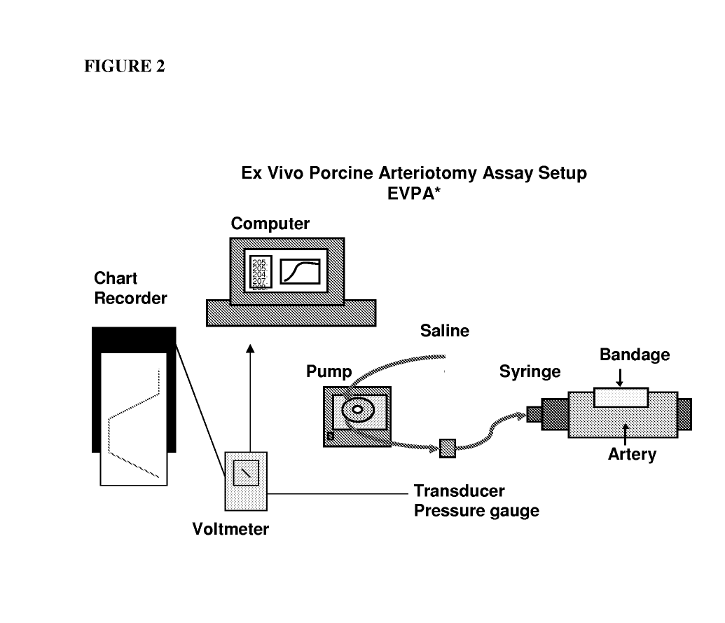

The set-up of the testing equipment is shown in FIG. 2. Some additional, unshown components may be utilized to read out (pressure gauge) or control the pressure within the system

2. Equipment and Artery Assembly

Fill the artery and syringe with red 0.9% saline warmed to 37° C., taking care to minimize the amount of air bubbles within the syringe & artery. Filling the artery with the opening uppermost can assist with this. Attach the artery and syringe to the testing apparatus, making sure that there are as few air bubbles in the tubing as possible. The peristaltic pump should be calibrated so that it delivers approximately 3 ml/min. If available, the PLC should be operated according to a pre-determined range of pressures and hold times as appropriate for the article being tested. If under manual control, the pressure/time profile to be followed is attained by manually turning the pump on and off while referencing the system pressure as read out by one or more pressure-reading components of the system. Following the conclusion of testing, the haemostatic dressing is subjectively assessed with regard to adhesion to the artery and formation of a plug in the artery hole. Any variations from the positive control should be noted on the data collection form.

### Success Criteria

Haemostatic dressings that are able to withstand pressures for 3 minutes are considered to have passed the assay. When a haemostatic dressing has successfully passed the assay the data collection should be stopped immediately so that the natural decrease in pressure that occurs in the artery once the test is ended isn't included on the graphs. Should the operator fail to stop data collection, these points can be deleted from the data file to avoid confusing the natural pressure decay that occurs post-test with an actual dressing failure. The entire testing period from application of the haemostatic dressing to completion must fall within pre-established criteria. The maximum pressure reached should be recorded on the data collection form.

### Note:

Typical challenge is 250 mmHg for three minutes in one step, but that may be altered based on the article being tested. Changes from the standard procedure should be noted on the data collection forms.

Failure criteria

Haemostatic dressings that start leaking saline at any point during testing are considered to have failed the assay.

### Note:

Build failures that are caused by artery swelling can be ignored and the test continued or re-started (as long as the total testing time doesn't fall beyond the established limit).

When leakage does occur, the pressure should be allowed to fall ˜20 mmHg before data collection is stopped so that the failure is easily observed on the graphs. The pressures at which leakage occurred should be recorded on the data collection form. Should the data collection stop in the middle of the experiment due to equipment failure the data can be collected by hand at 5 second intervals until the end of the test or haemostatic dressing failure, whichever happens first. The data points should be recorded on the back of the data collection form, clearly labeled, and entered by hand into the data tables.

### Exclusion Criteria

If the total testing period exceeds the maximum allowed for that procedure, regardless of cause, results must be excluded. If there are leaks from collaterals that can't be fixed either by patching or finger pressure the results must be excluded. If the test fails because of leaks at the O-rings, the results must be excluded. If the mesh backing does not completely cover the hole in the artery, the results must be excluded.

Adherence Performance Testing

1. Equipment and Supplies

Hemostat(s), Porcine artery and haemostatic dressing (usually after completion of the EVPA Assay although it does not need to be performed to do the Adherence Assay)

### I. Preparation of the Artery+Dressing

After application of the dressing without completion of the EVPA Assay, the dressing is ready for the Adherence Assay and Weight Limit Test (if applicable). After application of the dressing and subsequent EVPA Analysis, the artery and syringe system is then disconnected slowly from the pump so that solution does not spray everywhere. The warmed, red saline solution from the EVPA Assay remains in the syringe until the Adherence Assay and Weight Limit Test (if applicable) is completed.

Performance of the Adherence Assay

1. After preparation of the artery and dressing (with or without EVPA analysis), gently lift the corner of the mesh and attach a hemostat of known mass to the corner.

### Note:

If the FD developed a channel leak during the performance of the EVPA Assay, test the adherence on the opposite of the haemostatic dressing to obtain a more accurate assessment of the overall adherence.

2. Gently let go of the hemostat, taking care not to allow the hemostat to drop or twist. Turn the syringe so that the hemostat is near the top and allow the hemostat to peel back the dressing as far as the dressing will permit. This usually occurs within 10 seconds. After the hemostat has stopped peeling back the dressing, rate the adherence of the bandage according to the following scale:

| Dressing Performance Score | Amount of Adherence |
|---|---|
| 4 | 90+% |
| 3 | 75-90% |
| 2 | 50-75% |
| 1 | ~50% |
| 0.5 | Only the plug holds the hemostat |
| 0 | No adherence |

### Exclusion Criteria

The mesh backing must remain over the hole in the artery. If it has shifted during the polymerization and does not completely cover the hole the haemostatic dressing must be excluded.

### Success Criteria

Dressings that are given an adherence score of 3 are considered to have passed the assay.

### Failure Criteria

If a dressing does not adhere to the artery after application and/or prior to performing the EVPA assay, it is given a score of 0 and fails the adherence test. If a dressing receives a score ≦2, the dressing is considered to have failed the Adherence Assay.

### Weight Held Performance Assay

After the initial scoring of the “Adherence Test”, weights may then be added to the hemostat in an incremental manner until the mesh backing is pulled entirely off of the artery. The maximum weight that the dressing holds is then recorded as a measure of the amount of weight the dressing could hold attached to the artery.

### Moisture Assay

Moisture determinations were carried out using a Brinkman Metrohm Moisture Analyzer System. The system contains the following individual components, 774 Oven Sample Processor, 774SC Controller, 836 Titrando, 5 ml and 50 ml 800 Dosino Units and a 801 Stirrer. The system was connected to a computer using the Brinkman Tiamo software for data collection, analysis and storage. The moisture system is set-up and run according to the manufactures recommendations and specifications to measure the moisture content of lyophilized samples using the Karl Fischer method.

All components were turned on and allowed to reach operating temperature prior to use. Lactose and water were run as standards and to calibrate the instrument. Once the machine was successfully calibrated, samples were prepared as follows. Dressing pieces weighing at least 30 mg were placed into vials and capped. The vials were placed in the 774 Oven Sample Processor in numerical order, and one empty capped vial is placed in the conditioning space. The machine was then run to determine the moisture content (residual moisture) in the controls and samples.

### SDS-PAGE Gel Electrophoresis

Each dressing is cut into ¼'s, approximately 50 mg per section, and a section is then placed into a 15 mL conical tube. For the production control (i.e. Time 0), 1.0 mL of Okuda Dissolving Solution (10 M Urea, 0.1% Sodium Dodecyl Sulfate, 0.1% β-Mercaptoethanol) is added. For the remaining 3 pieces, 80 μL of 0.9% Saline is added to wet the dressing. The pieces are then incubated at 37° C. for 2, 5, and 10 minutes or such time as desired. To stop the reaction at the desired time, 1.0 mL of the Okuda Dissolving solution is added. The samples are then incubated at room temperature overnight, and then incubated at 70° C. for 30 minutes.

To prepare the samples for loading onto the gel, the samples which were previously dissolved in the Okuda Dissolving Solution were added to Sample buffer so that a 20 μL aliquot contains 10 μg. One μL of 0.1 M Dithiothreitol was then added to each sample. Twenty μL of each diluted sample is then loaded onto an 8% Tris-Glycine gel (Invitrogen), 1.0 mm thick, 10 wells. The gels were then run at 140V until the dye front reached the end of the gel. They were then removed and placed into Coomassie Blue Stain (50% v/v Methanol, 0.25% w/v Coomassie Brilliant Blue, 10% w/v Acetic Acid in ddH2O) on a shaking platform for a minimum of 1 hour. The gel is then transferred to the Destain Solution (25% Methanol, 10% Acetic Acid, 65% ddH2O) on a shaking platform until the background is nearly colorless.

After destaining, the gels were scanned, and the γ-γ dimer bands and the Aα, and Bβ bands analyzed by Scion densitometry software in order to determine the amount of conversion that occurred.

1. A solid dressing comprising a solid haemostatic layer wherein said solid haemostatic layer comprises dried fibrinogen component and thrombin, said dried fibrinogen and thrombin being dried from a single aqueous mixture of fibrinogen and thrombin.

2. A solid dressing comprising a haemostatic layer comprising substantially unreacted fibrinogen component and thrombin, wherein said fibrinogen component and said thrombin are dried from a single aqueous solution forming said solid haemostatic layer; and wherein said fibrinogen component and thrombin within the solid are substantially unreacted until said solid dressing comes into contact with an aqueous fluid.

3. The solid dressing of claim 1 comprising at least two haemostatic layers.

4. The solid dressing of claim 1 or 2, further comprising at least one support layer.

5. The solid dressing of claim 4, wherein said support layer comprises a backing material.

6. The solid dressing of claim 4, wherein said support layer comprises an internal support material.

7. The solid dressing of claim 4, wherein said support layer comprises a resorbable material.

8. The solid dressing of claim 4, wherein said support layer comprises a non-resorbable material.

9. (canceled)

10. The solid dressing of claim 4, further comprising at least one physiologically acceptable adhesive between said haemostatic layer and said backing layer.

11-13. (canceled)

14. The solid dressing of claim 1, wherein said haemostatic layer also contains a fibrin cross-linker and/or a source of calcium ions.

15. The solid dressing of claim 1 or 2, wherein said haemostatic layer also contains one or more of the following: at least one filler; at least one solubilizing agent; at least one foaming agent; and at least one release agent.

16. The solid dressing of claim 3, wherein said haemostatic layer further contains at least one binding agent in an amount effective to improve the adherence of said haemostatic layer to said support layer.

17-29. (canceled)

30. A solid dressing comprising a haemostatic layer comprising a substantially unreacted mixture of fibrinogen component and fibrinogen activator molecules; wherein said fibrinogen component and fibrinogen activator molecules are present in a single frozen aqueous medium forming said solid haemostatic layer.
